(19) United States
(12) Patent Application Publication
(10) Pub. No.: US 2026/0077027 A1
(43) Pub. Date: Mar. 19, 2026

(54) PEPTIDE AND NUCLEIC ACID METHODS TO MODULATE DELIVERY OF NUCLEIC ACID STRUCTURES, POLYPEPTIDES, AND THEIR CARGOES

(71) Applicant: Ohio State Innovation Foundation, Columbus, OH (US)

(72) Inventors: Niksa ROKI, Columbus, OH (US); Carlos CASTRO, Columbus, OH (US); Christopher LUCAS, Columbus, OH (US); Meixiao LONG, Columbus, OH (US); John BYRD, Columbus, OH (US)

(21) Appl. No.: 19/105,977

(22) PCT Filed: Aug. 24, 2023
(86) PCT No.: PCT/US2023/072787

§ 371 Date: Feb. 24, 2025

Related U.S. Application Data
(60) Provisional application No. 63/373,492, filed on Aug. 25, 2022.

(51) Int. Cl. A61K 39/00; A61P 37/04; C07K 14/435

(52) U.S. Cl. CPC A61K 39/0011; A61P 37/04; C07K 14/435; A61K 2039/55561; A61K 2039/6025; A61K 2039/627; A61K 2039/70; C07K 2319/00

(57) ABSTRACT

Disclosed herein are methods and compositions for enhancing delivery and function of vaccine components, immunotherapy Agents, and improved delivery of nucleic acid nanostructures, nucleic acids, peptides, polypeptides, and other types of cargoes. These methods and compositions utilize design components suitable for rapid and cost-effective manufacturing, and are designed to exclusively use the process of self-assembly to form nanotherapeutics requiring no purification in many instances.

### CROSS-REFERENCE TO RELATED APPLICATIONS

This application claims benefit of U.S. Provisional Application No. 63/373,492, filed Aug. 25, 2022, which is hereby incorporated herein by reference in its entirety.

### STATEMENT REGARDING FEDERALLY SPONSORED RESEARCH OR DEVELOPMENT

This invention was made with Government Support under Grant Nos. CA197734 and HL141941 awarded by the National Institutes of Health. The Government has certain rights in the invention.

### SEQUENCE LISTING

This application contains a sequence listing filed in ST.26 format entitled “321502-2080 Sequence Listing” created on Aug. 23, 2023, having 5,376 bytes. The content of the sequence listing is incorporated herein in its entirety.

### BACKGROUND

Cancer vaccines may be useful immunotherapy approach. Cancer vaccines contain tumor antigens designed to stimulate APCs and cytotoxic T cells that can help eradicate tumors, while providing immunological memory to prevent relapse (Houghton, A N & Guevara-Patiño, J A. J Clin Invest 2004 114:468-471). Unlike prophylactic vaccines that are generally administered against specific and highly immunogenic non-self antigens, and to individuals with a healthy functioning immune system, tumor vaccines require overcoming several challenges associated with the complex and less understood interplay between the immune system and tumor microenvironment (Houghton, A N & Guevara-Patiño, J A. J Clin Invest 2004 114:468-471; Fleming, V, et al. Front. Immunol. 2018 9). These challenges include safety concerns, poor immunogenicity and specificity of tumor antigens, immune suppression by the tumor, and tumor antigen heterogeneity, which ultimately lead to suboptimal immune responses against cancer (Lou, J, et al. Advanced Therapeutics 2019 2:1800128). To overcome these challenges, novel delivery technologies that can integrate and precisely synchronize multiple immune regulating functions and a broad spectrum of antigens for robust tumor-specific immunogenicity are required. Importantly, as new neoantigens are being discovered, and patient sample are being screened for patient-specific tumor antigens, new technologies are needed that support timely integration of patient specific antigens in cancer vaccine design. Currently, novel technologies exist and more are in development to be able to rapidly synthesise peptides. However, peptides are in general suboptimal antigens, requiring their delivery in form of nanoparticles and in combination with appropriate adjuvants, to improve their delivery and vaccine function. In addition, current vaccines fail to induce proper immune responses against variety of diseases still impacting the world, such as AIDS, malaria, etc.

Nanoparticle (NP) geometry, surface organization, density, and types of molecules play one of the key roles in determining interactions of nanomedicines with biological environment, therefore affecting therapeutic index of nanomedicines, including nanoparticle-based cancer vaccines, as well as general vaccines. For example, NP immunorecognition, adjuvancy and antigenicity, biodistribution, clearance, pharmacokinetics and pharmacodynamics, cell binding, internalization, and intracellular fate are some of the biological processes that are affected by NP properties. This has been demonstrated for vaccine delivery, particularly regarding geometry dictating organization of antigens on nanoparticle surface. In this regard, nanoparticles have the ability to mimic pathogens, therefore enhancing adjuvancy and antigenicity of the molecules used in vaccines.

DNA nanotechnology provides exquisite precision and control over nanoparticle design, manufacturing, reproducibility, and scalable and cost-effective manufacturing. For example, DNA origami, a method to fold DNA strands into 2- and 3-dimensional geometries, provides unprecedented control over NP geometry, including control over surface molecule organization. Therapeutics, targeting ligands, protecting agents, etc., can all be organized by precise attachment to precisely placed nucleic acid-based attachment arms via base complementarity. However, although DNA-made delivery systems have intrinsic ability for high capacity and loading efficiency of intercalating drugs, DNA nanoparticles either have limited intrinsic ability to load therapeutics (e.g., peptides, proteins, nucleic acids, small molecules, etc.) and achieve high payload density per nanoparticle via complementary base pairing or conjugation due to the limited single-stranded oligonucleotide-based attachment sites, such as in the case of DNA origami where additional attachment sites compromise the integrity and stability of the structure, or have limited ability to precisely control the 3D shape of single-stranded oligonucleotide-based attachment sites presentation, such as in the case of DNA branched dendrimers that have very limited design space regarding their geometry. Another limitation of DNA branches (i.e., DNA dendrimers), is that as these nanostructures are made larger and larger, their density of branching reduces due to radial growth outward, while at the same time having a limitation of having a requirement for minimal number of bases needed for stable double stranded complementary binding between each branching subunit.

Furthermore, while attaching nucleic acid-based therapeutics, such as CpG adjuvant, to nucleic acid-based nanoparticles is very efficient, affordable, and avoids complexity associated with other NP systems, other therapeutics such as peptides and proteins require modifications with oligonucleotides, and often extensive and costly purifications, before they can be attached to nucleic acid-based NPs. Importantly, this can delay manufacturing of patient-specific tumor peptide antigens after screening of tumor patient samples. On the other hand, electrostatic-based attachments, or insertions in lipid nanoparticles, while often cost-effective, scalable, and sufficient for attaching protective molecules without the intended therapeutic activity, such as polyethylene glycol (PEG), these methods, particularly electrostatic-based attachment, have been unreliable to deliver therapeutics either due to their instability, or unfavorable NP charge. Suboptimal NP charge is the main safety concern due to issues with aggregation and non-specific tissue and cell binding. Hence, such formulations are particularly challenging to develop for I.V. route administration, where decoupling of therapeutic components may result in detrimental consequences. For example, delivering peptide antigens to immune cells decoupled from the adjuvant vaccine components partially defeats the purpose of NP-based delivery and can result in antigen tolerization depending on the nature of antigen instead of successful immunization against that particular antigen.

### SUMMARY

Disclosed herein are methods and compositions for enhancing delivery and function of vaccine components, immunotherapy agents, and improved delivery of nucleic acid nanostructures, nucleic acids, peptides, polypeptides, and other types of cargoes. These methods and compositions utilize design components suitable for rapid and cost-effective manufacturing, and are designed to exclusively use the process of self-assembly to form nanotherapeutics requiring no purification in many instances. As will be demonstrated in this document, these compositions are uniquely and in a novel way compatible with alginate encapsulation strategy. Disclosed herein is a vaccine device based on LL37 chimeric peptide complexation with DNA branched origami delivery platform (OriBranch), comprising a core DNA nanostructure (e.g., DNA origami) formed from a plurality of scaffold strands and a plurality of staple strands assembled into a geometry, wherein the DNA nanostructure comprises one or more first single stranded DNA oligonucleotide attachment arms configured to bind to a first complementary DNA oligonucleotide strands, further comprising a branched oligonucleotide dendrimer comprising a plurality of second single stranded DNA oligonucleotide attachment arms and at least one first complementary DNA oligonucleotide strand, wherein the second single stranded DNA oligonucleotide attachment arms are configured to bind to second complementary DNA oligonucleotide strands, wherein an LL37-based chimeric peptide or polypeptide antigen is attached to the DNA of the DNA branched origami nanostructure by specific binding of LL37 portion to DNA, and which can further be encapsulation within alginate. One of the many unique feature of such organized DNA branches, is that the origin for each DNA branch attachment is precisely determined on DNA origami, and the angle of the growth of these DNA branches can be precisely adjusted, not only by the frequency of branching, but also by the geometry of DNA nanoparticle, where the surface of DNA origami nanoparticle can be even designed concave, to provide the greatest control over the density of DNA branching units on DNA NP and therefore free attachment arms in 3D space. Using this particular method, theoretical maximum of attachment arms per assigned 3D space can be achieved, while surpassing the limitations of the minimum number of base pairs in double stranded regions necessary for double strand stability.

Disclosed herein are DNA branched structures of precise sequence and precise length, and precisely organized by DNA origami nanoparticles, loaded with CpG adjuvants and proteins via oligonucleotide complementary binding and antigenic polypeptides via LL37-nucleic acid binding chimeric peptides containing antigen peptide sequences, and encapsulated within alginate capsules, as well as their methods for enhanced nucleic acid structures, peptides, and their cargo delivery. The DNA branched origami nanostructures have the ability to enhance cargo delivery (e.g., CpG adjuvant and peptide antigens) as compared to cargo molecules alone or cargo molecules carried by common DNA nanostructures. The DNA branched origami nanostructures have the ability for enhanced payload attachment either via oligonucleotide-based complementary attachment to free arms located on many branches on the surface of DNA origami, or via electrostatic peptide attachment. The DNA branched structures alone or self-assembled on DNA origami have the ability to reduce nanoparticle aggregation induced by electrostatic interactions of peptides, by unknown mechanism as compared to regular DNA origami structures. LL37-based attachment of polypeptides to DNA structures has the ability to reduce non-specific interactions with biological environment as compared to commonly employed lysine—or arginine—(e.g., K10) based molecule (e.g., peptide) attachment. LL37-based attachment of polypeptides to DNA structures is compatible with phosphorothioated oligonucleotide attachment to the free attachment arms of DNA branched origami nanostructures and protects and stabilizes this attachment under conditions of degradation as compared to K10-based attachment. LL37-based attachment of polypeptides to DNA structures has the ability to protect structures and their cargo from degradation, and further encapsulation within alginate capsules extends this protection even further. The DNA branched origami nanostructures have the ability to enhance antigen presentation and adjuvant stimulation of CpG adjuvant and peptide antigen payload as compared to free CpG adjuvant and peptide antigen. The DNA branches or DNA branched origami nanostructures have the ability to carry CpG adjuvant molecules by double stranded attachment along the full length of CpG sequence, while preserving CpG function. LL37-chymeric polypeptides have the ability to be stably retained within alginate capsules for extend period of time and be protected from degradation. CpG adjuvant-loaded DNA branched or DNA branched origami structures complexed with LL37-peptide antigen chimeras have the ability to enhance vaccination focusing on inducing T cell killing function as compared to CpG adjuvant mixed with peptide antigens. LL37-peptide antigen chimeras complexed with CpG-loaded DNA branched nanostructures has the ability to induce cytotoxic T cell-based vaccination via I.V. administration route despite electrostatic-based peptide attachment and nanoparticle charge neutralization. LL37-peptide antigen complexed with CpG adjuvant has the ability to slow down the tumor growth and prolong survival. CpG-loaded DNA branched structures complexed with LL37-peptide antigen and encapsulated in alginate capsules have the ability to rapidly shrink tumor size and prolong survival. These formulations and methods are useful for enhancing the delivery and functions of various molecules such as adjuvants, antigens, LL37 molecules, etc. for purposes of immunotherapy, vaccination, autoimmune diseases, allergies, wound healing, etc.

Results presented in this disclosure demonstrate that, DNA branched origami enhances CpG nucleic acid adjuvant and peptide antigen delivery, enhances immune stimulation and antigen presentation, and enhances T cell killing function relative to free CpG adjuvant and free peptide antigen administration. This finding cannot be solely explained by the nanoparticulate or branched nature of DNA branched origami, as this level of enhancement is not seen with either DNA branch or DNA origami counterparts, although both counterparts enhance these same properties relative to free CpG and peptide antigen.

Furthermore, unexpectedly and unpredictably, DNA branched origami prevents, reduces, or delays aggregation commonly induced by electrostatic-based binding with peptides containing positive amine charges that neutralize negative DNA charges commonly leading to aggregation. The mechanism for this unique behavior is not known, but the results demonstrate that it is due to the DNA branched surface on DNA origami, as DNA branches alone were also discovered to have this property.

Unexpectedly, it was also discovered that DNA branches complexed with LL37-based chimeric peptide antigens are suitable for peptide-based vaccination via I.V. route, despite the negative DNA charge neutralization by LL37 positive charge and expected decoupling of DNA and LL37 in the bloodstream. Commonly, DNA NPs, and particularly DNA NPs with neutralized negative charges, are prone to aggregation, sequestration by the liver, and premature secretion via kidneys, as is known and demonstrated challenge for DNA-based NPs, and NPs in general. However, this I.V. route compatibility seems to be a unique property of LL37-based attachment to DNA branches or DNA branched origami since it was discovered that K10-based attachment causes drastically different properties. For example, K10 greatly enhances antigen presentation in combination with CpG-loaded DNA branched origami NPs in cell culture relative to nonmodified free antigenic peptides or LL37-based attachment system, which is most likely due to the demonstrated K10 system severe nonspecific binding to not only cells (and hence improved peptide antigen delivery), but also extracellular matrix mimic, gelatin-coated coverslips in the case of cell culture. On the other hand, it is demonstrated that LL37 peptide avoids nonspecific binding in cell culture, as well as efficiently mediates delivery of DNA branches to lymph nodes in vivo. Biodistribution of CpG-loaded DNA branches complexed with LL37 chimeric antigen peptides was more favorable as compared to free CpG.

Another unexpected property of LL37-based peptide attachment was that this specific sequence supported and maintained phosphorothioated CpG attachment to DNA branches or DNA branched origami NPs under degradative conditions of DNase I or fetal bovine serum as compared to K10-based peptide attachment that failed to maintain this oligonucleotide based attachment.

Lastly, very surprisingly, and very unexpected result, was the tumor size reduction within 2 days post intratumoral injection of the two subsequent half doses of the composition consisting of LL37 chimeric gp100 peptide antigen complexed with CpG-loaded DNA branches and encapsulated in alginate. Particularly unpredictable was the rapid reduction in tumor size in B16-F10 mouse melanoma model, instead of prolonged release and stimulation by encapsulation in alginate. This unexpected result cannot be explained solely by the presence of alginate and other components of the composition, as all the components were commercially obtained or custom synthesized with no detectable endotoxin levels, or at the lowest endotoxin level available in the case of alginate, and have not been previously reported to cause such a drastic effect when administered separately. On the other hand, another unexpected, and conflicting discovery in regards to this newly discovered phenomenon of alginate-encapsulated, LL37gp100-complexed and CpG-loaded DNA branches is not only that this formulation is stable within alginate for extended period of time as demonstrated by release and degradation studies, but the LL37 peptides are stable in alginate on their own, which is not expected for peptides of that size and can certainly not explain rapid therapeutic effect of aforementioned composition involving alginate, LL37 chimeric antigen peptides, CpG, and DNA branched nanostructures.

In some embodiments, the peptide can further be attached to or be a part of the sequence of other peptide, or protein, or other molecules or functions desired to be attached or performed such as nuclear localization peptide sequences, cell membrane penetrating sequences, etc.

In some embodiments, the peptide antigen comprises LL37 chimeric peptide sequence extended with another peptide, polypeptide, or protein of interest.

In some embodiments, the peptide antigen comprises cell penetrating peptides and/or antimicrobial peptides at the N- and/or C-terminus for tuning the peptide electrostatic attachment to DNA nanostructures, increased cell uptake, cytosolic and/or nuclear delivery containing for example LL37 sequence: LLGDFFRKSKEKIGKEFKRIVQRIKDFLRNLVPRTESSIINFEKL, where SIINFEKL portion of the sequence is a model antigen peptide that can be replaced with any antigen peptide sequence.

In some embodiments, the peptide antigen comprises at least 5, 6, 7, 8, 9, or 10 contiguous positively charged amino acids.

In some embodiments, the at least 5, 6, 7, 8, 9, or 10 continuous positively charged amino acids are at the N-terminus of the peptide antigen. In some embodiments, the at least 5, 6, 7, 8, 9, or 10 continuous positively charged amino acids are at the C-terminus of the peptide antigen.

In some embodiments, the positively charged amino acids are lysine or arginine amino acids. In some embodiments, the peptide antigen comprises at least 10 contiguous lysine amino acids. In some embodiments, the peptide antigen comprises at least 10 contiguous arginine amino acids.

In some embodiments, at least 1,000 peptide antigens are attached to the DNA nanostructure. In some embodiments, the vaccine device has at least 30, 35, 40, 45, 50, 60, 70, 80, 90, 100 peptides per nm². In some embodiments, the vaccine device has at least 50, 60, 70, 80, 90, 100 nM peptide per NP. In some embodiments, the DNA branched origami NP has peptide attached at the concentration of at least 2.5 mM within the space or volume of single NP.

In some embodiments, each of the plurality of scaffold strands are 5,000 to 10,000 nucleotides in length. In some embodiments, each of the plurality of scaffold strands are derived from a virus. In some embodiments, each of the plurality of scaffold strands are derived from bacteriophage M13. In some embodiments, each of the plurality of scaffold strands are synthetic and/or non-immunogenic. In some embodiments, each of the plurality of scaffold strands are mRNA.

In some embodiments, the peptide antigen comprises a viral antigen. In some embodiments, the peptide antigen comprises a tumor specific antigen and/or tumor associated antigen.

In some embodiments, the core DNA nanostructure comprises one or more first single stranded DNA oligonucleotide attachment arms configured to bind to a first complementary DNA oligonucleotide strands.

In some embodiments, the core DNA nanostructure is integrated with at least one non-nucleic acid material.

In some embodiments, the vaccine further contains a plurality of nucleic acid adjuvant molecules conjugated to first complementary DNA oligonucleotide strands or second complementary DNA oligonucleotide strands.

In some embodiments, the plurality of nucleic acid adjuvant molecules are CpG molecules.

In some embodiments, the DNA nanostructure is a DNA origami nanostructure. In some embodiments, the DNA origami nanostructure is assembled into a rod shape. In some embodiments, the DNA origami nanostructure is assembled into a cube shape. In some embodiments, the DNA origami nanostructure is assembled into a spherical shape. In some embodiments, the DNA origami nanostructure is assembled into a rigid shape. In some embodiments, the DNA origami nanostructure is assembled into a flexible shape. In some embodiments, the DNA origami nanostructure comprises a cavity, wherein the one or more first single stranded DNA oligonucleotide attachment arms are positioned inside the cavity and attached to DNA branches via oligonucleotide complementarity. In some embodiments, the DNA branches are integrated throughout the whole volume of DNA origami nanostructure or other non-branching DNA nanoparticle.

In some embodiments, vaccine device further comprises one or more targeting ligands conjugated to the first complementary DNA oligonucleotide strands or second complementary DNA oligonucleotide strands containing DNA branches.

In some embodiments, the one or more targeting ligands are DNA aptamers, peptide aptamers, peptides, or antibodies.

In some embodiments, each branched oligonucleotide dendrimer integrated with core nucleic acid nanoparticle comprises at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 second single stranded DNA oligonucleotide attachment arms.

In some embodiments, the DNA-based nanostructure is encapsulated in an alginate capsule.

In some embodiments, the DNA nanostructures are complexed with LL37 peptide chimeras and then encapsulated in an alginate capsules.

Also disclosed herein is a method for vaccinating a subject, comprising administering to the subject a vaccine device disclosed herein.

In some embodiments, the peptide antigen is attached to the nucleic acids of the preformed nanostructure by means of LL37 peptide (LLGDFFRKSKEKIGKEFKRIVQRIKDFLRNLVPRTES) attachment.

Also disclosed herein is a nucleic acid nanostructure, comprising a nucleic acid-made branches incorporated with a DNA origami nanoparticle, where a DNA origami technique is used to direct and organize the origin of nucleic acid-made branches, while nucleic acid-based branches control the topography and density of the structure, including the attachment sites for various molecules.

In some embodiments, the nucleic acid nanostructure is further complexed with peptides based on electrostatic attachment or attachment with DNA binding molecules such as LL37 peptide, anthracyclines, etc.

In some embodiments, the DNA origami nanoparticle is substituted with any non-branching nucleic acid-based nanoparticle containing the attachment arms to bind nucleic acid branches via nucleic acid complementary base pairing, where nucleic acid branches are in their entirety made out of nucleic acids.

In some embodiments, the nucleic acid branches on nucleic acid nanoparticles modulate and/or enhance the solubility and stability of the DNA origami structure, such as for example after electrostatic complexation with peptides.

In some embodiments, the nucleic acid branches on nucleic acid nanoparticles increase the nucleic acid surface area of nucleic acid nanoparticles for enhanced functions such as increased loading of electrostatically bound peptides onto DNA origami.

In some embodiments, the nucleic acid branches on nucleic acid nanoparticles create collapsable cavities to provide protection of molecules, including electrostatically bound peptides, and intraparticle aggregation of electrostatic peptides to reduce interparticle aggregation.

In some embodiments, the nucleic acid branches on nucleic acid nanoparticles provide a novel means to control topography, density, and spacing of functions and molecules of interest to be incorporated or attached on nucleic acid nanoparticles, such as aptamers, antibodies, fatty acids, cholesterol, phospholipids, adjuvants, antigens, etc. Also disclosed herein is a nucleic acid dendrimer nanostructure that is made by self-assembly of unique predefined sequences at predefined molar ratios in one step (i.e., one pot synthesis) in the presence of only 1× phosphate buffered saline and require no purification procedures after the self-assembly process.

Also disclosed herein is a nucleic acid dendrimer nanostructure electrostatically complexed with peptides or portions of peptide containing amino acids with amine groups, guanidine groups, or positive charges.

In some embodiments, the peptides comprise an LL37 peptide.

Also disclosed herein is a nucleic acid dendrimer nanostructure electrostatically complexed with peptides or portions of peptide containing cell penetrating peptides.

Also disclosed herein is a method of protecting nucleic acid dendrimer nanostructure via electrostatic complexation with peptides.

Also disclosed herein is a method of protecting nucleic acid nanostructures via electrostatic complexation with LL37 peptides or peptides containing LL37 sequence.

Also disclosed herein is a method of enhancing cytosolic delivery via electrostatic complexation of nucleic acid nanostructures with LL37 peptides or peptides containing LL37 sequence.

Also disclosed herein is a method of enhancing cytosolic delivery of nucleic acid origami nanostructures (e.g., DNA origami nanoparticles) via decoration of nucleic acid origami nanostructures with nucleic acid branched structures.

Also disclosed herein is a method of enhancing cytosolic delivery of nucleic acid origami branched nanostructures via electrostatic complexation of nucleic acid nanostructures with LL37 peptides or peptides containing LL37 sequence.

Also disclosed herein is a method of increasing electrostatic-based peptide attachment to nucleic acid origami nanostructures via incorporation of nucleic acid branched dendrimer structures.

Also disclosed herein is a method of increasing molecule function and/or attachment to nucleic acid origami nanostructures via incorporation of nucleic acid branched dendrimer structures.

Also disclosed herein is a method of controlling molecule spacing, including real-time adaptable/flexible spacing to match exact distance needed for receptor dimerization or multivalent binding via incorporation of nucleic acid branched dendrimer structures onto nucleic acid origami nanostructures, where the two or more adjacent single stranded arms have the flexibility to bind to two or more distinct positions on dimer or other receptors with the distance range of zero to fifteen nanometers between the binding spots.

Also disclosed herein is a method of protecting nucleic acid origami structures and their cargo from premature degradation or premature therapeutic release (e.g., release of chemotherapy drugs and gene regulating molecules embedded or folded into the core of nucleic acid nanostructures) by subsequent attachment of a dense nucleic acid branched dendrimer network to the surface of nucleic acid structures after they have already been loaded with a cargo.

Also disclosed herein is a nucleic acid nanovaccine structure that can directly incorporate FDA approved oligonucleotide adjuvants by direct attachment to the nucleic acid structure, without the need for modification of oligonucleotide adjuvant, while preserving adjuvant function and potency.

Also disclosed herein is a vaccine formulation of peptide and nucleic acid adjuvants, wherein the peptide antigen comprises cell penetrating peptides and/or antimicrobial peptides at the N- and/or C-terminus for tuning the peptide electrostatic attachment to nucleic acid nanostructures, increased cell uptake, cytosolic and/or nuclear delivery containing for example LL37 sequence: LLGDFFRKSKEKIGKEFKRIVQRIKDFLRNLVPRTESSIINFEKL, where SIINFEKL portion of the sequence is a model antigen peptide that can be replaced with any peptide sequence.

Also disclosed herein is a vaccine formulation of peptide and nucleic acid adjuvants, wherein the peptide antigen comprises cell penetrating peptides and/or antimicrobial peptides at the N- and/or C-terminus for tuning the peptide electrostatic attachment to DNA nanostructures, increased cell uptake and cytosolic delivery, and further contains at least one peptide cleavage site (e.g., cathepsin cleavage site, furin cleavage site, etc.), and/or at least one immunoproteasome processing site for peptide processing containing the following example sequence: LLGDFFRKSKEKIGKEFKRIVQRIKDFLRNLVPRTESKLALRVRRALISLEQLESIINFEKL TEW), where SIINFEKL portion of the sequence is a model antigen peptide that can be replaced with any peptide sequence.

Also disclosed herein is a vaccine formulation of LLGDFFRKSKEKIGKEFKRIVQRIKDFLRNLVPRTESSIINFEKL peptide and nucleic acid adjuvants, where SIINFEKL portion of the sequence is a model antigen peptide that can be replaced with any antigen peptide sequence.

Also disclosed herein is a vaccine formulation of LLGDFFRKSKEKIGKEFKRIVQRIKDFLRNLVPRTESKLALRVRRALISLEQLESIINFEKL TEW peptide and nucleic acid adjuvants, where SIINFEKL portion of the sequence is a model antigen peptide that can be replaced with any peptide sequence, and where sequences such as KLALRVRRALISLEQLE and TEW, which have cell penetrating or peptide cleavage or proteasome directing functions can be replaced with different sequences performing similar functions.

In some embodiments, the peptide antigen comprises 1 to 5 contiguous positively charged amino acids. In some embodiments, the peptide antigen comprises 1 to 3 contiguous positively charged amino acids.

Also disclosed herein is a method wherein nucleic acid nanostructure is electrostatically attached with peptides, wherein at least two or more distinct and distanced locations by more than 20 bases apart on the nucleic acid nanostructure are electrostatically contacting and binding peptides simultaneously, such as the ability of the two opposite arms of branched nucleic acid structures to interact with peptide simultaneously.

Also disclosed herein is a method, wherein peptide is shielded from the external biological environment via its incorporation within the branched nucleic network.

Also disclosed herein is a nucleic acid structure, comprising a nucleic acid origami nanostructure formed from a plurality of scaffold strands and a plurality of staple strands assembled into a geometry, wherein the nucleic acid nanostructure comprises one or more first single stranded oligonucleotide attachment arms configured to bind to a first complementary oligonucleotide strands, further comprising a branched oligonucleotide dendrimer comprising a plurality of second single stranded oligonucleotide attachment arms and at least one first complementary oligonucleotide strand, wherein the second single stranded oligonucleotide attachment arms are configured to bind to second complementary oligonucleotide strands.

Also disclosed herein is a nucleic acid structure, comprising a nucleic acid origami nanostructure formed from a plurality of scaffold strands and a plurality of staple strands assembled into a geometry, wherein the nucleic acid nanostructure comprises one or more first single stranded oligonucleotide attachment arms configured to bind to a first complementary oligonucleotide strands, further comprising a branched oligonucleotide dendrimer comprising a plurality of second single stranded oligonucleotide attachment arms and at least one first complementary oligonucleotide strand, wherein the second single stranded oligonucleotide attachment arms are configured to bind to second complementary oligonucleotide strands, wherein a LLGDFFRKSKEKIGKEFKRIVQRIKDFLRNLVPRTESKLALRVRRALISLEQLESIINFEKL TEW is attached to the nucleic acids of the nanostructure, wherein SIINFEKL portion of the peptide can be replaced with any functional peptide.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic pertaining to an embodiment of a workflow towards optimizing a DNA branched origami vaccine (OriBranchVAC). An embodiment of the disclosed branched origami vaccine contains a DNA origami nanoparticle which represents a core nucleic acid nanoparticle construct containing free single-stranded oligonucleotide attachment arms for the attachment of nucleic acid branches (e.g., DNA branches). In this manner, nucleic acid branches are precisely positioned in 3D space around the core nucleic acid nanoparticle at a predetermined density, where nucleic acid branches have free arms for attaching CpG adjuvants, other nucleotide-based drugs, or any molecule that can be directly conjugated to these arms or conjugated to oligonucleotide arms complementary to the attachment arms of branches. Alternatively, some free arms on the core nanoparticle can be used to attach molecules to provide shielding from biological environment, such that these molecules are hidden in between the surface of the core nucleic acid nanoparticle and the coat of nucleic acid branches, where nucleic acid branches provide protection to the underlying molecule by steric hinderance and bio-physicochemical properties. Another alternative is that a molecule is incorporated within the core nucleic acid nanoparticle, such as for example DNA-binding molecule, where the layer of nucleic acid branches then serves as a sponge to sequester a DNA-binding molecule, therefore reducing leakage. Various DNA branched origami vaccine formulations were designed, using different number of branching units, and electrostatic-based polypeptide attachment methods. Peptide antigens can be assembled using chimeric polypeptides via electrostatic attachment, containing antigenic peptide region and a nucleic acid binding region, such as LL37 peptide sequence. These formulations were evaluated for cargo attachment and stability, size and geometry, cell binding, uptake, and trafficking, biodistribution, activation of antigen presenting cells and antigen delivery, T cell cytotoxic function and anti-tumor efficacy. These delivery methods may be useful in treatments against viruses, bacteria, and cancers, among many other applications.

FIG. 2 is a schematic pertaining to an embodiment of unique bio-physicochemical properties of branched origami vaccine formulations. The core nucleic acid nanoparticle can have a rigid or soft geometry (i.e., size and shape) of any kind, determining a specific shape for nucleic acid branch coat, while the length between each branching point, the number of units and branching points in each branch, and the surface density and chemical character of branches control the surface character. Furthermore, branched nucleic acids can be incorporated throughout the core nucleic acid nanoparticle, to modulate the compactness and flexibility of the core nanostructure. Branches provide a unique control of 3D topography on the core nucleic acid nanoparticle for various molecules such as targeting ligands and antigens, while providing flexibility and freedom of molecule motion as compared to non-branched nucleic acid structures; unique method to modulate ligand-targeting parameters via branched origami design to increase the number and flexibility of presentation of targeting ligands is shown. Nucleic acid branches can be attached to the core nucleic acid nanostructure on the surface or inside the cavities of the core nanoparticle, and maximize the loading capacity of the core nanostructure. Shown is incorporation of adjuvants into origami nanoparticle cavity to improve masking of adjuvants and reduce their release in circulation, which may improve vaccine safety. Next, shown is a concept design of branch assembly in the internal cavity to increase the attachment density inside the core nucleic acid nanoparticle. Lastly, alginate or other polymers can be used to encapsulate branched origami therapeutic for extended release and protection against degradation.

FIG. 3 is a schematic pertaining to an embodiment of chimeric LL37-polypeptide coated branch or branched origami subunit vaccine for combined peptide and protein antigen cargo loading. Presented are branch and branched origami delivery platforms utilizing dual antigen attachment technology consisting of oligonucleotide-based and electrostatic-based peptide and protein antigen attachment for strong induction of both, humoral and cellular immune responses. Branched design allows for high adjuvant, antigen, and ligand targeting attachment capacity simultaneously. The branched origami design has a precise and predetermined branch structure with terminal attachment arms whose number, density, and geometry are precisely defined, which can allow for targeting ligands to multiple target epitopes and to multiple cell types simultaneously (e.g., dendritic cells, macrophages, B cells, T cells, cancer cells, etc.) on a single branched origami nanostructure without compromising on the sufficient number of attached ligands or molecules; this is particularly useful for the ligands of relatively low affinity such as aptamers, or to direct one type of cell towards the other type of cell for various purposes. In addition, combination targeting to different target epitopes on the same cell type, as well as targeting multiple cell types is a known strategy to reduce off-site targeting; branched origami design may improve this strategy. Lastly, high coating of nucleic acid nanoparticles, particularly with proteins such as antibodies has been shown to protect DNA nanoparticle half-life in serum and in the body.

FIG. 4 is a schematic pertaining to an embodiment of direct CpG attachment to a nucleic acid nanostructure, where the free attachment arm sequence on the nucleic acid nanostructure is complementary to the full length of one of the commercially available or FDA approved nucleic acid sequences (e.g., CpG1826, etc), i.e., forming a double strand along the full length of CpG, which may give new properties to nucleic acid adjuvants.

FIG. 5 shows assembly of an embodiment of DNA branches in PBS and a direct CpG attachment to nucleic acid branch using agarose gel electrophoresis. Note that there was no shift in bands after CpG attachment due to the direct CpG attachment method, where the full 20 bases of CpG1826 are double stranded with DNA branch arms. Tight bands indicated precise and uniform product, while the lack of oligonucleotide bands under the band corresponding to DNA branch nanoparticles indicated complete annealing of oligonucleotides into a DNA branch, requiring no further purification steps.

FIGS. 6A to 6E show precise and efficient self-assembly of an embodiment of a high payload capacity DNA branched origami with various numbers of branched oligonucleotide arms for attachment of diverse cargoes of interest and with high cargo attachment efficiency. Tight bands indicated precise and uniform products. FIG. 6A shows agarose electrophoresis gels, that based on the band shifts at each step of synthesis, indicated successful manufacturing of DNA origami and CpG attachment on the left, while precise equimolar self-assembly of DNA branches on DNA origami and subsequent attachment of CpG is shown on the right. Again, note on the right that there was no shift in bands after CpG attachment to branched origami due to the direct CpG attachment method, where the full 20 bases of CpG1826 are double stranded with DNA branch arms. In comparison, it can be seen on the left that there was a shift when CpG is attached using a regular method where CpG is designed with a complementary oligonucleotide extension for attachment to DNA origami attachment arms. FIG. 6B shows a different number of starting attachment arms on DNA origami, subsequent precise equimolar self-assembly of DNA branches with different number of branching units and attachment arms, and finally, the direct attachment of CpG. DNA origami with and 64 attachment arms utilize common technique of designing attachment arms or overhangs in DNA origami field. Branched origami with 90, 150, and 320 branched attachment arms are examples of addition of DNA oligonucleotides designed using specific design rules to result in complete and efficient self-assembly of DNA branches on DNA origami. These rules include limiting self-binding sequence regions, limiting non target oligonucleotide binding, etc. FIG. 6C shows transmission electron microscopy (TEM, left) and atomic force microscopy (AFM, right) of DNA origami with 64 regular attachment arms showing uniform geometry, but regular attachment arms were too small to visualize as expected. FIG. 6D shows DNA branching layer encapsulating/layering DNA origami, where branches and/or branched attachment arms were clearly visible (TEM, left; AFM, right). The flexibility of branches and their collapse on origami surface was indicated by some of the branches being bent or not visible during drying process, often requiring AFM imaging in liquid conditions. FIG. 6E shows quantification of attachment of model molecule (fluorophore labeled CpG adjuvant) to regular or branched design attachment arms, demonstrating high attachment efficiency of over 94% and increasing payload capacity for increasing number of arm attachment sites. Fluorescence intensity, standard curve, and DNA optical density measurements were used to determine CpG concentration and DNA nanoparticle concentration for each sample after purification to determine the number of molecules attached per single NP.

FIGS. 7A to 7F show precise and uniform attachment, as well as function of proteins on DNA branched origami coupled via branched attachment arms. FIG. 7A shows agarose electrophoresis gels, that based on the band shifts as indicated by the red arrow, demonstrate efficient, complete, and precise coupling of proteins onto the DNA branched origami, where sub equimolar ratio-based attachment resulted in nearly maximum attachment capacity. Note that tight bands on the gel for protein conjugated DNA branched origami indicate uniform and monodisperse product. FIG. 7B shows TEM image of DNA branched origami fully coated with ovalbumin protein as a model antigen, demonstrating multivalent antigen presentation. FIG. 7C shows distinct shift in the gel band after electrophoresis for the antibody-oligonucleotide conjugate attachment to DNA branched origami loaded with CpG adjuvant; note that simply adding the oligonucleotide that was used for antibody conjugation did not result in the band shift. FIG. 7D shows TEM image of DNA branched origami on the left, and antibody decorated branched origami on the right. Since DNA branches on DNA origami are prone to collapsing during TEM sample preparation, making it difficult or impossible to visualize antibodies, AFM was performed (FIG. 7E) to demonstrate increase in thickness/height for the antibody-oligonucleotide loaded DNA branched origami as compared to oligonucleotide loaded DNA branched origami; approximately 1.5 nm increase in thickness was detected. Finally, FIG. 7E demonstrates enhanced adjuvancy, of CpG-loaded DNA branched origami specifically targeted to DEC205 receptors as compared to non-targeted counterpart in splenocyte co-culture focusing on professional antigen presenting cells (B cells, macrophages, and dendritic cells); dendritic cells, which are the main cell population for DEC205 marker, were specifically targeted as indicated by dendritic cell-specific enhancement of upregulation of CD80 and CD86, co-stimulatory molecules necessary for T cell activation.

FIG. 8 shows fluorescence signal from agarose gel electrophoresis, demonstrating CpG (shown in blue) colocalization with DNA origami or DNA branched origami bands and high efficiency payload capacity of peptide antigen (shown in green) loading via electrostatic attachment as compared to free peptide band that migrates in the opposite direction of DNA structures on the gel due to the K10 positive charges. DNA branched origami maximum peptide loading was confirmed for up to 2900 antigen molecules per single branched origami NP (right side of the gel) and was enhanced as compared to a regular DNA origami peptide loading of 1450 antigen molecules per single origami NP (left side of the gel).

FIGS. 9A and 9B show distinct differences in interaction of electrostatic peptides with DNA branched origami as compared to DNA origami. FIG. 9A show an image demonstrating appearance of DNA branched origami or DNA branches loaded with K10OVA or LL37OVA peptides as compared to regular DNA origami loaded with the same peptides. DNA branched origami NPs resisted, reduced, or delayed aggregation induced by electrostatic interactions between peptides and DNA NPs as compared to regular DNA origami, where aggregation was clearly visible upon mixing of electrostatic peptides and DNA origami. FIG. 9B shows a mechanism that may explain the unique interaction of branches or branch elements on DNA origami with electrostatic peptides. Depicted is an embodiment of nucleic acid branch properties in combination with nucleic acid-binding peptides giving rise to a novel means of peptide integration with nucleic acid branches; nucleic acid branches have negative charge, flexible waist and arms, and particularly important, multiple points of high flexibility, allowing for the attachment with nucleic acid-binding peptides by intra-branch electrostatic collapse of branches as depicted in the schematic. This unique mechanism and predetermined folding points of high flexibility provide a degree of control over complexation process with peptides, shifting aggregation/complexation from inter-particle aggregation to intraparticle aggregation which may be more entropically favorable while satisfying electrostatic charge neutralization of negative DNA charges with positive peptide charges.

FIGS. 10A to 10F show a unique ability of LL37 peptide sequence-complexed DNA branched origami NPs to remain stable and retained within alginate capsules as compared to K10 peptide sequence. FIG. 10A shows fluorescence microscopy image of alginate capsules loaded with FAM-K10OVA peptide complexed with Atto647-CpG-loaded origami, where K10-based OVA peptide was completely leaked out of the capsules immediately after manufacturing and while in the crosslinking buffer, CaCl₂).

FIG. 10B shows successful colocalization and retention of FAM-LL37 complexed with Atto647-CpG-loaded branches and encapsulated in alginate capsule as compared to alginate only control. Brightfield image of alginate capsule colocalized with the Atto647 and FAM signals for the alginate-encapsulated LL37/CpG/Branch. Interestingly, FIG. 10C shows that LL37 peptide was capable of being stably retained within alginate capsule on its own and when stored in PBS. FIG. 10D shows successful alginate encapsulation of FAM-LL37 complexed CpG-loaded branched origami in CaCl₂) buffer, as demonstrated by FAM signal colocalization (FAM fluorescence image) with alginate capsules (brightfield image). Furthermore, it is demonstrated that alginate capsules containing LL37/CpG/Branch/Origami were stable in CaCl₂) buffer, but not in 2.5 mM MgCl₂ buffer in PBS that is used to administer DNA origami or DNA branched origami formulations to cells or animals. In contrast, the same alginate capsules were stable in PBS for at least one day. FIG. 10E shows FAM fluorescence signal of agarose gel electrophoresis, demonstrating that DNA branched origami complexed FAM-LL37 peptides, as based on the shift of the band downward within the well as compared to free FAM-LL37 peptide shift upwards within the well. Alginate encapsulation of FAM-LL37 complexed branched origami was demonstrated by no shifting of the signal within the well. FIG. 10F shows TEM image of alginate-encapsulated LL37OVA and CpG-loaded DNA branched origami.

FIGS. 11A to 11C demonstrate resistance of LL37OVA complexed DNA branches and DNA branched origami to DNase degradation (7-hour exposure at 0.5 U/μL DNase I and 0.5 μg/μL DNA), and unexpected advantage of LL37 peptide complexation system to protect the attachment of phosphorothioated CpG oligonucleotides on DNA nanostructures as compared to K10 counterpart. FIG. 11A shows agarose gel electrophoresis signal corresponding to DNA structures on the left and Atto647 signal corresponding to CpG adjuvant on the right. It was seen that at suboptimal K10OVA coating of CpG-loaded origami, neither structures, nor the attachment of CpG to origami were preserved upon incubation with DNase (red arrow). In contrast, complexing CpG-loaded DNA nanostructures, in this case DNA branches, with LL37OVA peptides preserved the attachment of CpG on DNA nanostructures (red arrow). Please note that due to the exceptional CpG loading capacity of DNA branches based on mass of CpG DNA loaded per mass of branch DNA, and to maintain similar Atto647 fluorescence signal of CpG among conditions, the signal of DNA branches in ethidium bromide channel is not easily detectable, especially after complexation with LL37OVA, and similar is true for the free CpG signal. Alginate encapsulation of LL37OVA complexed branches also maintained resistance to DNase degradation (red arrow). FIG. 11B demonstrates that while K10OVA coating protected DNA origami structures from DNase as demonstrated by the presence of DNA origami band in well 2 and absence of the band in well 1 under DNase treatment (ethidium bromide signal), K10OVA did not protect the attachment of phosphorothioated CpG onto DNA origami nanoparticles from DNase as demonstrated by the lack of band in the well 2 (Atto647 signal, red arrow). In contrast, LL37OVA protected not only DNA branches and DNA branched origami, but also the attached phosphorothioated CpG payload (red arrows).

FIG. 11C shows TEM images demonstrating LL37OVA complexed CpG-loaded DNA branched origami structures had intact morphology after LL37OVA peptide coating and that LL37OVA protected structures from degradation.

FIGS. 12A to 12I demonstrate resistance of LL37OVA complexed DNA branches and DNA branched origami to degradation in 95% fetal bovine serum for various periods of time, and unexpected advantage of LL37 peptide complexation system to protect the attachment of phosphorothioated CpG oligonucleotides on DNA nanostructures as compared to K10 counterpart. The serum in these experiments was not heat-inactivated. FIG. 12A shows samples incubated with serum and immediately run on the gel as a reference representing no degradation. On the left is gel electrophoresis fluorescent image showing colocalization of CpG signal on DNA nanostructures in the wells as compared to free CpG signal (green arrows), while the gel on the right shows peptide signal (green square shows K10OVA peptide migrating in the opposite direction from DNA structures). FIG. 12B shows samples incubated with serum for 7 hours. The gel on the left shows Atto647 signal demonstrating that FAM-K10OVA did not help protect or retain CpG on the origami NP, while FAM-LL37 helped retain some CpG on the branched origami NPs (green arrows). Alginate fully protected CpG on branched DNA dendrimers complexed with FAM-LL37 peptides. The gel on the right shows FAM signal, demonstrating peptides remained complexed with DNA structures, and that DNA structures protected K10 or LL37 based peptides (red squares indicate degraded peptide components). Interestingly, LL37 peptides on their own (i.e., without complexation with DNA structures) were stable within alginate capsules and did not leak or degrade even after 7 hours of incubation with serum. FIG. 12C shows another set of samples incubated with serum for 7 hours. The main difference in this gel as compared to gel in FIG. 12B is that non-FAM LL37OVA was shown to provide full protection of and/or compatibility with phosphorothioated CpG attachment (green arrows, wells 13 and 14) as compared to FAM-LL37 or FAM-K10OVA. FIG. 12D shows gel for the samples incubated with serum for 48 hours. The gel on the left shows Atto647 signal demonstrating that FAM-K10OVA did not help protect or retain CpG on the origami NP, while FAM-LL37 helped retain some CpG on the branched origami NPs (green arrows). Alginate fully protected CpG on branched DNA dendrimers complexed with FAM-LL37 peptides (green arrow), while some CpG-loaded branches were pulled out from alginate bead during gel electrophoresis (green square), demonstrating alginate beads sequestered formulations from interaction with serum for at least 48 hours. The gel on the right shows FAM signal demonstrating peptides completely degraded after 48 hours in serum (red squares), except FAM-LL37 peptide which appeared to have a low level of degradation. Unexpectedly, branched DNA dendrimers protected LL37 as compared to LL37 alone. Again, alginate encapsulation fully protected LL37 peptides complexed with CpG-loaded DNA branches. Interestingly, LL37 peptide was protected from degradation and stable within alginate capsule even after 48 hours. FIG. 12E is a TEM image demonstrating that the majority of CpG-loaded DNA origami were degraded after degradation in serum for 7 hours. FIGS. 12F to 12I are TEM images demonstrating different levels of DNA NP degradation after 7 hours in serum, with many intact structures visible for LL37OVA coated CpG-loaded DNA branched origami structures.

FIG. 13 demonstrates successful purification of endotoxin down to 2 EU/g or below for p7249 scaffolds and DNA structures used as components of vaccines. To note is that alginate used in this project was low endotoxin alginate.

FIGS. 14A to 14C are confocal fluorescence images demonstrating binding and uptake of CpG-loaded DNA branched origami complexed with K10- or LL37-based peptides in peritoneal macrophages isolated from C57/BI6 WT mice. Cells were plated on gelatin-coated coverslips as a mimic of extracellular matrix, and upon attachment, formulations were administered in complete medium containing 10% heat inactivated fetal bovine serum. FIG. 14A demonstrate that LL37 complexed DNA structures, either CpG-loaded DNA branches or DNA branched origami, bound only to cells and not the gelatin-coated coverslips as compared to PBS control. Although incubated at 4° C., where active cell uptake is prevented, a profuse cytosolic signal was still detected for both, the peptide and CpG oligonucleotide components for both, DNA branches and DNA branched origami structures complexed with LL37 peptide. Images also demonstrated colocalization of peptide and oligonucleotide components. FIG. 14B compares LL37- and K10-based peptide attachment system to DNA branches after 30 minutes of incubation at 37° C. While both systems resulted in oligonucleotide signal located on cells only, and some colocalization with peptide signal, only LL37-based attachment resulted in peptide delivery specifically to cells, while K10-based attachment resulted also in non-specific binding to gelatin-coated coverslips. FIG. 14C compares LL37- and K10-based peptide attachment system to DNA branched origami. Cells were incubated with formulations for 5 hours (pulse), and then media replaced with fresh media lacking formulations for 3 hours (chase) at 37° C. (8 hours total incubation time). While both systems resulted in oligonucleotide signal located on cells only, and formulations were internalized based on their location within the cells, again, only LL37-based attachment resulted in peptide delivery specifically to cells, while K10-based attachment resulted also in non-specific binding of peptides to gelatin-coated coverslips. To note is that some punctuate signal was also seen on the coverslips for LL37 complexed DNA NP formulation, but here the peptide and oligonucleotide signals were colocalized as compared to K10-based peptide signal which was profuse throughout the whole coverslip appearing as free peptide; some level of NP binding to gelatin-coated coverslips is expected.

FIG. 15 is a confocal fluorescence image demonstrating colocalization of CpG-loaded DNA branched origami with phagolysosomes or lysosomes and cytosolic delivery ability. MUTUDC1940 dendritic cell line was plated on gelatin-coated coverslips, cells were pulsed with dextran (shown in red) for 45 minutes, media replaced for 45 minutes to allow accumulation of dextran in lysosomes or phagolysosomes, and then treated with PBS, CpG, or CpG-loaded DNA branched origami. Images demonstrate that CpG-loaded DNA branched origami does not hinder delivery of CpG to lysosomes or phagolysosomes, or cytosolic escape of oligonucleotides (e.g., CpG).

FIG. 16 shows flow cytometry results demonstrating enhanced uptake of peptide antigens and phosphorothioated CpG oligonucleotide adjuvants for various DNA NP-based delivery systems utilizing or not K10- and LL37-based peptide attachment technology administered to professional antigen presenting cells in splenocyte co-culture (after isolation from C57/BI6 WT mice and red blood cell lysis and removal). Complete media containing 10% heat inactivated fetal bovine serum was used. Please note that the OVA here stands for SIINFEKL peptide, K10OVA stands for KKKKKKKKKKSIINFEKL, and LL37 stands for human LL37, LLGDFFRKSKEKIGKEFKRIVQRIKDFLRNLVPRTES. The graph on the left demonstrates that both regular origami and branched origami increased delivery of peptides to B cells, while the graph on the right demonstrates that unexpectedly DNA branched origami strongly enhanced the delivery of CpG as compared to DNA branch or DNA origami counterparts, although all three formulations (i.e., DNA branches, DNA origami, and DNA branched origami) enhanced the delivery of CpG as compared to free CpG. Interestingly, K10-based attachment reduced phosphorothioated CpG delivery, particularly in macrophage and dendritic cell populations, while LL37 caused no difference in macrophage and dendritic cell populations, but appeared to enhance delivery in B cells. (Data were calculated as mean±standard deviation. Significance was determined using ANOVA followed by Tukey's test, assuming a p-value of 0.05.)

FIG. 17 shows flow cytometry results demonstrating enhanced peptide antigen presentation via MHCI and activation via upregulation of co-stimulatory molecules in MUTUDC1940 dendritic cell line by DNA branch- and DNA branched origami-based vaccine formulations incubated in complete media containing 10% heat inactivated fetal bovine serum. Please note that the OVA here stands for SIINFEKL peptide, K10OVA stands for KKKKKKKKKKSIINFEKL, and LL37OVA stands for LLGDFFRKSKEKIGKEFKRIVQRIKDFLRNLVPRTESSIINFEKL, where LL37 portion of this chimeric peptide is a human version of the sequence. Top left figure demonstrates enhanced antigen presentation of K10-based OVA peptide complexation with CpG-loaded DNA branched origami as compared to OVA peptide alone. Please note that flow cytometry antibody was used that specifically detects SIINFEKL peptide complexed with MHCI, but not SIINFEKL peptide alone or bound elsewhere on cell or NP surface. Top right figure demonstrates MHCII upregulation by all formulations containing CpG, but not DNA origami alone. Bottom left figure demonstrates that all CpG-containing formulations induced upregulation of CD40 co-stimulatory molecule as compared to PBS control, LL37OVA peptide, or DNA origami alone. Furthermore, it demonstrates that although LL37OVA peptide attachment technology did not stimulate dendritic cells by itself, it enhanced dendritic cell stimulation of CpG-loaded DNA branches. It also demonstrates that DNA branching design of DNA origami also enhanced dendritic cell stimulation as compared to CpG alone, and this was further enhanced by LL37- or K10-based peptide attachment system. Importantly, this figure also demonstrates advantage of CpG-loaded DNA branched origami design stimulation over CpG-loaded DNA branch design alone. Bottom right figure demonstrates similar enhancements for upregulation of co-stimulatory molecule CD80 as for CD40. (Data were calculated as mean±standard deviation. Significance was determined using ANOVA followed by Tukey's test, assuming a p-value of 0.05.)

FIGS. 18A and 18B show flow cytometry results demonstrating peptide antigen presentation via MHCI and activation via upregulation of co-stimulatory molecules induced by various DNA NP-based delivery systems utilizing or not K10- and LL37-based peptide attachment technology administered to professional antigen presenting cells in mouse splenocyte co-culture (after isolation from C57/BI6 WT mice and red blood cell lysis and removal). Complete media containing 10% heat inactivated fetal bovine serum was used. Please note that the OVA here stands for SIINFEKL peptide, K10OVA stands for KKKKKKKKKKSIINFEKL, and LL37OVA stands for LLGDFFRKSKEKIGKEFKRIVQRIKDFLRNLVPRTESSIINFEKL, where LL37 portion of this chimeric peptide is a human version of the sequence. FIG. 18A demonstrates successful antigen processing of K10- and LL37-based antigen peptides. It also demonstrates the advantage of using DNA branched origami-induced peptide delivery to enhance antigen presentation as compared to DNA branch or DNA origami counterparts. Please note that LL37-based antigen peptide delivery resulted in lower SIINFEKL presentation as compared to the native SIINFEKL, and this may have been either due to inherent peptide design differences or differences in peptide solubility and suboptimal dissolution/reconstitution of peptides due to the inexperience and novelty of LL37-based chimeric polypeptides which behave very differently than LL37 peptides alone or SIINFEKL peptides alone. FIG. 18B demonstrates upregulation of co-stimulatory molecules CD40 and CD86, where CpG-loaded DNA branched origami design was advantageous in stimulating CD40 in macrophages and dendritic cells as compared to CpG-loaded DNA branch design and CpG alone, while DNA branches did not stimulate cells and were found to be non-immunogenic. DNA origami itself did stimulate cells and was found to be immunogenic. Due to complexity of showing statistics in this graph, statistics are shown only comparing CpG-loaded DNA branched origami vs. all other conditions. (Data were calculated as mean±standard deviation. Significance was determined using ANOVA followed by Tukey's test, assuming a p-value of 0.05.)

FIG. 19 shows biodistribution of Atto647-CpG-loaded and LL37OVA coated DNA branches as compared to of Atto647-CpG alone after I.M. injection in albino C57/B16 mice using IVIS imaging. Mouse a and b were imaged in different positions before injections to demonstrate background signal and subsequently injected with either Atto647-CpG-loaded DNA branches complexed with LL37OVA peptide (mouse a), or Atto647-CpG alone (mouse b). Images were taken immediately or 20 hours after injection. It was noticed, similar as with fluorescence gel imaging that Atto647 signal on CpG was attenuated by LL37OVA peptide coating. Hence, the whole-body images demonstrate stronger initial signal (immediately after injection) for CpG alone as compared to the DNA branch-based vaccine, despite injection of the same Atto647-CpG dose. However, 20 hours after injection, the whole-body images demonstrate a very strong signal for the Atto647-CpG-loaded DNA branches complexed with LL37OVA peptide (mouse a), while the signal for the Atto647-CpG alone is very weak. In addition, the signal for the Atto647-CpG-loaded DNA branches complexed with LL37OVA peptide (mouse a) is very localized and appears to be completely in the lymph node, while the signal for Atto647-CpG alone (mouse b) is more diffused. Indeed, after collection of the draining lymph nodes (top) as compared to non-draining lymph nodes (bottom), it was verified that the formulations were specifically delivered to the draining lymph nodes as intended. On the other hand, it was observed that the K10-based peptide delivery system takes at least 3 days to deliver DNA NP-based formulations to draining lymph nodes, perhaps due to very strong non-specific binding to tissues (data not shown). Please note that OVA indicates SIINFEKL in these figures.

FIG. 20 shows flow cytometry results demonstrating that K10OVA-complexed and CpG-loaded DNA branched origami enhanced SIINFEKL-specific T cell expansion and activation, as compared to mixture of OVA+CpG or PBS buffer control, 3 days after injection of indicated samples via S.C. route in C57/B16 WT mice that previously received adoptive transfer of SIINFEKL specific- and Cell Trace Violet-labeled T cells from OT-1 mice. This was despite OVA being at double the dose in the OVA+CpG mixture as compared to DNA branched origami vaccine (graph on the left). From the kinetics of the T cell expansion, it was evident that DNA branched origami vaccine delivery technology has delayed and prolonged immune stimulation (graph on the left). CD25 activation marker on T cells demonstrated that this stimulation lasted for at least 3 days, and was significantly enhanced as compared to the OVA+CpG mixture or PBS buffer control (graphs in the middle and on the right). This in part may have been due to K10-induced nonspecific tissue interactions as mentioned above in the biodistribution study (FIG. 19). Please note that OVA indicates SIINFEKL in these figures. (Data were calculated as mean±standard deviation. Significance was determined using ANOVA followed by Tukey's test, assuming a p-value of 0.05.)

FIG. 21 shows flow cytometry results demonstrating enhanced antigen-specific target cell killing induced by CpG-loaded DNA branched DNA origami or DNA branches complexed with either K10- or LL37-based SIINFEKL delivery systems via various administration routes. A standard in vivo T cell cytotoxic killing assay was used. Briefly, in these experiments, C57/BI6 WT mice were vaccinated with one vaccinr dose (prime vaccination) or two vaccine doses separated 2 weeks apart (prime+boost vaccination) or administered PBS, as indicated in the graphs, followed by I.V. injection of differentially stained target (SIINFEKL-pulsed and Cell Trace Violet low signal intensity-labeled) and non-target (un-pulsed and Cell Trace Violet high signal intensity-labeled) splenocytes (after red blood cell lysis and removal) 7 days post last vaccination. 24 hours later, spleens were isolated and red blood cells removed to measure the two Cell Trace Violet-labeled cell populations (i.e., the high and the low Cell Trace Violet fluorescence signal intensity populations). The percent killing was calculated using standard equations, based on the target and non-target populations in each condition and in reference to control PBS (untreated) mice where no antigen specific killing is present. Percentages of the two populations (high and low fluorescence intensity populations) were also verified each time before I.V. injection. Top left figure demonstrates that CpG-loaded and K10SIINFEKL-complexed DNA branched origami, upon a single S.C. dose, induced significantly higher antigen-specific target cell killing as compared not only to the mixture of CpG+OVA or mixture of CpG+OVA+DNA origami, but also gold standard subunit vaccine references based on the Complete Freund's Adjuvand (CFA) and alum. Alum is currently used in many human vaccines, while CFA is not used in humans due to reactogenicity. The DNA branched origami formulation with a scramble version of K10OVA peptide did not induce killing as expected. All formulations presented in this graph were S.C. and single injections. Top right figure demonstrates that, upon a single dose via I.M. route, LL37-based SIINFEKL attachment to either CpG-loaded DNA branches or DNA branched origami induced significantly higher antigen-specific target cell killing as compared to the mixture of OVA+CpG or LL37 control peptide complexed with CpG-loaded DNA branched origami formulation. Surprisingly, the bottom left figure demonstrates that LL37-based attachment technology was suitable and capable, when complexed to CpG-loaded DNA branches to induce significant antigen-specific T cell killing of target cells via I.V. route vaccination. Furthermore, the prime+boost regimen resulted in improved and more consistent responses as compared to prime only vaccination for I.M. injected LL37OVA-complexed CpG-loaded DNA branches. The bottom right figure shows prime, as well as prime+boost vaccination for a regular CpG-loaded origami complexed with K10OVA peptides, demonstrating that significant improvement of this formulation over the mixture of CpG+OVA was achieved only after the prime+boost dose. This result indicates enhanced performance of DNA branched origami and LL37-peptide attachment technology. All the formulations contained the dose per each mouse, as applicable, 0.01 nmol DNA origami, 0.64 nmol CpG, and 7.25 nmol antigenic peptide. CFA was prepared by mixing the ratio of 7.25 nmol SIINFEKL in 50 μL PBS with 50 μL CFA; larger quantity was prepared and properly emulsified to allow drawing of 100 μL in each syringe; it was verified that the emulsion was stable at least for several months by setting aside an aliquot and observing over time. Alum-based vaccine was prepared by mixing 7.25 nmol SIINFEKL in 50 μL PBS with 50 μL of 2% aluminum hydroxide (Alhydrogel). (Data were calculated as mean±standard deviation. Significance was determined using ANOVA followed by Tukey's test, assuming a p-value of 0.05.)

FIG. 22 shows therapeutic anti-tumor efficacy of LL37-gp100 complexed with CpG and enhanced therapeutic efficacy of alginate capsules containing LL37gp100-complexed and CpG-loaded DNA branches against B16-F10 melanoma tumor model in C57/B16 WT mice. Mice were injected S.C. with 2.5×10⁵ B16-F10 cells in 50 μL of PBS on day 0, weighed and monitored for tumor growth. On day 3, one group (shown in green, LL37gp100/CpG group) of mice was vaccinated via I.M. route with LL37gp100 complexed with CpG; the dose of CpG was 1.28 nmol/mouse and 20 nmol LL37gp100 peptide. On days 8 and 9, intratumoral injections were performed as indicated: LL37gp100/CpG group received LL37gp100/CpG, while Alginate Capsule/LL37gp100/CpG/Branch group received Alginate Capsule/LL37gp100/CpG/Branch, where the dose for each injection was 0.64 nmol CpG and 10 nmol LL37gp100 per each mouse. Please note that the two subsequent intratumoral injections were performed, where the dose was split between the two days, due to the difficulty of intratumoral injections using peptide volumes as reconstituted in this study. Figure on the left demonstrates surprising and rapid anti-tumor efficacy of Alginate Capsule/LL37gp100/CpG/Branch formulation upon only 2 subsequent doses, which was significantly better than the efficacy of LL37gp100/CpG complex which was administered as 3 total doses (4 dose equivalent to the alginate formulation) or PBS/untreated mice. Both, LL37gp100/CpG complex and Alginate Capsule/LL37gp100/CpG/Branch demonstrated significant reduction in tumor growth. Figure on the right demonstrates improved survival for both the LL37gp100/CpG complex and Alginate Capsule/LL37gp100/CpG/Branch formulations as compared to PBS/untreated mice. (Data were calculated as mean±standard deviation. Significance was determined using ANOVA followed by Tukey's test, assuming a p-value of 0.05.)

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

As used herein, the terms “optional” or “optionally” means that the subsequently described event or circumstance can or can not occur, and that the description includes instances where said event or circumstance occurs and instances where it does not.

As used herein, the term “subject” refers to the target of administration, e.g., an animal. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a patient. A patient refers to a subject afflicted with a disease or disorder, such as, for example, cancer and/or aberrant cell growth. The term “patient” includes human and veterinary subjects. In an aspect, the subject has been diagnosed with a need for treatment for cancer and/or aberrant cell growth.

The terms “treating”, “treatment”, “therapy”, and “therapeutic treatment” as used herein refer to curative therapy, prophylactic therapy, or preventative therapy. As used herein, the terms refers to the medical management of a subject or a patient with the intent to cure, ameliorate, stabilize, or prevent a disease, pathological condition, or disorder, such as, for example, cancer or a tumor. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In an aspect, the disease, pathological condition, or disorder is cancer, such as, for example, breast cancer, lung cancer, colorectal, liver cancer, or pancreatic cancer. In an aspect, cancer can be any cancer known to the art.

As used herein, the terms “administering” and “administration” refer to any method of providing a composition to a subject. Such methods are well known to those skilled in the art and include, but are not limited to, intratumoral administration, intracardiac administration, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed composition or peptide or pharmaceutical preparation and a cell, target receptor, or other biological entity together in such a manner that the compound can affect the activity of the target (e.g., receptor, transcription factor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, in an aspect, an effective amount of the polymeric nanoparticle is an amount that kills and/or inhibits the growth of cells without causing extraneous damage to surrounding non-cancerous cells. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts.

The term “pharmaceutically acceptable” describes a material that is not biologically or otherwise undesirable, i.e., without causing an unacceptable level of undesirable biological effects or interacting in a deleterious manner. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

The term “DNA origami” refers to nanoscale folding of DNA to create non-arbitrary two- and three-dimensional shapes at the nanoscale. DNA origami works by using a long “scaffold” strand of DNA and holding it together using many short “staple strands.”

### DNA Branch Nanostructure

Details on how to design and produce DNA branched dendritic structures can be found in Nilsen, et al. Dendritic Nucleic Acid Structures. Journal of theoretical biology 187.2 (1997): 273-284 and in Roki, et al. Unprecedently high targeting specificity toward lung ICAM-1 using 3DNA nanocarriers. Journal of Controlled Release 305 (2019): 41-49.

### DNA Origami Nanostructure

Details on how to design and produce DNA origami nanostructures can be found in Castro, et al. A primer to scaffolded DNA origami. Nature methods, 8 (3), 221-229. which is incorporated by reference herein for these teachings. DNA design software, such as caDNAno, can be used to design 3D DNA origami nanostructures as disclosed herein and described in Glaser, et al. The Art of Designing DNA Nanostructures with CAD Software. Molecules 26.8 (2021): 2287. Similar DNA origami design modified as an example of DNA origami NP used in compositions here is described in Halley P D, et al. Daunorubicin-Loaded DNA Origami Nanostructures Circumvent Drug-Resistance Mechanisms in a Leukemia Model. Small. 2016 12 (3): 308-20.

### LL37 Peptide

LL37 peptide is endogenous antimicrobial peptide with natural ability to bind DNA, among other molecules. Many and complex roles have been described for this peptide, which is currently under extensive research regarding its mechanisms, behavior and interactions with biological environment, particularly cell membranes, as well as its implications in health and diseases. LL37 is extensively being explored as a potential therapeutic in various applications such as wound healing, immunomodulation, tumor treatment, etc. Details can be found in Chamilos, et al. Cytosolic sensing of extracellular self-DNA transported into monocytes by the antimicrobial peptide LL37. The Journal of the American Society of Hematology 120.18 (2012): 3699-3707.

Disclosed herein are unique and unexpected properties of LL37 that other DNA-binding peptides via electrostatic interactions do not seem to possess, especially in regards to DNA NP complexation with LL37, specifically 3 properties:

As disclosed herein, there are incredible stability and retention properties of LL37 within alginate. LL37 and LL37 chimeric polypeptides, whether complexed with DNA NPs or not, have been found to remain within alginate capsules (LL37 does not leak, and remains in alginate even after 48 h of degradation in FBS or DNase, this is crazy!), while for example K10 leaks from the alginate capsules immediately after capsule formation; also, in general, peptides leak from alginate capsules within hours. Therefore LL37 has a unique potential for controlled release delivery in combination with alginate. Whether this is true for other gel systems other than alginate and other materials is not known. Therefore, so far, the LL37 chimeric peptides are the only candidates that have the ability to stay complexed with DNA NPs and at the same time be retained in alginate capsules. For K10, DNA NPs can stay in the alginate capsules, but the peptide leaks out immediately, so antigen and adjuvant can not be co delivered together in that case, negating the whole point of using NP-based delivery system.

As disclosed herein, there is an absence of non-specific binding of LL37, which is key for DNA NP delivery. It is expected that any molecule engaging in electrostatic binding will not have much specificity of what it binds to, especially when the zeta potential is brought close to 0 and NPs start to aggregate in addition to sticking to everything. But LL37 is different than other peptides in this regard, and most likely it is different than all the other positively charged peptides. Therefore, it is seen in fluorescence images that LL37 complexed with DNA NPs is bound to cells only and not the coverslips coated with gelatin. In contrast, lysine is commonly used to coat tissue culture plates to enhance cell attachment. Because of this reason, K10 causes non-specific binding to most of the things, such as gelatin-coated coverslips as seen in fluorescence images, as well as to the filters (I spent almost 3 months only trying to quantify K10SIINFEKL attachment to origami, but this was impossible since K10 sticks to everything!). Furthermore, upon injection, it takes for at least 3 days till DNA NPs bound to K10 can even reach the lymph nodes because K10 sticks to everything as soon as it is injected. In contrast, DNANPs are delivered within 20 hours to lymph nodes when using LL37 system. This is the precise reason why I couldn't make the DNA origami combined with K10 work in vivo until we switched from S.C. to I.M. injection; the K10 most likely binds to fat cells upon S.C. injection, preventing delivery where it is supposed to go, the lymph nodes. The branched origami design prevents the majority of the K10-induced DNA NP aggregation and works even via S.C. injection. But S.C. is not a good route for K10 system in general, and I.V. route as currently designed is not possible best to my knowledge and the testing that I have done.

Also disclosed herein is DNANP/LL37 I.V. delivery. The property of LL37 described above is most likely one of the reasons why LL37 is suitable for I.V. vaccination and K10 is not. In general, positively charged things can not do well via I.V. So the fact that LL37 bound DNA NPs can accomplish immunization via I.V. is a “miracle”, despite LL37 having a charge of +6. The points 2 and 3 go hand in hand with each other. Also, has there ever been a DNA-based or DNA-made NP that successfully induced vaccination, and specifically T cell-based immunization via the I.V. route? How many vaccine technologies can do this? Liposomes, liposomes/mRNA, perhaps naked mRNA, viral-like NPs, and viruses, attenuated or native ones. But to achieve it with peptide antigen, such as SIINFEKL, how many technologies managed that via I.V. route?

So given all these 3 properties unique to LL37 and DNANPs, DNA NPs can “properly” be delivered when bound to LL37, where the electrostatic attachment, which is actually detrimental to the performance and properties of DNA NPs, is not so severe for LL37 system as compared to for example K10 and others tested so far. While several researchers and patents broadly mention or broadly claim LL37 chimeric peptides bound to nanoparticles or mentioned LL37-PEG bound to DNA NPs in their patents, no one has actually tested and discovered these 3 points, and these claims were simply made based on the general knowledge that LL37 binds DNA. The LL37/alginate combo is a completely new concept and phenomenon, best to my knowledge. Since no one has tested and discovered these 3 things despite the common knowledge about DNA+LL37, may be proof in itself that this is worthy of being granted these claims, and demonstrates non-obviousness. The property of LL37 to avoid non-specific binding may be more obvious, but LL37 chimeric peptides may still be able to be treated as a new entity for which these properties could not have been predicted to be similar as for native LL37.

### Antigens

Peptide antigens are described, for example, in Abd-Aziz, N, et al. J Oncol. 2022 2022:9749363, and Buonaguro, et al. Vaccines (Basel). 2020 8 (4): 615, which are incorporated by reference in their entireties for the teaching of these antigens and their uses as vaccines. For example, in some embodiments, the antigen is a tumor antigen, such as HER-2, hTERT, mesothelin, MUC-1, p53, gp100, MART-1, PSA, PAP, tyrosinase, BAGE, MAGE, GAGE, PRAME, NY-ESO-1, EBV LMP-1/LMP-2A, HPV-E6/E7, HTLV-1, KRAS, NRAS, epitopes from BCR-ABL translocation, ETV6, NPM/ALK, or ALK. In some embodiments, the antigen is Nelipepimut-S(NP-S), MDX-1379 (gp100), MAGE-A3/NY-ESO-1, or G17DT.

In some embodiments, the tumor antigen is an antigen for acute lymphoblastic leukemia (ALL), Breast Cancer, Fibrolamellar hepatocellular carcinoma (HCC), Follicular Lymphoma, Gastric Cancers, Glioblastoma, hepatocellular carcinoma (HCC), Kidney Cancer, Lymphocytic Leukemia, Melanoma, non-small cell lung cancer (NSCLC), Ovarian Cancer, Pancreatic Cancer, Pediatric Brain Tumor, Prostate Cancer, small cell lung cancer (SCLC), smoldering plasma cell myeloma (SPCM), triple-negative breast carcinoma (TNBC), or urothelial/bladder cancer (UBC).

In some embodiments, the antigen is a human endogenous retroviral element (HERV). HERV-derived antigens have been used to develop cancer vaccines and chimeric antigen receptor (CAR)-expressing T cells, and can be adapted for use in the disclosed compositions and methods.

In some embodiments, the antigen is encoded as an mRNA antigen, such as a viral antigen. Therefore, in some embodiments, the antigen is a viral antigen. For example, in some embodiments, the virus is an influenza A, an influenza B, a cytomegalovirus (CMV), respiratory syncytial virus (RSV), coronavirus (e.g. SARS-COV-2), human papillomavirus (HPV), varicella, dengue, diptheria, ebola, hepatitis, human immunodeficiency virus (HIV), encephalitis, measles, monkeypox, mumps, norovirus, polio, rabies, rotavirus, rubella, herpes, or zika virus.

Viral antigens are described, for example, in Pollard, A J, et al. Nature Reviews Immunology 2021 21:83-100, Kyriakidis, N C, et al. NPJ Vaccines. 2021 6 (1): 28; and Andrei, G, et al. Front. Virol., May 24 2021, which are incorporated by reference in their entireties for the teaching of these viral antigens and uses in vaccines.

In some embodiments, the antigen is a β-amyloid (AB) or Tau peptide for production of an Alzheimer's Disease vaccine. Examples of peptide antigens are descriped in Malonis, R J, et al. Chem Rev. 2020 120 (6): 3210-3229, which is incorporated by reference in its entirety for the teaching of these peptides and their uses as a vaccine.

### Alginate

Alginates are naturally occurring polysaccharide biopolymers extensively used in biomedical applications. Alginate can be crosslinked using divalent cations such as Ca²⁺. Details can be found in Lee, at al. Alginate: Properties and biomedical applications. Progress in polymer science 37.1 (2012): 106-126.

Pharmaceutical Formulations Also provided herein are pharmaceutical formulations that can include an amount of a DNA branch or DNA branched origami nanostructures, LL37 peptide chimeras, all incorporated or not within alginate capsules, described herein and a pharmaceutical carrier appropriate for administration to an individual in need thereof. The individual in need thereof can have or can be suspected of a cancer, a genetic disease or disorder, a viral, bacterial, fungal, and/or parasitic infection, or other disease or disorder in need of treatment or prevention. In some embodiments, the subject in need thereof is in need of a diagnostic procedure, such as an imaging procedure. The pharmaceutical formulations can include an amount of a disclosed DNA or DNA/chimeric polypeptide nanostructures, encapsulated in alginate, or not, such as that they can be effective to treat or prevent a cancer, a genetic disease or disorder, a viral, bacterial, fungal, and/or parasitic infection, or other disease or disorder or be effective to image the subject or a portion thereof. Formulations can be administered via any suitable administration route. For example, the formulations (and/or compositions) can be administered to the subject in need thereof orally, intravenously, ocularly, intraocularly, intramuscularly, intravaginally, intraperitoneally, rectally, parenterally, topically, intranasally, or subcutaneously. Other suitable routes are described herein. In some embodiments, the disclosed DNA nanostructure-based formulations contain an effective amount of a cargo molecule.

### Parenteral Formulations

The disclosed DNA nanostructures, complexed with peptides or not, and encapsulated in alginate capsules or not, and LL37 chimeric peptides, can be formulated for parenteral delivery, such as injection or infusion, in the form of a solution or suspension. The formulation can be administered via any route, such as, the blood stream or directly to the organ or tissue to be treated.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the disclosed DNA nanostructures can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants can be anionic, cationic, amphoteric or nonionic surface active agents. Suitable anionic surfactants include, but are not limited to, those containing carboxylate, sulfonate and sulfate ions. Suitable anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Suitable cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Suitable nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation can also contain an antioxidant to prevent degradation of the disclosed DNA-based nanostructures.

The formulation can be buffered to a pH of 3-8 for parenteral administration upon reconstitution. Suitable buffers include, but are not limited to, phosphate buffers, acetate buffers, and citrate buffers.

Water-soluble polymers can be used in the formulations for parenteral administration. Suitable water-soluble polymers include, but are not limited to, polyvinylpyrrolidone, dextran, carboxymethylcellulose, and polyethylene glycol. Sterile injectable solutions can be prepared by incorporating the disclosed DNA-based nanostructures in the required amount in the appropriate solvent or dispersion medium with one or more of the excipients listed above, as required, followed by filtered sterilization. Dispersions can be prepared by incorporating the various sterilized disclosed DNA-based nanostructures into a sterile vehicle which contains the basic dispersion medium and the required other ingredients from those listed above. Sterile powders for the preparation of sterile injectable solutions can be prepared by vacuum-drying and freeze-drying techniques, which yields a powder of the disclosed DNA-based nanostructures plus any additional desired ingredient from a previously sterile-filtered solution thereof. The powders can be prepared in such a manner that the particles are porous in nature, which can increase dissolution of the particles. Methods for making porous particles are well known in the art.

Pharmaceutical formulations for parenteral administration can be in the form of a sterile aqueous solution or suspension of particles formed from one or more disclosed DNA-based nanostructures. Acceptable solvents include, for example, water, Ringer's solution, phosphate buffered saline (PBS), and isotonic sodium chloride solution. The formulation can also be a sterile solution, suspension, or emulsion in a nontoxic, parenterally acceptable diluent or solvent such as 1,3-butanediol.

In some instances, the formulation can be distributed or packaged in a liquid form. In other embodiments, formulations for parenteral administration can be packed as a solid, obtained, for example by lyophilization of a suitable liquid formulation. The solid can be reconstituted with an appropriate carrier or diluent prior to administration.

Solutions, suspensions, or emulsions for parenteral administration can be buffered with an effective amount of buffer necessary to maintain a pH suitable for ocular administration. Suitable buffers include, but are not limited to, acetate, borate, carbonate, citrate, and phosphate buffers.

Solutions, suspensions, or emulsions for parenteral administration can also contain one or more tonicity agents to adjust the isotonic range of the formulation. Suitable tonicity agents include, but are not limited to, glycerin, mannitol, sorbitol, sodium chloride, and other electrolytes.

Solutions, suspensions, or emulsions for parenteral administration can also contain one or more preservatives to prevent bacterial contamination of the ophthalmic preparations. Suitable preservatives include, but are not limited to, polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions, use of nanotechnology including nanoformulations for parenteral administration can also contain one or more excipients, such as dispersing agents, wetting agents, and suspending agents.

### Topical Formulations

The disclosed DNA-based nanostructures can be formulated for topical administration. Suitable dosage forms for topical administration include creams, ointments, salves, sprays, gels, lotions, emulsions, liquids, and transdermal patches. The formulation can be formulated for transmucosal, transepithelial, transendothelial, or transdermal administration. The topical formulations can contain one or more chemical penetration enhancers, membrane permeability agents, membrane transport agents, emollients, surfactants, stabilizers, and combination thereof.

In some embodiments, the disclosed DNA-based nanostructures can be administered as a liquid formulation, such as a solution or suspension, a semi-solid formulation, such as a lotion or ointment, or a solid formulation. In some embodiments, the disclosed DNA-based nanostructures can be formulated as liquids, including solutions and suspensions, such as eye drops or as a semi-solid formulation, such as ointment or lotion for topical application to the skin, to the mucosa, such as the eye, to the vagina, or to the rectum.

The formulation can contain one or more excipients, such as emollients, surfactants, emulsifiers, penetration enhancers, and the like.

Suitable emollients include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In some embodiments, the emollients can be ethylhexylstearate and ethylhexyl palmitate.

Suitable surfactants include, but are not limited to, emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In some embodiments, the surfactant can be stearyl alcohol.

Suitable emulsifiers include, but are not limited to, acacia, metallic soaps, certain animal and vegetable oils, and various polar compounds, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In some embodiments, the emulsifier can be glycerol stearate.

Suitable classes of penetration enhancers include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols).

Suitable emulsions include, but are not limited to, oil-in-water and water-in-oil emulsions. Either or both phases of the emulsions can include a surfactant, an emulsifying agent, and/or a liquid non-volatile non-aqueous material. In some embodiments, the surfactant can be a non-ionic surfactant. In other embodiments, the emulsifying agent is an emulsifying wax. In further embodiments, the liquid non-volatile non-aqueous material is a glycol. In some embodiments, the glycol is propylene glycol. The oil phase can contain other suitable oily pharmaceutically acceptable excipients. Suitable oily pharmaceutically acceptable excipients include, but are not limited to, hydroxylated castor oil or sesame oil can be used in the oil phase as surfactants or emulsifiers.

Lotions containing a disclosed DNA-based nanostructures are also provided. In some embodiments, the lotion can be in the form of an emulsion having a viscosity of between 100 and 1000 centistokes. The fluidity of lotions can permit rapid and uniform application over a wide surface area. Lotions can be formulated to dry on the skin leaving a thin coat of their medicinal components on the skin's surface.

Creams containing a disclosed DNA-based nanostructures as described herein are also provided. The cream can contain emulsifying agents and/or other stabilizing agents. In some embodiments, the cream is in the form of a cream having a viscosity of greater than 1000 centistokes, typically in the range of 20,000-50,000 centistokes. Creams, as compared to ointments, can be easier to spread and easier to remove.

One difference between a cream and a lotion is the viscosity, which is dependent on the amount/use of various oils and the percentage of water used to prepare the formulations. Creams can be thicker than lotions, can have various uses, and can have more varied oils/butters, depending upon the desired effect upon the skin. In some embodiments of a cream formulation, the water-base percentage can be about 60% to about 75% and the oil-base can be about 20% to about 30% of the total, with the other percentages being the emulsifier agent, preservatives and additives for a total of 100%.

Ointments containing a disclosed DNA-based nanostructures as described herein and a suitable ointment base are also provided. Suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

Also described herein are gels containing a disclosed DNA-based nanostructures as described herein, a gelling agent, and a liquid vehicle. Suitable gelling agents include, but are not limited to, modified celluloses, such as hydroxypropyl cellulose and hydroxyethyl cellulose; carbopol homopolymers and copolymers; thermoreversible gels and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents can be selected for their ability to dissolve the drug. Other additives, which can improve the skin feel and/or emolliency of the formulation, can also be incorporated. Such additives include, but are not limited, isopropyl myristate, ethyl acetate, C₁₂-C₁₅ alkyl benzoates, mineral oil, squalane, cyclomethicone, capric/caprylic triglycerides, and combinations thereof.

Also described herein are foams that can include a disclosed DNA-based nanostructures as described herein. Foams can be an emulsion in combination with a gaseous propellant. The gaseous propellant can include hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or can become approved for medical use are suitable. The propellants can be devoid of hydrocarbon propellant gases, which can produce flammable or explosive vapors during spraying. Furthermore, the foams can contain no volatile alcohols, which can produce flammable or explosive vapors during use.

Buffers can be used to control pH of a composition. The buffers can buffer the composition from a pH of about 4 to a pH of about 7.5, from a pH of about 4 to a pH of about 7, or from a pH of about 5 to a pH of about 7. In some embodiments, the buffer can be triethanolamine.

Preservatives can be included to prevent the growth of fungi and microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

In certain embodiments, the formulations can be provided via continuous delivery of one or more formulations to a patient in need thereof. For topical applications, repeated application can be done or a patch can be used to provide continuous administration of the noscapine analogs over an extended period of time.

### Enteral Formulations

The disclosed DNA-based nanostructures can be prepared in enteral formulations, such as for oral administration. Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations containing a disclosed DNA-based nanostructures can be prepared using pharmaceutically acceptable carriers. As generally used herein “carrier” includes, but is not limited to, diluents, preservatives, binders, lubricants, disintegrators, swelling agents, fillers, stabilizers, and combinations thereof. Polymers used in the dosage form include, but are not limited to, suitable hydrophobic or hydrophilic polymers and suitable pH dependent or independent polymers. Suitable hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins. “Carrier” also includes all components of the coating composition which can include plasticizers, pigments, colorants, stabilizing agents, and glidants.

Formulations containing a disclosed DNA-based nanostructures can be prepared using one or more pharmaceutically acceptable excipients, including diluents, preservatives, binders, lubricants, disintegrators, swelling agents, fillers, stabilizers, and combinations thereof.

Delayed release dosage formulations containing a disclosed DNA-based nanostructures can be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

The formulations containing a disclosed DNA-based nanostructures can be coated with a suitable coating material, for example, to delay release once the particles have passed through the acidic environment of the stomach. Suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings can be formed with a different ratio of water soluble polymer, water insoluble polymers and/or pH dependent polymers, with or without water insoluble/water soluble non polymeric excipient, to produce the desired release profile. The coating can be performed on a dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Additionally, the coating material can contain conventional carriers such as plasticizers, pigments, colorants, glidants, stabilization agents, pore formers and surfactants. Optional pharmaceutically acceptable excipients include, but are not limited to, diluents, binders, lubricants, disintegrants, colorants, stabilizers, and surfactants.

Diluents, also referred to as “fillers,” can be used to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful.

Binders can impart cohesive qualities to a solid dosage formulation, and thus can ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders.

Lubricants can be included to facilitate tablet manufacture. Suitable lubricants include, but are not limited to, magnesium stearate, calcium stearate, stearic acid, glycerol behenate, polyethylene glycol, talc, and mineral oil. A lubricant can be included in a tablet formulation to prevent the tablet and punches from sticking in the die. The lubricant can be chosen from such slippery solids as talc, magnesium and calcium stearate, stearic acid and hydrogenated vegetable oils.

Disintegrants can be used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers can be used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

### Additional Active Agents

In some embodiments, an amount of one or more additional active agents are included in the pharmaceutical formulation containing a disclosed DNA-based nanostructures. Suitable additional active agents include, but are not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatoireanti-histamines, anti-infectives, and chemotherapeutics (anti-cancer drugs). Other suitable additional active agents include, sensitizers (such as radiosensitizers). The disclosed DNA-based nanostructures can be used as a monotherapy or in combination with other active agents for treatment or prevention of a disease or disorder.

Suitable hormones include, but are not limited to, amino-acid derived hormones (e.g. melatonin and thyroxine), small peptide hormones and protein hormones (e.g. thyrotropin-releasing hormone, vasopressin, insulin, growth hormone, luteinizing hormone, follicle-stimulating hormone, and thyroid-stimulating hormone), eiconsanoids (e.g. arachidonic acid, lipoxins, and prostaglandins), and steroid hormones (e.g. estradiol, testosterone, tetrahydro testosterone cortisol).

Suitable immunomodulators include, but are not limited to, prednisone, azathioprine, 6-MP, cyclosporine, tacrolimus, methotrexate, interleukins (e.g. IL-2, IL-7, and IL-12), cytokines (e.g. interferons (e.g. IFN-α, IFN-β, IFN-ε, IFN-κ, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g. ibuprofen, naproxen, ketoprofen, and nimesulide), aspirin and related salicylates (e.g. choline salicylate, magnesium salicylae, and sodium salicaylate), paracetamol/acetaminophen, metamizole, nabumetone, phenazone, and quinine.

Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotenergic antidepressants (e.g. selective serotonin reuptake inhibitors, tricyclic antidepresents, and monoamine oxidase inhibitors), mebicar, afobazole, selank, bromantane, emoxypine, azapirones, barbituates, hyxdroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzaprine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, non-steroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylae, and sodium salicaylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene.

Suitable anti-inflammatoires include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, H₁-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H₂-receptor antagonists (e.g. cimetidine, famotidine, lafutidine, nizatidine, rafitidine, and roxatidine), tritoqualine, catechin, cromoglicate, nedocromil, and β2-adrenergic agonists.

Suitable anti-infectives include, but are not limited to, amebicides (e.g. nitazoxanide, paromomycin, metronidazole, tnidazole, chloroquine, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, miltefosine, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethanmbutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpiviirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erthromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, beta lactam antibiotics (benzathine penicillin (benzatihine and benzylpenicillin), phenoxymethylpenicillin, cloxacillin, flucoxacillin, methicillin, temocillin, mecillinam, azlocillin, mezlocillin, piperacillin, amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, nafcillin, cefazolin, cephalexin, cephalosporin C, cephalothin, cefaclor, cefamandole, cefuroxime, cefotetan, cefoxitin, cefiximine, cefotaxime, cefpodoxime, ceftazidime, ceftriaxone, cefepime, cefpirome, ceftaroline, biapenem, doripenem, ertapenem, faropenem, imipenem, meropenem, panipenem, razupenem, tebipenem, thienamycin, azrewonam, tigemonam, nocardicin A, taboxinine, and beta-lactam), quinolones (e.g. lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, aspargainase erwinia chyrsanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, all-trans retinoic acid, and other anti-cancer agents listed elsewhere herein.

DNA Branched Origami Nanostructures, Complexed or Not with LL37 Chimeric Polypeptides

New class of nucleic acid-based self-assembly nanoparticles is disclosed, DNA origami layered with or encapsulated by DNA branches precisely positioned in predetermined locations around DNA origami NP. While previous attempts focused on utilizing unique properties of densely packed nucleic acids on nanoparticle surface, in the form of spherical nucleic acids, DNA dendrimers, or nucleic acid brushes, none were able to at the same time rely on: exclusively self-assembly or have a control of precise nanoparticle shape, or have precise nucleic acid branch positioning in 3D space. We introduce the method to achieve this. We have further developed peptide-based electrostatic attachment strategies, particularly exploring LL37 peptide sequence to impart various functions to such nanoparticles, such as protection of the CpG adjuvant cargo from degradation and functional peptide antigen delivery. In this process, we have made several unexpected discoveries pertaining to these nanoparticles, such as their unique solubility in combination with electrostatic peptides, improved delivery, and unprecedented T cell killing induction. Furthermore, we show a high-density protein antigen coating on the surface of our developed nanoparticles, for the first time approaching this level of payload density, and a very uniform antibody attachment.

An important feature that we have demonstrated as compared to other, particularly solid nucleic acid nanoparticles such as DNA origami, is that the branched nucleic acid surface was able to impart cytosolic delivery properties of its cargo. While small molecules are inefficiently taken up by cells, and nanoparticles enhance their delivery, the key challenge of majority of nanoparticles is that they prevent cytosolic escape of their cargo, but also negatively affect the functions of lysosomes and the autophagy process. Our formulation induced abundant cytosolic signal along with lysosomal colocalization, indicting both, endolysosomal and/or phagolysosomal route as well as cytosolic delivery. As such our method of branch incorporation into nucleic acid NP forms, combined with its targeting abilities, and in combination or not with lipids/fatty acids and/or LL37-peptides or LL37-penetrating peptides is emerging as an ideal novel platform to develop delivery of gene regulating therapy to manage protein expression (e.g., CRISPR Cas9, ASO, siRNA, and others).

DNA Branch Nanostructures, Complexed with LL37 Chimeric Polypeptides, and Encapsulated or Not in Alginate

Formulations based on electrostatic attachment are difficult to translate to in vivo I.V. route delivery due to their aggregation and instability. Surprisingly, our LL37-SIINFEKL coated CpG/Branches were able to induce a significant level of T cell killing in vivo via I.V. route, which is for the first time demonstrated for nucleic acid NP and electrostatic peptide-based attachment by our formulation. Most importantly, a very surprising ability of LL37 electrostatic attachment sequence to remain stable in alginate controlled release delivery system was discovered, leading to unprecedented intratumoral killing activity and prolonged survival in B16-F10 mouse melanoma model.

Methods of Using DNA Branches and DNA Branched Origami Nanostructures, Complexed or Not with LL37 Chimeric Polypeptides, and Encapsulated or Not in Alginate

The disclosed DNA-based nanostructures can be used to deliver one or more cargo compounds to a subject in need thereof or a cell. In some embodiments, the disclosed DNA-based nanostructures can be used to deliver an RNA or DNA molecule for replacement gene/transcript therapy, deliver RNAi or similar RNA (e.g. microRNA) to a subject to specifically inhibit RNA transcripts to reduce gene expression of a specific gene or genes, deliver an imaging agent, delivering a small molecule drug, and/or deliver any other cargo compound that can be loaded in the disclosed DNA-based nanostructures. Thus, the disclosed DNA-based nanostructures can be used to deliver a treatment, prevention, and/or a diagnostic compound to a subject in need thereof.

The disclosed DNA-based nanostructures can be used in some cases to treat a subject with a cancer. The cancer of the disclosed methods can be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

In some embodiments, where the disclosed DNA-based nanostructures include a photocleavable linker that is linking the targeting moiety and/or cargo compound the DNA-based nanostructures can be administered to the subject or population of cells. After administration, light can be applied to the region and/or population of cells in the subject in need thereof where treatment or prevention is needed to cause the release of the disclosed DNA-based nanostructures and/or cargo molecule.

The disclosed DNA-based nanostructures as provided herein can be administered to a subject in need thereof, cell, or population thereof. The subject in need thereof can have a cancer, genetic disease or disorder, a viral, bacterial, parasitic, and/or fungal infection, or any other disease or disorder that would benefit from an effective agent (such as a cargo compound described herein) being delivered. The amount delivered can be an effective amount of a DNA-based nanostructures provided herein. The subject in need thereof can be symptomatic or asymptomatic. In some embodiments, the DNA-based nanostructures provided herein can be co-administered with another active agent. It will be appreciated that co-administered can refer to an additional compound that is included in the formulation or provided in a dosage form separate from the DNA-based nanostructures or formulation thereof. The effective amount of the DNA-based nanostructures or formulation thereof, such as those described herein, can range from about 0.1 mg/kg to about 500 mg/kg. In some embodiments, the effective amount ranges from about 0.1 mg/kg to 10 mg/kg. In additional embodiments, the effective amount ranges from about 100 mg/kg. If further embodiments, the effective amount ranges from about 0.1 mg to about 1000 mg. In some embodiments, the effective amount can be about 500 mg to about 1000 mg.

Administration of the DNA-based nanostructures and formulations thereof can be systemic or localized. The compounds and formulations described herein can be administered to the subject in need thereof one or more times per day. In an embodiment, the compound(s) and/or formulation(s) thereof can be administered once daily. In some embodiments, the compound(s) and/or formulation(s) thereof can be administered given once daily. In another embodiment, the compound(s) and/or formulation(s) thereof can be administered is administered twice daily. In some embodiments, when administered, an effective amount of the compounds and/or formulations are administered to the subject in need thereof. The compound(s) and/or formulation(s) thereof can be administered one or more times per week. In some embodiments the compound(s) and/or formulation(s) thereof can be administered 1 day per week. In other embodiments, the compound(s) and/or formulation(s) thereof can be administered 2 to 7 days per week.

In some embodiments, the DNA-based nanostructures(s) and/or formulation(s) thereof, can be administered in a dosage form. The amount or effective amount of the compound(s) and/or formulation(s) thereof can be divided into multiple dosage forms. For example, the effective amount can be split into two dosage forms and the one dosage forms can be administered, for example, in the morning, and the second dosage form can be administered in the evening. Although the effective amount is given over two doses, in one day, the subject receives the effective amount. In some embodiments the effective amount is about 0.1 to about 1000 mg per day. The effective amount in a dosage form can range from about 0.1 mg/kg to about 1000 mg/kg. The dosage form can be formulated for oral, vaginal, intravenous, transdermal, subcutaneous, intraperitoneal, or intramuscular administration. Preparation of dosage forms for various administration routes are described elsewhere herein.

### EXAMPLE EMBODIMENTS

### Embodiment 1

A nanoparticle, comprising a base nanostructure, made out of non-branching and non-dendrimer nucleic acid nanostructure formed from a single or plurality of nucleic acid scaffold strands and a single or plurality of nucleic acid staple strands assembled into a geometry (e.g. DNA origami nanoparticle), wherein this base nucleic acid nanostructure comprises one or more first single stranded nucleic acid oligonucleotide attachment arms configured to directly bind to a first complementary nucleic acid oligonucleotide strands further comprising a branched oligonucleotide dendrimer comprising a plurality of second single stranded nucleic acid oligonucleotide attachment arms and at least one first complementary nucleic acid oligonucleotide strand, wherein the second single stranded nucleic acid oligonucleotide attachment arms are configured to bind to second complementary nucleic acid oligonucleotide strands, wherein the branched oligonucleotide dendrimer is assembled with double stranded nucleic acids and optionally single stranded regions and is made exclusively out of nucleic acids through self-assembly using the principle of nucleic acid oligonucleotide complementarity and non-complementarity, wherein the overall nucleic acid nanostructure comprises a peptide sequence of interest (e.g., antigen peptide, cell penetrating peptide, etc), which is synthesized to contain additional peptide sequence/s that allow attachment of the peptide sequence of interest to the nucleic acid polymers of the nanostructure by electrostatic interaction.

### Embodiment 2

A nanoparticle exclusively made of nucleic acids and ions, comprising a base nanostructure, made out of non-branching and non-dendrimer nucleic acid nanostructure formed from a single or plurality of nucleic acid scaffold strands and a single or plurality of nucleic acid staple strands assembled into a geometry (e.g. DNA origami nanoparticle), wherein this base nucleic acid nanostructure comprises one or more first single stranded nucleic acid oligonucleotide attachment arms configured to directly bind to a first complementary nucleic acid oligonucleotide strands further comprising a branched oligonucleotide dendrimer comprising a plurality of second single stranded nucleic acid oligonucleotide attachment arms and at least one first complementary nucleic acid oligonucleotide strand, wherein the second single stranded nucleic acid oligonucleotide attachment arms are configured to bind to second complementary nucleic acid oligonucleotide strands, wherein the branched oligonucleotide dendrimer is assembled with double stranded nucleic acids and optionally single stranded regions and is made exclusively out of nucleic acids through self-assembly using the principle of nucleic acid oligonucleotide complementarity and non-complementarity.

### Embodiment 3

The nanoparticle of embodiment 2, wherein the base nanostructure is completely or partially assembled using a DNA origami technique (or nucleic acid origami technique), or other nucleic acid self-assembly techniques to direct and organize the origin of nucleic acid-made branches on or within the base nanostructure using exclusively nucleic acids material and the 3D nature of the base nanostructure and branches themselves, while nucleic acid-based branches further control the topography and density of the structure, including based on the length of branching units and the branching density or frequency, including the topography, density, and spacing of attachment sites for various functions and various molecules of interest, and optionally further complexed with peptides based on electrostatic attachment or attachment with nucleic acid binding molecules such as LL37 peptide, anthracyclines, etc.

### Embodiment 4

The nanoparticle of embodiment 2, comprising one or more peptide sequences of interest (e.g., antigen peptides, cell penetrating peptides, ligand targeted peptides, polypeptides such as proteins, etc), which is extended via peptide bonds (i.e., amide bonds) synthesis to contain additional peptide sequence/s that allow attachment of the peptide sequences of interest to the nucleic acid polymers of the nanoparticle of claim 2 by electrostatic or other interactions.

### Embodiment 5

A method of using the embodiment of claim 2, wherein the nucleic acid branches on nucleic acid base nanoparticle: a) enhance the surface area of nanoparticle, molecular weight, geometric size, and capacity and nature of payload attachment of nucleic acid nanoparticles for enhanced functions, such as increased loading of electrostatically bound peptides onto DNA origami base nanoparticle, b) provide unique electrostatic attachment modality with nucleic acid binding peptides through branching arms that collapse to complex with nucleic acid binding peptides, wherein at least two or more distinct and distanced locations by more than 20 bases apart on the nucleic acid nanostructure are electrostatically contacting and binding peptides simultaneously, such as the ability of the two opposite arms of branched nucleic acid structures to interact with peptide simultaneously. c) control molecule spacing of for example CpG, including real-time adaptable (i.e., flexible) spacing to match exact distance needed for receptor dimerization or multivalent binding, where the two or more adjacent single stranded arms have the flexibility to bind to two or more distinct positions on dimer or other receptors with the distance range of zero to fifteen nanometers between the binding spots, d) create cavities and steric hinderance to provide protection of molecules positioned between the nucleic acid base nanoparticle and nucleic acid branches, including electrostatically bound peptides, wherein peptide is shielded from the external biological environment via its incorporation within the branched nucleic acid network, e) modulate and/or enhance the solubility and stability of nucleic acid base nanoparticles, such as DNA origami structures electrostatically complexed with peptides.

### Embodiment 6

A nanoparticle of any one of embodiments 1 to 4, wherein each branched oligonucleotide dendrimer comprises at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 second single stranded nucleic acid oligonucleotide attachment arms.

### Embodiment 7

A branched nucleic acid-made dendrimer nanostructure electrostatically complexed with peptides, polypeptides, or portions of peptides containing amino acids with amine groups, guanidine groups, or positive charges, whereby branched nucleic acid dendrimer nanostructure provides unique electrostatic attachment modality with nucleic acid binding peptides through branching arms that collapse to complex with nucleic acid binding peptides, wherein at least two or more distinct and distanced locations by more than 20 bases apart on the nucleic acid nanostructure are electrostatically contacting and binding peptides simultaneously, such as the ability of the two opposite single- or double-stranded arms of branched nucleic acid structures to interact with peptide simultaneously.

### Embodiment 8

A nanostructure of any one of embodiments 1, 4, and 7, wherein peptide sequences of interest are extended via peptide bond synthesis to contain LL37 peptide sequence (LLGDFFRKSKEKIGKEFKRIVQRIKDFLRNLVPRTES (SEQ ID NO: 1)).

### Embodiment 9

A nanostructure of any one of embodiments 1, 4, 7, and 8, containing nucleic acid CpG adjuvant, or other nucleic acid-based adjuvants.

### Embodiment 10

A nanostructure of any one of embodiments 1, 4, 7, 8, and 9, encapsulated in alginate capsules.

### Embodiment 11

A nanostructure of any one of embodiments 1, 4, 7, 8, 9, and 10, injected intratumorally.

### Embodiment 12

Any nanostructure of embodiment 8, injected intravenously.

### Embodiment 13

Any nanostructure of embodiment 8, injected into the vessel, such as vein, artery, or lymphatic vessel.

### Embodiment 14

The property of LL37 peptide sequence (LLGDFFRKSKEKIGKEFKRIVQRIKDFLRNLVPRTES (SEQ ID NO:1)), or LL37-containing peptide chimera (i.e., comprising additional peptides or polypeptides of interest such as peptide antigens, protein antigens, etc), complexed or not with nucleic acids or preformed nucleic acid structures, as compared to other nucleic acid-binding peptides to: a) be retained within alginate capsule as opposed to premature leakage, to for example, allow controlled release of LL37 and/or LL37 containing forms (e.g., LL37 complexed with nucleic acids or nucleic acid nanostructures), b) to minimize unwanted binding of the chimeric peptide or polypeptide to certain extracellular matrix components such as gelatin, c) to facilitate delivery and functional effects via intravenous route. Embodiment 15. A method for stimulating anti-tumoral effect via intratumoral injection of alginate capsules containing LL37 (LLGDFFRKSKEKIGKEFKRIVQRIKDFLRNLVPRTES (SEQ ID NO:1)) peptide fused or not with an antigen and complexed with nucleic acid CpG adjuvants or nucleic acid nanostructures containing adjuvants.

### Embodiment 16

A method for stimulating anti-tumoral effect via intratumoral injection of LL37 (LLGDFFRKSKEKIGKEFKRIVQRIKDFLRNLVPRTES (SEQ ID NO:1)) peptide fused with an antigen and complexed with nucleic acid CpG adjuvants or nucleic acid nanostructures containing adjuvants.

### Embodiment 17

A peptide delivery system comprising LLGDFFRKSKEKIGKEFKRIVQRIKDFLRNLVPRTE-SSIINFEKL (SEQ ID NO:2) peptide and nucleic acids (e.g., nucleic acid adjuvants and nucleic acid nanostructures), where SIINFEKL (SEQ ID NO:3) portion of the sequence is a model antigen peptide that can be replaced with any peptide sequence or series of peptide sequences, each of which can be unique (e.g., antigen peptides, ligand targeting peptides, polypeptides such as proteins), and where sequences such as KLALRVRRALISLEQLE (SEQ ID NO:4) and TEW can be included in the overall peptide sequence, which have cell penetrating or peptide cleavage or proteasome directing functions can be replaced with different sequences performing similar functions and can be repositioned within the overall peptide sequence.

### Embodiment 18

A method of protecting branched nucleic acid dendrimer nanostructure via electrostatic complexation with peptides.

### Embodiment 19

A method of protecting branched nucleic acid nanostructures via electrostatic complexation with LL37 peptides, or sequences containing LL37 sequence.

### Embodiment 20

A method of mediating cytosolic delivery via electrostatic complexation of nucleic acid nanostructures with LL37 peptides or sequences containing LL37 sequence via peptide bonds.

### Embodiment 21

A method of mediating cytosolic delivery of nucleic acids and their forms (e.g., DNA origami nanoparticles, mRNA) by the surrounding branched nucleic acid structure layer.

### Embodiment 22

The method of embodiment 21, wherein cytosolic delivery is further mediated via electrostatic complexation of forms produced by the method of embodiment 21 with LL37 peptides or sequences containing LL37 sequence made via peptide bonds.

### Embodiment 23

A nanoparticle of any one of embodiments 1 and 4, wherein at least 1,000 peptide antigens are attached to the nucleic acid nanostructure via electrostatic interaction.

### Embodiment 24

A nanoparticle of any one of embodiments 1 and 4, comprising at least 50, 60, 70, 80, 90, 100 nM peptide.

### Embodiment 25

The nanoparticle of embodiment 1, wherein the peptide comprises at least 5, 6, 7, 8, 9, or 10 contiguous positively charged amino acids.

### Embodiment 26

The nanoparticle of embodiment 1, wherein the peptide (e.g., peptide antigen) comprises cell penetrating peptides and/or antimicrobial peptides at the N- and/or C-terminus for tuning the peptide electrostatic attachment to nucleic acid nanostructures, increased cell uptake and cytosolic delivery, and further may contain at least one peptide cleavage site (e.g., cathepsin cleavage site, furin cleavage site, etc.), and/or at least one immunoproteasome processing site for peptide processing containing the following example sequence: LLGDFFRKSKEKIGKEFKRIVQRIKDFLRNLVPRTESKLALRVRRALISLEQLESIINFEKLT EW (SEQ ID NO:5), where SIINFEKL (SEQ ID NO:3) portion of the sequence is a model antigen peptide that can be replaced with any antigen peptide sequence.

### Embodiment 27

The nanoparticle of embodiment 1, wherein the at least 5, 6, 7, 8, 9, or 10 continuous positively charged amino acids are at the N-terminus of the peptide antigen.

### Embodiment 28

The nanoparticle of embodiment 1, wherein the at least 5, 6, 7, 8, 9, or 10 continuous positively charged amino acids are at the C-terminus of the peptide antigen.

### Embodiment 29

The nanoparticle of embodiment 1, wherein the positively charged amino acids are lysine or arginine amino acids.

### Embodiment 30

The nanoparticle of embodiment 1, wherein the peptide antigen comprises at least 10 contiguous lysine amino acids.

### Embodiment 31

The nanoparticle of embodiment 1, wherein the peptide antigen comprises at least 10 contiguous arginine amino acids.

### Embodiment 32

The nanoparticle of embodiment 1, comprising at least 30, 35, 40, 45, 50, 60, 70, 80, 90, 100 peptides per nm².

### Embodiment 33

The nanoparticle of embodiment 1, wherein each of the plurality of scaffold strands are 300 to 15,000 nucleotides in length,

### Embodiment 34

The formulation pertaining to any one of embodiments 1 to 33, wherein the peptide antigen comprises a viral antigen.

### Embodiment 35

The formulation pertaining to any one of embodiments 1 to 33, wherein the peptide antigen comprises a tumor specific neoantigen and/or tumor associated antigen.

### Embodiment 36

The formulation pertaining to any one of embodiments 1 to 35, wherein the nucleic acid nanostructure comprises one or more first single stranded nucleic acid oligonucleotide attachment arms configured to bind to a first complementary nucleic acid oligonucleotide strands.

### Embodiment 37

The formulation pertaining to any one of embodiments 1 to 36, further comprising a plurality of nucleic acid adjuvant molecules conjugated to first complementary nucleic acid oligonucleotide strands or second complementary nucleic acid oligonucleotide strands.

### Embodiment 38

The formulation of embodiment 37, wherein the plurality of nucleic acid adjuvant molecules are CpG molecules.

### Embodiment 39

A nanoparticle of any one of embodiments 1 and 2, wherein the base nucleic acid nanostructure comprises a single or multiple cavities, wherein the one or more first single stranded oligonucleotide attachment arms containing branched nucleic acid dendrimers are positioned inside and/or outside of the cavity, or within the base nucleic acid nanostructure.

### Embodiment 40

A nanoparticle of any one of embodiments 1 and 2, further comprising one or more targeting ligands conjugated to the first complementary nucleic acid oligonucleotide strands or second complementary nucleic acid oligonucleotide strands.

### Embodiment 41

A method for vaccinating a subject, comprising administering to the subject the vaccine device of any one of embodiments 1 to 40.

### Embodiment 42

A non-DNA origami nucleic acid nanostructure formed from a nucleic acid material and optionally therapeutic, targeting, sensing, imaging, detection, and building materials (e.g., phospholipids), comprising a peptide of interest, which is extended via peptide bond (i.e., amide bond) synthesis to contain additional peptide sequence/s that allow attachment of the peptide sequences of interest to the nucleic acid polymers of the nanoparticle by electrostatic or other interactions.

### Embodiment 43

The nanoparticle of embodiment 42, wherein the peptide sequence of interest can be polypeptide or protein.

### Embodiment 44

A nucleic acid dendrimer nanostructure that is made by self-assembly of unique predefined sequences at predefined molar ratios in one step (i.e., one pot synthesis) in the presence of only 1× phosphate buffered saline and require no purification procedures after the self-assembly process.

### Embodiment 45

A nucleic acid dendrimer nanostructure that is made by self-assembly of unique predefined sequences that do not require oligonucleotide purification such as HPLC or PAGE before self-assembly process and are assembled at predefined molar ratios in one step (i.e., one pot synthesis) in the presence of only 1× phosphate buffered saline and require no purification procedures after the self-assembly process.

### Embodiment 46

A nucleic acid nanovaccine delivery platform that can directly incorporate FDA approved oligonucleotide adjuvants (e.g., CpG) by direct attachment to the nucleic acid structure, without the need for modification of oligonucleotide adjuvant, while preserving adjuvant function and potency.

### Embodiment 47

The nanoparticle of embodiment 1, wherein the peptide comprises 1 to 5 contiguous positively charged amino acids.

### Embodiment 48

The nanoparticle of embodiment 1, wherein the peptide comprises 1 to 3 contiguous positively charged amino acids.

### Embodiment 49

A nucleic acid structure, comprising a nucleic acid origami nanostructure formed from a plurality of scaffold strands and a plurality of staple strands assembled into a geometry, wherein the nucleic acid nanostructure comprises one or more first single stranded oligonucleotide attachment arms configured to bind to a first complementary oligonucleotide strands, further comprising a branched oligonucleotide dendrimer comprising a plurality of second single stranded oligonucleotide attachment arms and at least one first complementary oligonucleotide strand, wherein the second single stranded oligonucleotide attachment arms are configured to bind to second complementary oligonucleotide strands.

A number of embodiments of the invention have been described. Nevertheless, it will be understood that various modifications may be made without departing from the spirit and scope of the invention. Accordingly, other embodiments are within the scope of the following claims.

### EXAMPLES

The following examples describe DNA branched origami-based devices, DNA branches, LL37-based chimeric peptides, and alginate, as well as their methods of use, such as enhancing or better controlling density and positioning of molecules in 3D space around DNA origami, enhancing therapeutic index (i.e., therapeutic efficacy in relation to side effects) of vaccine and other cargoes. Potential uses span many delivery and sensing applications incorporating described DNA nanoparticles. Examples also describe or demonstrate discovery of novel properties of LL37-based chimeric peptides, their unique interactions with DNA branches or DNA branched origami nanostructures, as well as the discovery of unique interactions and properties of LL37-based chimeric peptides with alginate, and unexpected drastic enhancement of anti-tumor efficacy for alginate-encapsulated LL37gp100 complexed CpG-loaded DNA branches. Several other unexpected discoveries are demonstrated throughout examples described below.

### Example 1

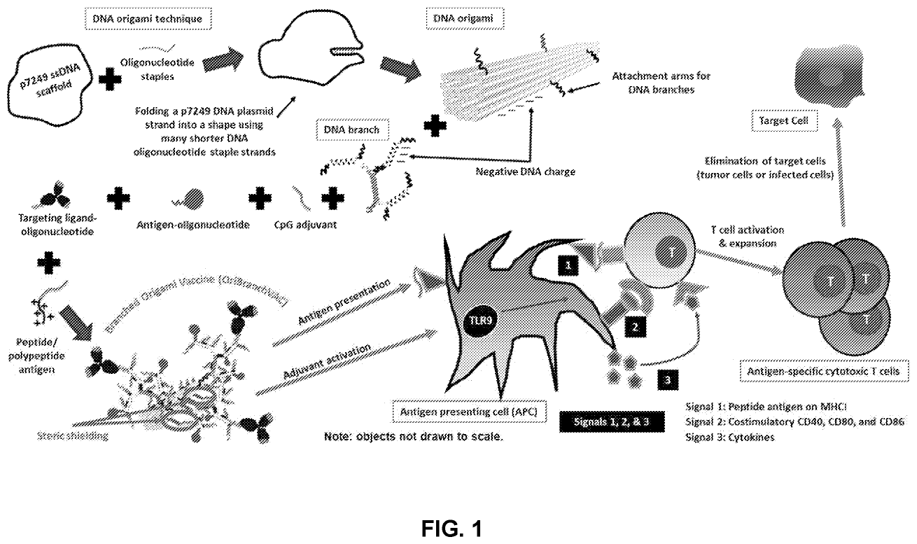

Example 1, illustrated by FIG. 1, shows a workflow towards optimizing a DNA branched origami vaccine (OriBranchVAC). An embodiment of the disclosed branched origami vaccine contains a DNA origami nanoparticle which represents a core nucleic acid nanoparticle construct containing free single-stranded oligonucleotide attachment arms for the attachment of nucleic acid branches (e.g., DNA branches). In this manner, nucleic acid branches are precisely positioned in 3D space around the core nucleic acid nanoparticle at a predetermined density, where nucleic acid branches have free arms for attaching CpG adjuvants, other nucleotide-based drugs, or any molecule that can be directly conjugated to these arms or conjugated to oligonucleotide arms complementary to the attachment arms of branches. Alternatively, some free arms on the core nanoparticle can be used to attach molecules to provide shielding from biological environment, such that these molecules are hidden in between the surface of the core nucleic acid nanoparticle and the coat of nucleic acid branches, where nucleic acid branches provide protection to the underlying molecule by steric hinderance and bio-physicochemical properties. Another alternative is that a molecule is incorporated within the core nucleic acid nanoparticle, such as for example DNA-binding molecule, where the layer of nucleic acid branches then serves as a sponge to sequester a DNA-binding molecule, therefore reducing leakage. Various DNA branched origami vaccine formulations were designed, using different number of branching units, and electrostatic-based polypeptide attachment methods. Peptide antigens can be assembled using chimeric polypeptides via electrostatic attachment, containing antigenic peptide region and a nucleic acid binding region, such as LL37 peptide sequence. These formulations were evaluated for cargo attachment and stability, size and geometry, cell binding, uptake, and trafficking, biodistribution, activation of antigen presenting cells and antigen delivery, T cell cytotoxic function and anti-tumor efficacy. These delivery methods may be useful in treatments against viruses, bacteria, and cancers, among many other applications.

### Example 2

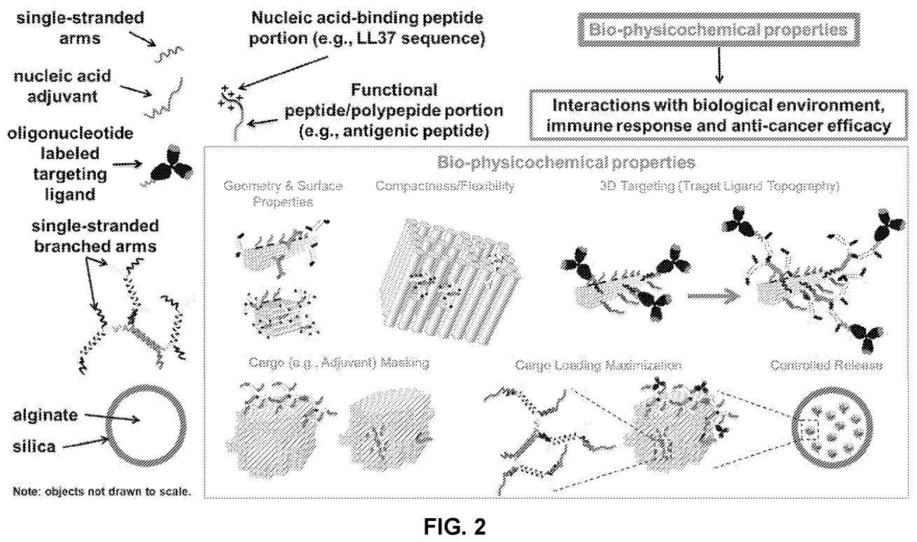

Example 2, illustrated by FIG. 2, shows a schematic pertaining to an embodiment of unique bio-physicochemical properties of branched origami vaccine formulations. The core nucleic acid nanoparticle can have a rigid or soft geometry (i.e., size and shape) of any kind, determining a specific shape for nucleic acid branch coat, while the length between each branching point, the number of units and branching points in each branch, and the surface density and chemical character of branches control the surface character. Furthermore, branched nucleic acids can be incorporated throughout the core nucleic acid nanoparticle, to modulate the compactness and flexibility of the core nanostructure. Branches provide a unique control of 3D topography on the core nucleic acid nanoparticle for various molecules such as targeting ligands and antigens, while providing flexibility and freedom of molecule motion as compared to non-branched nucleic acid structures; unique method to modulate ligand-targeting parameters via branched origami design to increase the number and flexibility of presentation of targeting ligands is shown. Nucleic acid branches can be attached to the core nucleic acid nanostructure on the surface or inside the cavities of the core nanoparticle, and maximize the loading capacity of the core nanostructure. Shown is incorporation of adjuvants into origami nanoparticle cavity to improve masking of adjuvants and reduce their release in circulation, which may improve vaccine safety. Next, shown is a concept design of branch assembly in the internal cavity to increase the attachment density inside the core nucleic acid nanoparticle. Lastly, alginate or other polymers can be used to encapsulate branched origami therapeutic for extended release and protection against degradation.

### Example 3

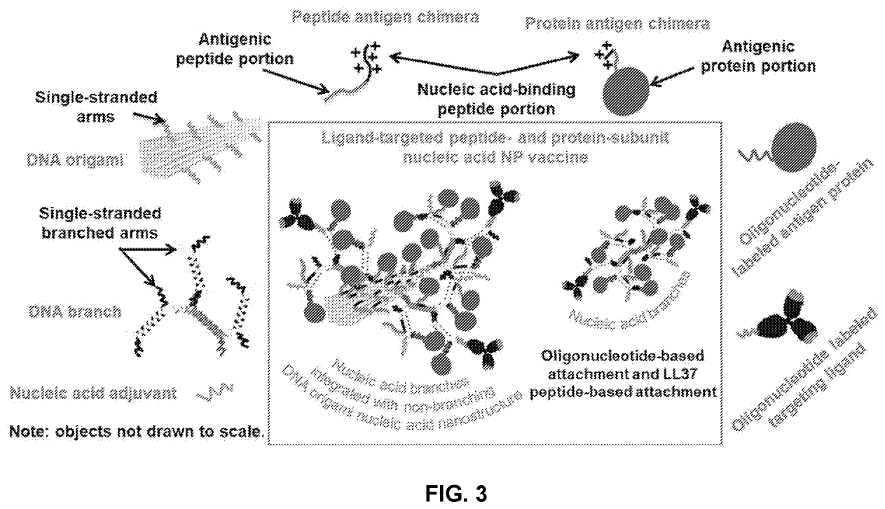

Example 3, illustrated by FIG. 3, shows a schematic pertaining to an embodiment of chimeric LL37-polypeptide coated branch or branched origami subunit vaccine for combined peptide and protein antigen cargo loading. Presented are branch and branched origami delivery platforms utilizing dual antigen attachment technology consisting of oligonucleotide-based and electrostatic-based peptide and protein antigen attachment for strong induction of both, humoral and cellular immune responses. Branched design allows for high adjuvant, antigen, and ligand targeting attachment capacity simultaneously. The branched origami design has a precise and predetermined branch structure with terminal attachment arms whose number, density, and geometry are precisely defined, which can allow for targeting ligands to multiple target epitopes and to multiple cell types simultaneously (e.g., dendritic cells, macrophages, B cells, T cells, cancer cells, etc.) on a single branched origami nanostructure without compromising on the sufficient number of attached ligands or molecules; this is particularly useful for the ligands of relatively low affinity such as aptamers, or to direct one type of cell towards the other type of cell for various purposes. In addition, combination targeting to different target epitopes on the same cell type, as well as targeting multiple cell types is a known strategy to reduce off-site targeting; branched origami design may improve this strategy. Lastly, high coating of nucleic acid nanoparticles, particularly with proteins such as antibodies has been shown to protect DNA nanoparticle half-life in serum and in the body.

Described example allows for potentially unlimited branching of origami attachment arms for ultrahigh payload capacity loading. This maximizes surface density of attachment sites beyond any current existing DNA origami methods or designs. In general, the number of oligonucleotide-based surface attachment sites on DNA origami is limited due to several aspects of DNA origami pertaining to the need to balance the number of attachment sites with: 1) the cost of the staple strands by keeping the strands as short as possible, and also 2) inherent disadvantage of introduction of mistakes as the synthesized strands get longer, 3) the stability of DNA origami or attachment arms itself, as higher number of attachment arms lead to shorter portions of these attachment oligonucleotide arms acting as staple segments in DNA origami, 4) potential aggregation problems with high attachment arm numbers. In addition, current methods do not offer attachment of molecules further away from the origami surface (for example 50 or 100 nm away from DO surface).

Although DNA branching has been previously utilized to make DNA nanoparticles, it has never been used to increase the density of attachment spots on DNA origami. While DNA branched nanoparticles with high attachment density lack the ability of precise geometrical control and lack defined surface, DNA origami offers these advantages, but lacks high attachment sites. The utilization of a DNA branched origami design is a new application of nucleic acid branches and DNA origami technique, where DNA origami is utilized to start and determine the starting surface topography and curvature (i.e., surface organization) of where branches start to propagate. This is in contrast to the standalone branches which are starting to branch from a very small point in space, which does not allow the maximum density of branching in the core due to the starting high surface curvature of such a small space, a phenomenon well known for a very small nanoparticles (e.g. 1-10 nm).

### Example 4

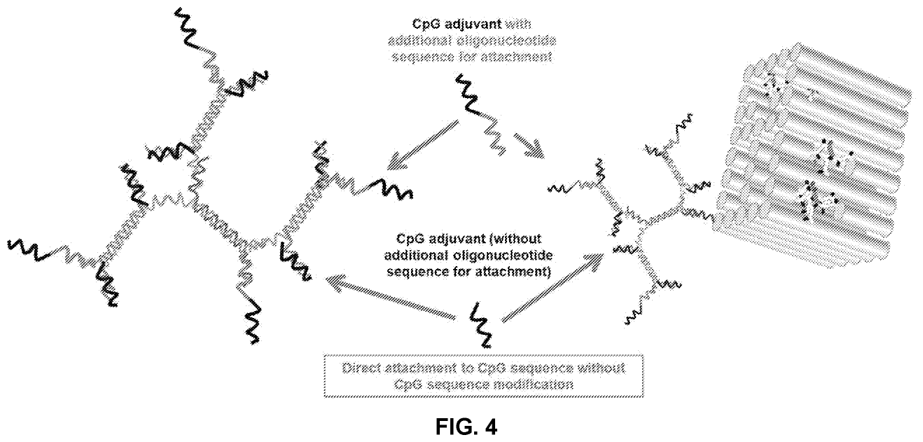

Example 4, illustrated by FIG. 4, shows a schematic pertaining to an embodiment of direct CpG attachment to a nucleic acid nanostructure, where the free attachment arm sequence on the nucleic acid nanostructure is complementary to the full length of one of the commercially available or FDA approved nucleic acid sequences (e.g., CpG1826, etc), i.e., forming a double strand along the full length of CpG, which may give new properties to nucleic acid adjuvants. Other designs can utilize branches where branches themselves incorporate CpG sequences or other therapeutic sequences needed for particular applications, but efficiency and uniformity of such designs may be reduced.

### Example 5

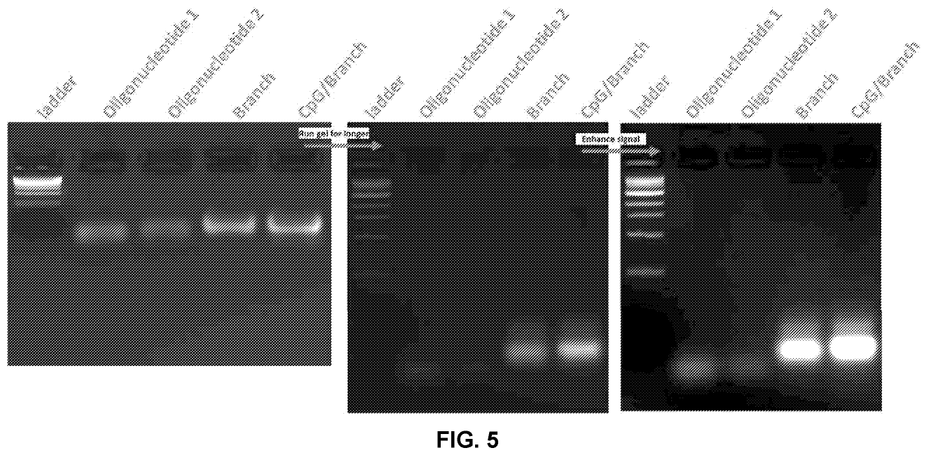

Example 5, illustrated by FIG. 5, shows assembly of an embodiment of DNA branches in PBS and a direct CpG attachment to nucleic acid branch using agarose gel electrophoresis. Note that there was no shift in bands after CpG attachment due to the direct CpG attachment method, where the full 20 bases of CpG1826 are double stranded with DNA branch arms. Tight bands indicate precise and uniform product, while the lack of oligonucleotide bands under the band corresponding to DNA branch nanoparticles indicate complete annealing of oligonucleotides into a DNA branch, requiring no further purification steps. The unique branch sequences allow for one pot synthesis where all the components can be added at once in appropriate molar ratios.

### Example 6

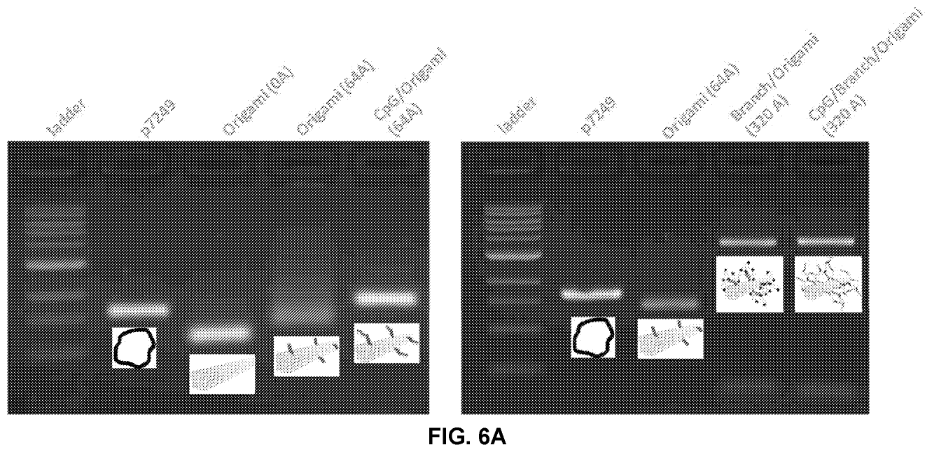

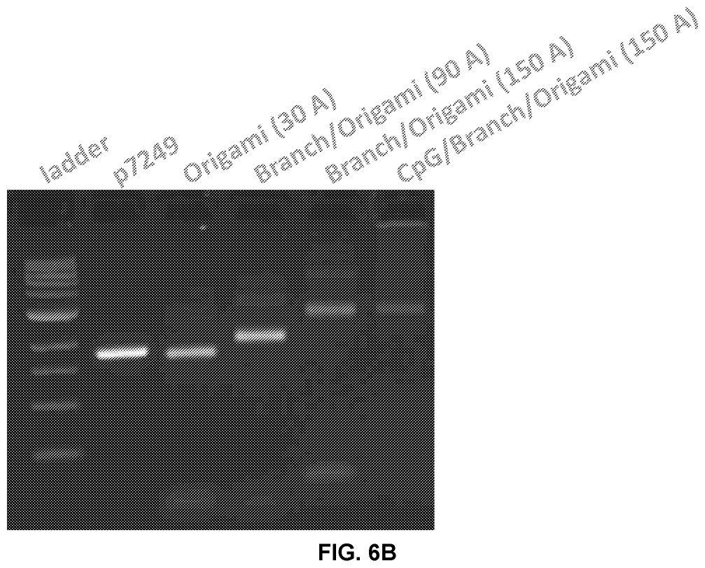

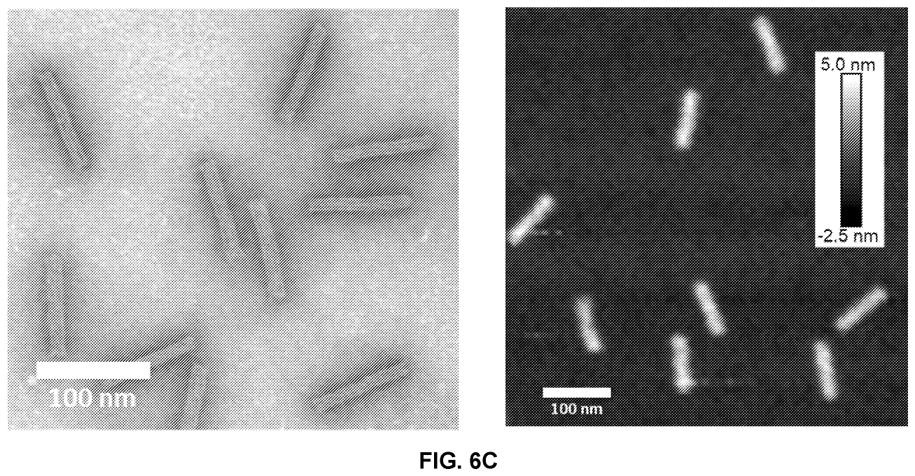

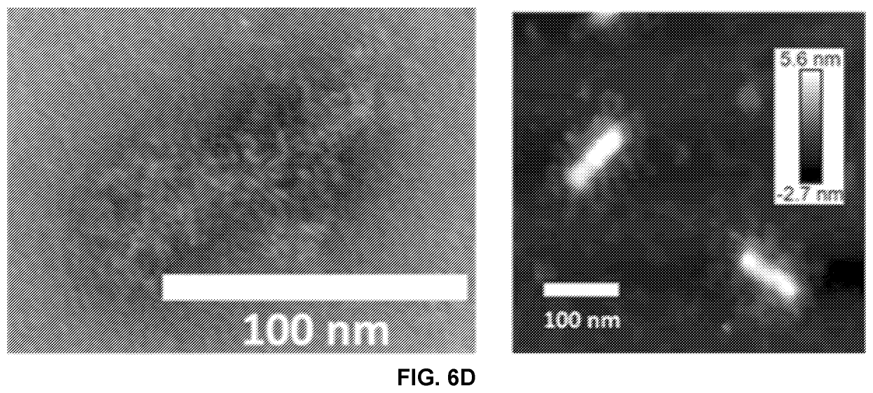

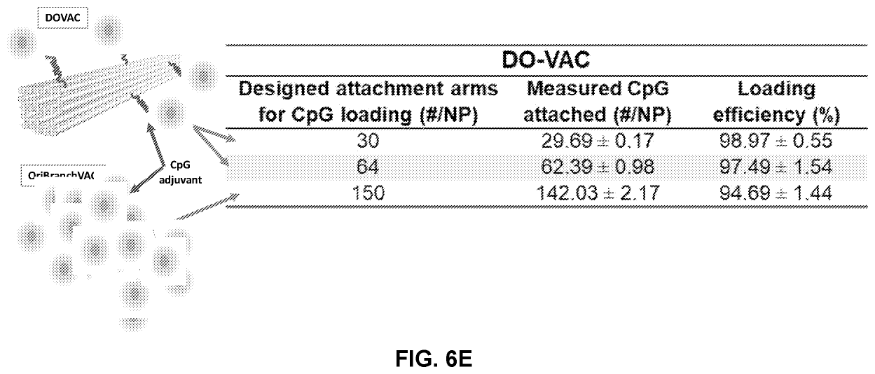

Example 6, illustrated by FIGS. 6A to 6E, shows precise and efficient self-assembly of an embodiment of a high payload capacity DNA branched origami with various numbers of branched oligonucleotide arms for attachment of diverse cargoes of interest and with high cargo attachment efficiency. Tight bands indicate precise and uniform products. FIG. 6A shows agarose electrophoresis gels, that based on the band shifts at each step of synthesis, indicate successful manufacturing of DNA origami and CpG attachment on the left, while precise equimolar self-assembly of DNA branches on DNA origami and subsequent attachment of CpG is shown on the right. Again, note on the right that there is no shift in bands after CpG attachment to branched origami due to the direct CpG attachment method, where the full 20 bases of CpG1826 are double stranded with DNA branch arms. In comparison, it can be seen on the left that there is a shift when CpG is attached using a regular method where CpG is designed with a complementary oligonucleotide extension for attachment to DNA origami attachment arms. FIG. 6B shows a different number of starting attachment arms on DNA origami, subsequent precise equimolar self-assembly of DNA branches with different number of branching units and attachment arms, and finally, the direct attachment of CpG. DNA origami with 30 and 64 attachment arms utilize common technique of designing attachment arms or overhangs in DNA origami field. Branched origami with 90, 150, and 320 branched attachment arms are examples of addition of DNA oligonucleotides designed using specific design rules to result in complete and efficient self-assembly of DNA branches on DNA origami. These rules include limiting self-binding sequence regions, limiting non target oligonucleotide binding, etc. FIG. 6C shows transmission electron microscopy (TEM, left) and atomic force microscopy (AFM, right) of DNA origami with 64 regular attachment arms showing uniform geometry, but regular attachment arms were too small to visualize as expected. FIG. 6D shows DNA branching layer encapsulating/layering DNA origami, where branches and/or branched attachment arms were clearly visible (TEM, left; AFM, right). The flexibility of branches and their collapse on origami surface was indicated by some of the branches being bent or not visible during drying process, often requiring AFM imaging in liquid conditions. FIG. 6E shows quantification of attachment of model molecule (fluorophore labeled CpG adjuvant) to regular or branched design attachment arms, demonstrating high attachment efficiency of over 94% and increasing payload capacity for increasing number of arm attachment sites. Fluorescence intensity, standard curve, and DNA optical density measurements were used to determine CpG concentration and DNA nanoparticle concentration for each sample after purification to determine the number of molecules attached per single NP.
Currently, DNA origami is limited in the number of attachment arms to less than 150 attachment arms per NP, and most designs have around 40 to 100 attachment arms. Furthermore, the topography of surface arms on DNA origami is limited as well, and is determined primarily by the DNA origami geometry and constrained to a couple of tenths of nm from the DNA origami surface. The disclosed method may be useful to allow for controlled topography away from DNA origami surface, and utilization of 3D space on DNA origami surface. In addition, DNA branching on DNA origami can presumably be used for modification of DNA origami nanoparticle surface flexibility as well as modulation of freedom of movement and thermal energy of attached ligands by controlling the length and the degree of branching of DNA branches attached to DNA origami. Surface character determines interactions with biological environment, for example protein adsorption, and even biofouling. This DNA branching method also allows for consequent growth in diameter and MW of DNA origami nanoparticles, one of the current limitations of DNA origami technique due to limited scaffold sizes. Potential applications are numerous, from sensing, imaging, and even reducing the leakage of intercalating drugs from DNA origami in circulation and in the body. The density of branched arms can be designed so high, that the layer of arms may be able to act as a sponge to slow down and control the release of chemotherapy drugs and other drugs. For this method, DNA origami can be first loaded with intercalating agents, and subsequently, high density DNA branches can then be self-assembled onto DNA origami to provide a barrier layer with high affinity for intercalating agent such as that upon the leakage of intercalating agent from DNA origami, intercalating agent is caught and intercalated by the network of DNA branches on the surface. This has application in for example reducing cardiotoxicity from chemotherapy based on intercalating agents such as doxorubicin.

### Example 7

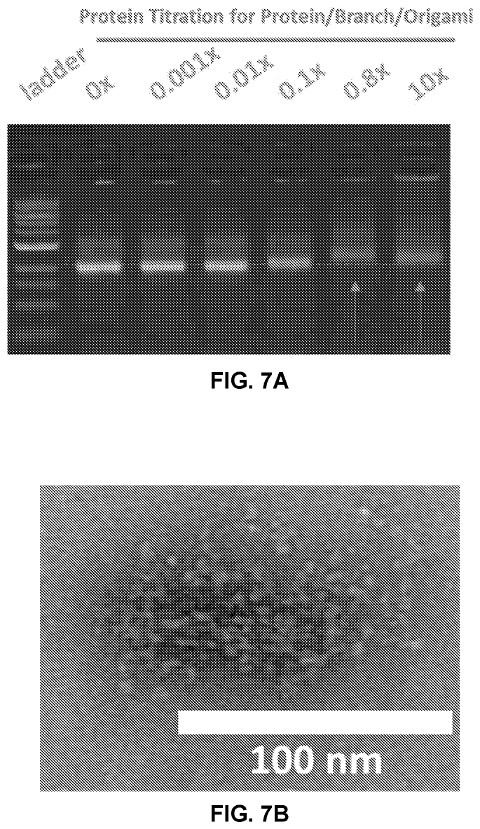

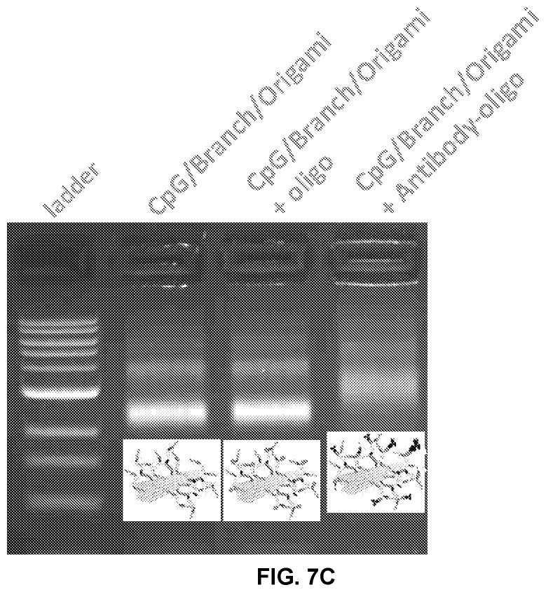

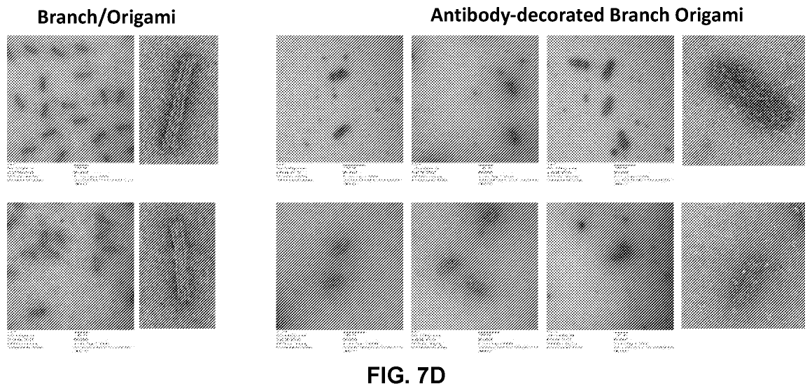

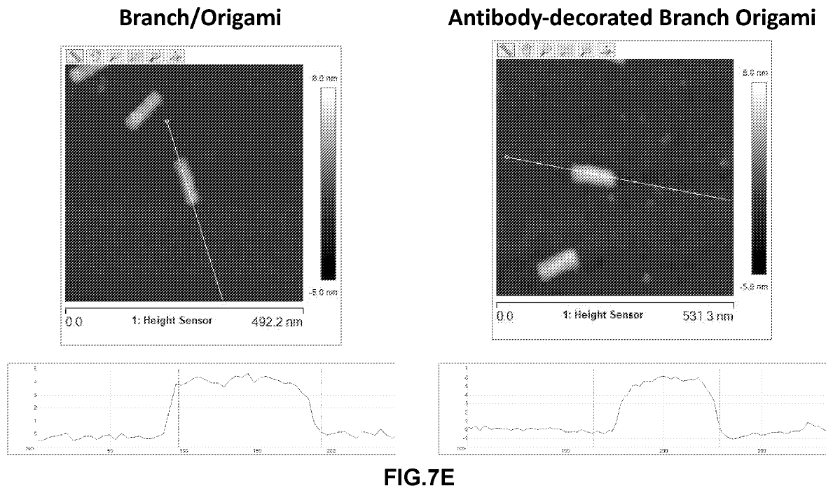

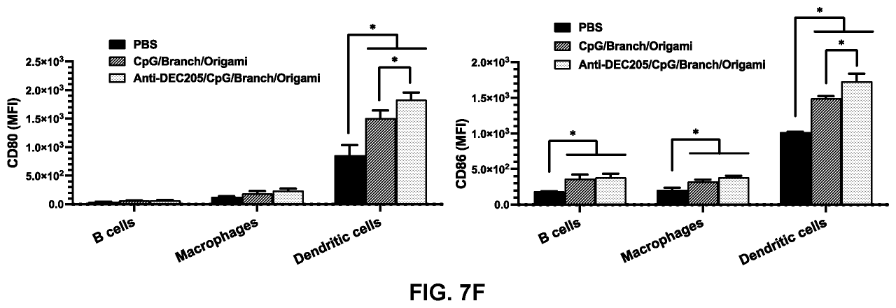

Example 7, illustrated by FIGS. 7A to 7F, shows precise and uniform attachment, as well as function of proteins on DNA branched origami coupled via branched attachment arms. FIG. 7A shows agarose electrophoresis gels, that based on the band shifts as indicated by the red arrow, demonstrate efficient, complete, and precise coupling of proteins onto the DNA branched origami, where sub equimolar ratio-based attachment resulted in nearly maximum attachment capacity. Note that tight bands on the gel for protein conjugated DNA branched origami indicate uniform and monodisperse product. FIG. 7B shows TEM image of DNA branched origami fully coated with ovalbumin protein as a model antigen, demonstrating high-density multivalent antigen presentation. FIG. 7C shows distinct shift in the gel band after electrophoresis for the antibody-oligonucleotide conjugate attachment to DNA branched origami loaded with CpG adjuvant; note that simply adding the oligonucleotide that was used for antibody conjugation did not result in the band shift. FIG. 7D shows TEM image of DNA branched origami on the left, and antibody decorated branched origami on the right. Since DNA branches on DNA origami are prone to collapsing during TEM sample preparation, making it difficult or impossible to visualize antibodies, AFM was performed (FIG. 7E) to demonstrate increase in thickness/height for the antibody-oligonucleotide loaded DNA branched origami as compared to oligonucleotide loaded DNA branched origami; approximately 1.5 nm increase in thickness was detected. Finally, FIG. 7E demonstrates enhanced adjuvancy, of CpG-loaded DNA branched origami specifically targets to DEC205 receptors as compared to non-targeted counterpart in splenocyte co-culture focusing on professional antigen presenting cells (B cells, macrophages, and dendritic cells); dendritic cells, which are the main cell population for DEC205 marker, were specifically targeted as indicated by dendritic cell-specific enhancement of upregulation of CD80 and CD86, co-stimulatory molecules necessary for T cell activation.

### Example 8

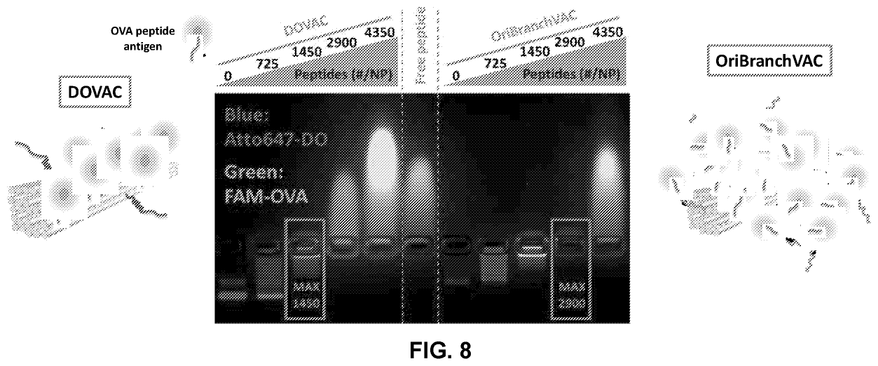

Example 8, illustrated by FIG. 8, shows fluorescence signal from agarose gel electrophoresis, demonstrating CpG (shown in blue) colocalization with DNA origami or DNA branched origami bands and high efficiency payload capacity of peptide antigen (shown in green) loading via electrostatic attachment as compared to free peptide band that migrates in the opposite direction of DNA structures on the gel due to the K10 positive charges. DNA branched origami maximum peptide loading was confirmed for up to 2900 antigen molecules per single branched origami NP (right side of the gel) and was enhanced as compared to a regular DNA origami peptide loading of 1450 antigen molecules per single origami NP (left side of the gel). This, and LL37-based peptide attachment allow for excluding purification steps due to 100% attachment efficiency, and require no additional modifications to the peptides except synthesizing peptides with additional amino acid sequences. Also, this methodology could be translated to protein attachment where proteins need to be expressed with one of the terminal ends containing LL37, or K, or R residues, or their combinations for electrostatic attachment to DNA-based NPs or other negatively charged nano-objects.

In some embodiments, proteins may be loaded onto DNA NPs by expressing DNA targeting antibody domain on native proteins to allow for binding of proteins to DNA NPs. Although many technologies have been described for DNA origami surface loading with peptides and proteins, such as oligonucleotide conjugation, none so far have the ability to cover the whole DNA origami surface with proteins and with such density as proposed herein. In terms of the protein loading, the disclosed method offers the same advantages as for the peptide loading. The results here demonstrate that for the first time it is possible to attach up to 1500-3000 antigenic peptides or more on the single plasmid-made DNA branched origami NP, depending on the design and size. So far, a maximum of 20-40 peptide attachments on DNA origami vaccine has been reported.

### Example 9

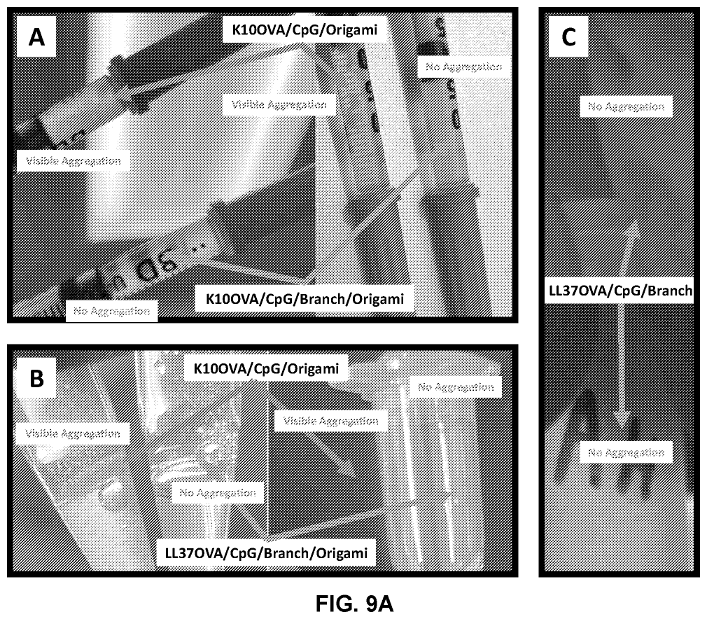

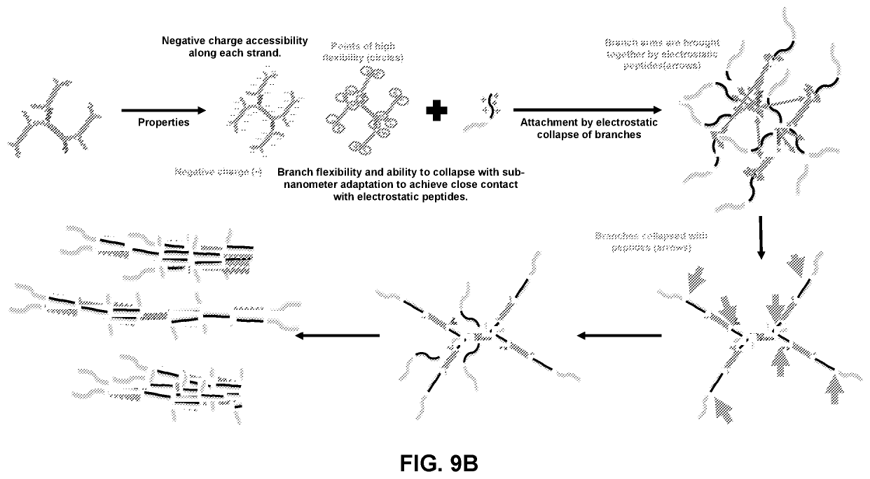

Example 9, illustrated by FIGS. 9A and 9B, unexpectedly and unpredictably, shows distinct differences in interaction of electrostatic peptides with DNA branched origami as compared to DNA origami. FIG. 9A show an image demonstrating appearance of DNA branched origami or DNA branches loaded with K10OVA or LL37OVA peptides as compared to regular DNA origami loaded with the same peptides. DNA branched origami NPs resist, reduce, or delay aggregation induced by electrostatic interactions between peptides and DNA NPs as compared to regular DNA origami, where aggregation is clearly visible upon mixing of electrostatic peptides and DNA origami. FIG. 9B shows a mechanism that may explain the unique interaction of branches or branch elements on DNA origami with electrostatic peptides. Depicted is an embodiment of nucleic acid branch properties in combination with nucleic acid-binding peptides giving rise to a novel means of peptide integration with nucleic acid branches; nucleic acid branches have negative charge, flexible waist and arms, and particularly important, multiple points of high flexibility, allowing for the attachment with nucleic acid-binding peptides by intra-branch electrostatic collapse of branches as depicted in the schematic. This unique and novel mechanism and predetermined folding points of high flexibility provide a degree of control over complexation process with peptides, shifting aggregation/complexation from inter-particle aggregation to intra-particle aggregation which may be more entropically favorable while satisfying at the same time favorable electrostatic charge neutralization of negative DNA charges with positive peptide charges. We believe that the unique potential ability of branches to predominantly cause intra-particle sandwiching of LL37 peptide sequences as opposed to inter-particle sandwiching, i.e., intra-particle aggregation is the underlying mechanism for this unexplained ability of branched structures in combination with LL37-peptide/protein. This is the first such concept ever introduced or proposed. In support of this, we have noticed on TEM and AFM that DNA branches, upon dispositioning on TEM grid or AFM mica surface, most often collapse or sandwich their structure within single particles as opposed to aggregating with other neighboring particles whether they are alone or attached to DNA origami, making it very difficult to image or capture DNA branches in their native non-collapsed form as they are found in the PBS. Hence, our intuition is that instead of electrostatic peptides causing complexation between two different nucleic acid NPs, the majority of the electrostatic complexation may be happening between the branching elements within the branching network of single DNA branch, or if on DNA origami, due to the dense packing of DNA branches on DNA origami, they are predominantly interacting through electrostatic peptides with each other within or on the single DNA branched DNA origami NP surface, hence reducing inter-particle aggregation. In essence, DNA branches are sequestering peptide charges, by folding them throughout the DNA branching structure. DNA branches also have the maximum surface area to allow the maximum interactions of peptides withing the network of a single DNA branch nanoparticle, instead bridging several DNA nanoparticles and causing aggregation or precipitation.

### Example 10

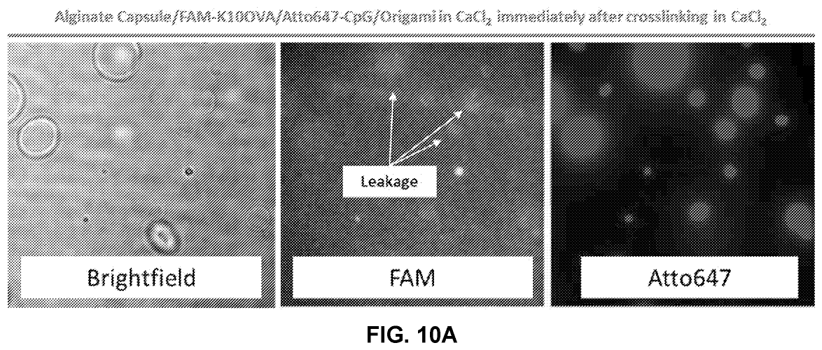

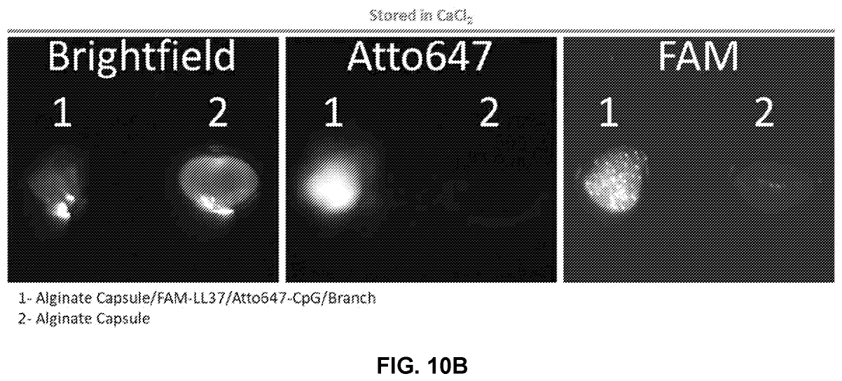

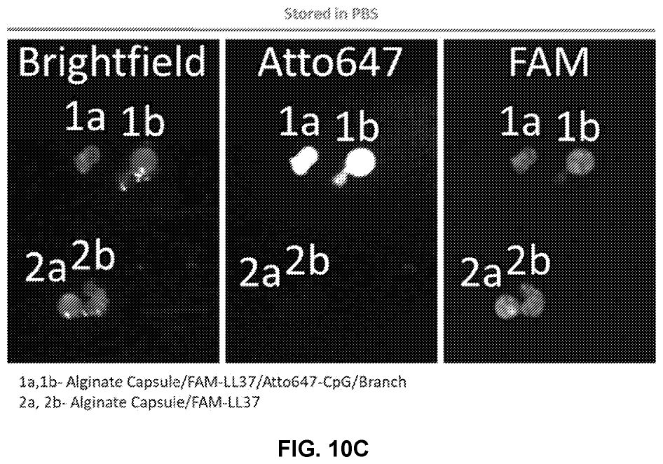

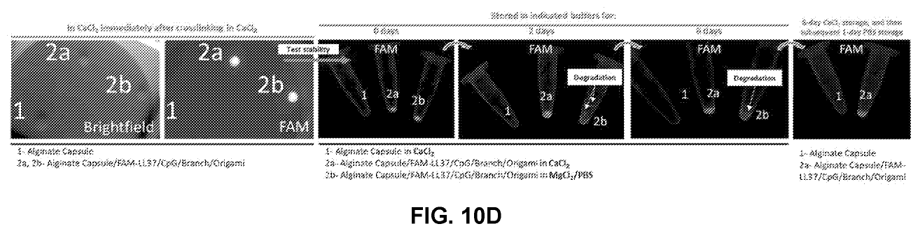

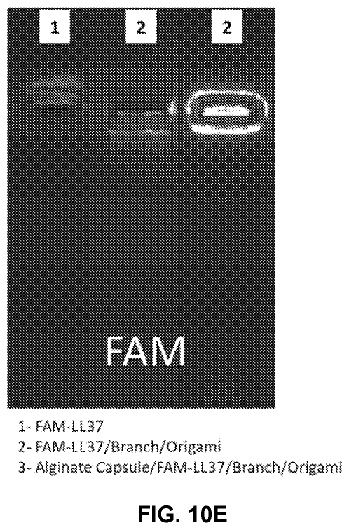

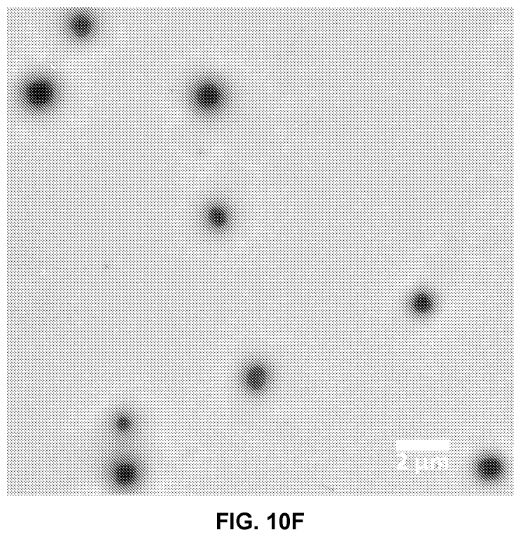

Example 10, illustrated by FIGS. 10A to 10F, shows, unexpectedly and unpredictably, a unique ability of LL37 peptide sequence-complexed DNA branch or DNA branched origami NPs to remain stable and retained within alginate capsules with the goal of controlled release and extended therapeutic activity as compared to K10 peptide sequence. FIG. 10A shows fluorescence microscopy image of alginate capsules loaded with FAM-K10OVA peptide complexed with Atto647-CpG-loaded origami, where K10-based OVA peptide completely leaked out of the capsules immediately after manufacturing and while in the crosslinking buffer, CaCl₂). FIG. 10B shows successful colocalization and retention of FAM-LL37 complexed with Atto647-CpG-loaded branches and encapsulated in alginate capsule as compared to alginate only control. Brightfield image of alginate capsule colocalized with the Atto647 and FAM signals for the alginate-encapsulated LL37/CpG/Branch. Interestingly, and very unpredictably, FIG. 10C shows that LL37 peptide was capable of being stably retained within alginate capsule on its own and when stored in PBS. FIG. 10D shows successful alginate encapsulation of FAM-LL37 complexed CpG-loaded branched origami in CaCl₂) buffer, as demonstrated by FAM signal colocalization (FAM fluorescence image) with alginate capsules (brightfield image). Furthermore, it is demonstrated that alginate capsules containing LL37/CpG/Branch/Origami are stable in CaCl₂) buffer, but not in 2.5 mM MgCl₂ buffer in PBS that is used to administer DNA origami or DNA branched origami formulations to cells or animals. In contrast, the same alginate capsules are stable in PBS for at least one day. FIG. 10E shows FAM fluorescence signal of agarose gel electrophoresis, demonstrating that DNA branched origami complexed FAM-LL37 peptides, as based on the shift of the band downward within the well as compared to free FAM-LL37 peptide shift upwards within the well. Alginate encapsulation of FAM-LL37 complexed branched origami was demonstrated by no shifting of the signal within the well. FIG. 10F shows TEM image of alginate-encapsulated LL37OVA and CpG-loaded DNA branched origami.

These alginate-based LL37-peptide complexed DNA branched and DNA branched origami formulations are not only useful for the traditional vaccine administration routes (e.g. S.C., I.M., oral), but also intranodal, and particularly important as potential cancer treatment route, intratumoral and peritumoral vaccinations. In this specific case, air-blast nozzle technique and CaCl₂) (a common technique) were used to produce microcapsules, but methods exist for production of nanocapsules as well. In some embodiments, the method further involves stabilization and crosslinking strategies of peptides with nucleic acid NPs.

### Example 11

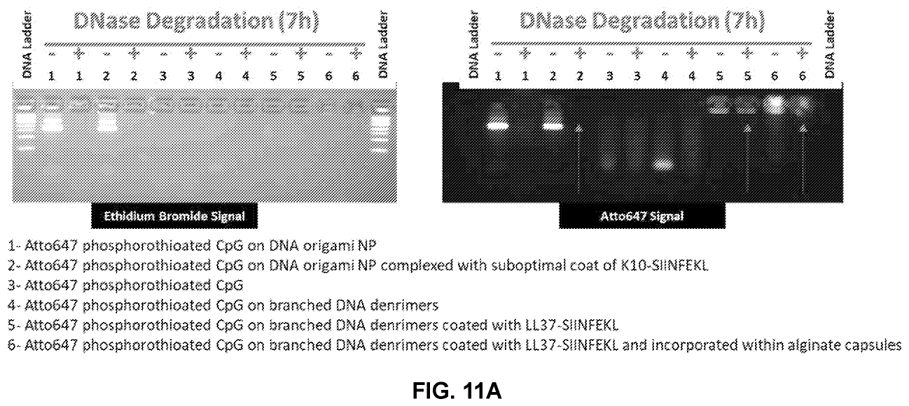

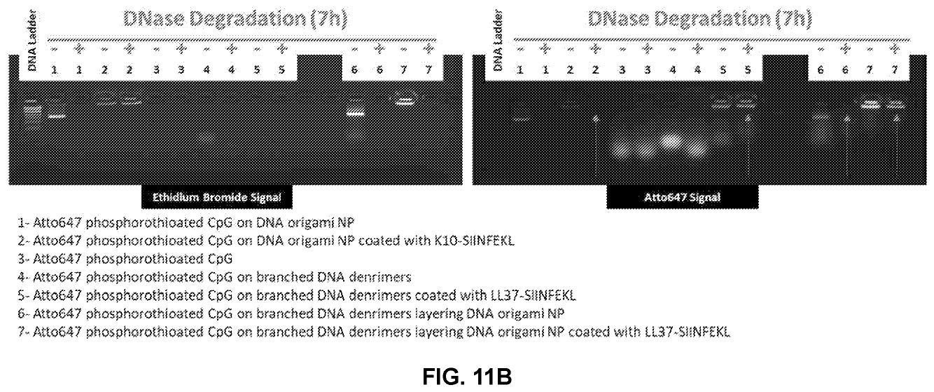

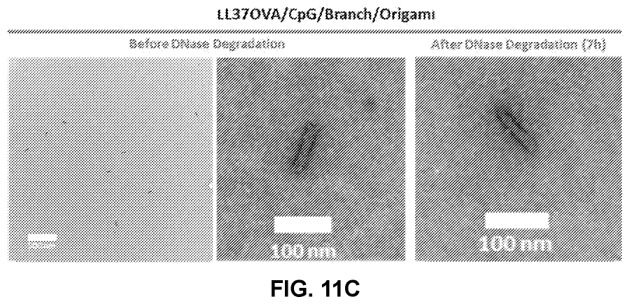

Example 11, illustrated by FIGS. 11A to 11C, demonstrates resistance of LL37OVA complexed DNA branches and DNA branched origami to DNase degradation (7-hour exposure at 0.5 U/μL DNase I and 0.5 μg/μL DNA), and unexpected advantage of LL37 peptide complexation system to protect the attachment of phosphorothioated CpG oligonucleotides on DNA nanostructures as compared to K10 counterpart. FIG. 11A shows agarose gel electrophoresis signal corresponding to DNA structures on the left and Atto647 signal corresponding to CpG adjuvant on the right. It was seen that at suboptimal K10OVA coating of CpG-loaded origami, neither structures, nor the attachment of CpG to origami were preserved upon incubation with DNase (red arrow). In contrast, complexing CpG-loaded DNA nanostructures, in this case DNA branches, with LL37OVA peptides preserved the attachment of CpG on DNA nanostructures (red arrow). Please note that due to the exceptional CpG loading capacity of DNA branches based on mass of CpG DNA loaded per mass of branch DNA, and to maintain similar Atto647 fluorescence signal of CpG among conditions, the signal of DNA branches in ethidium bromide channel is not easily detectable, especially after complexation with LL37OVA, and similar is true for the free CpG signal. Alginate encapsulation of LL37OVA complexed branches also maintained resistance to DNase degradation (red arrow). FIG. 11B demonstrates that while K10OVA coating protected DNA origami structures from DNase as demonstrated by the presence of DNA origami band in well 2 and absence of the band in well 1 under DNase treatment (ethidium bromide signal), K10OVA did not protect the attachment of phosphorothioated CpG onto DNA origami nanoparticles from DNase as demonstrated by the lack of band in the well 2 (Atto647 signal, red arrow). In contrast, LL37OVA protected not only DNA branches and DNA branched origami, but also the attached phosphorothioated CpG payload (red arrows). FIG. 11C shows TEM images demonstrating LL37OVA complexed CpG-loaded DNA branched origami structures had intact morphology after LL37OVA peptide coating and that LL37OVA protected structures from degradation.

### Example 12

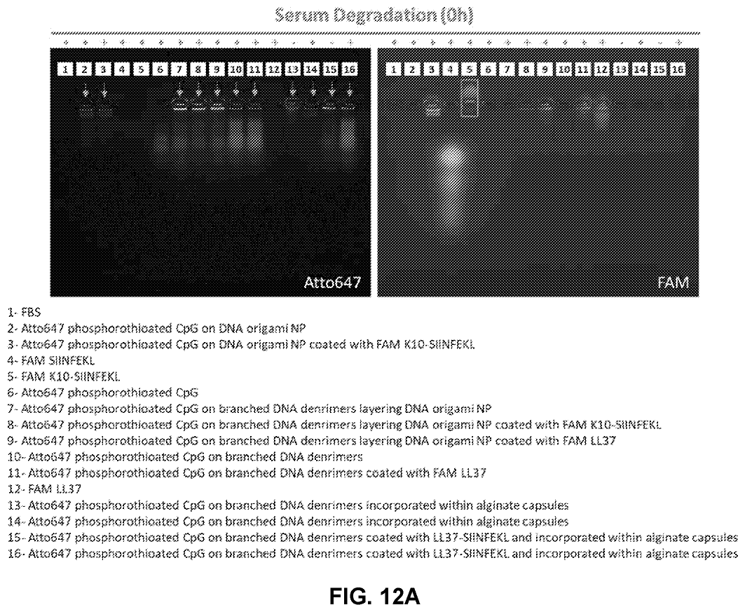

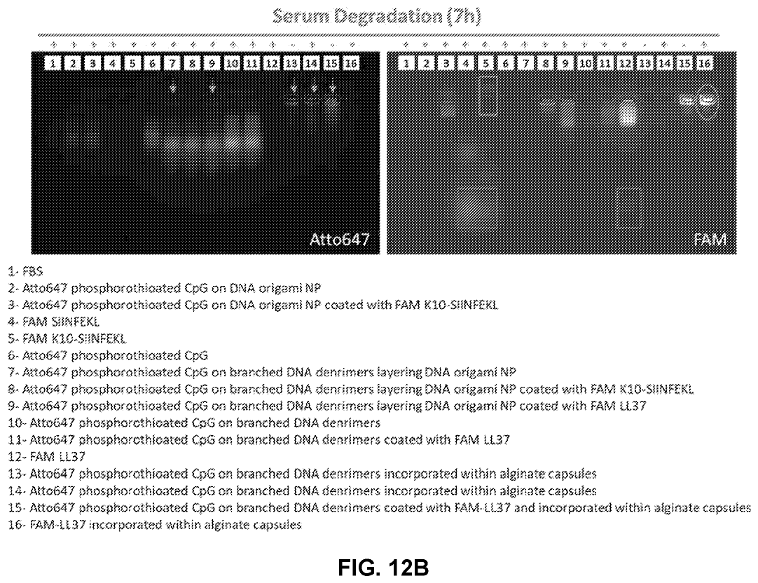

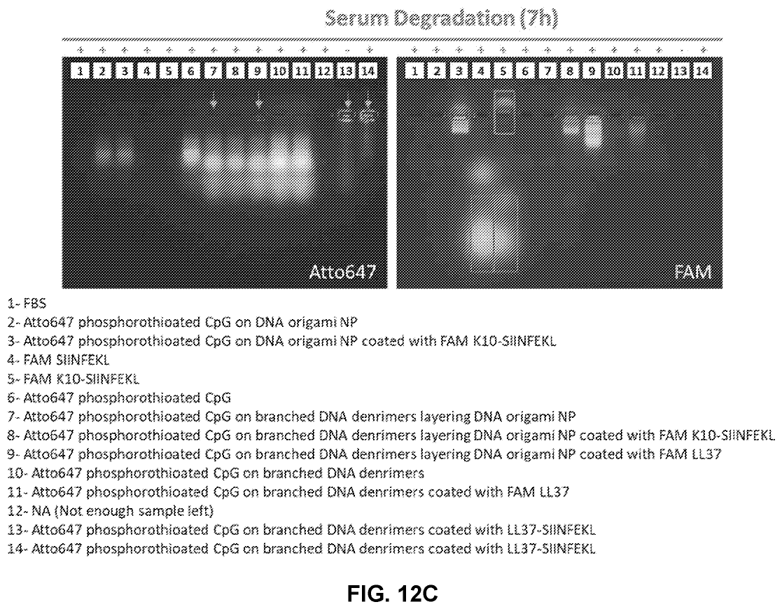

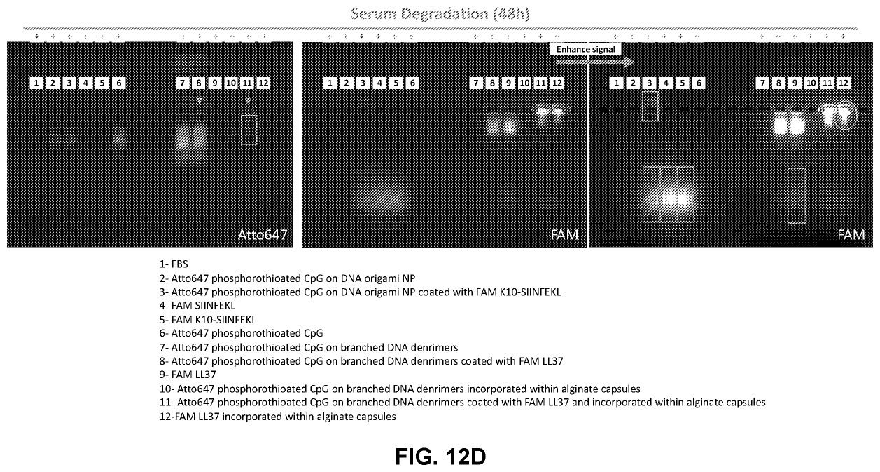

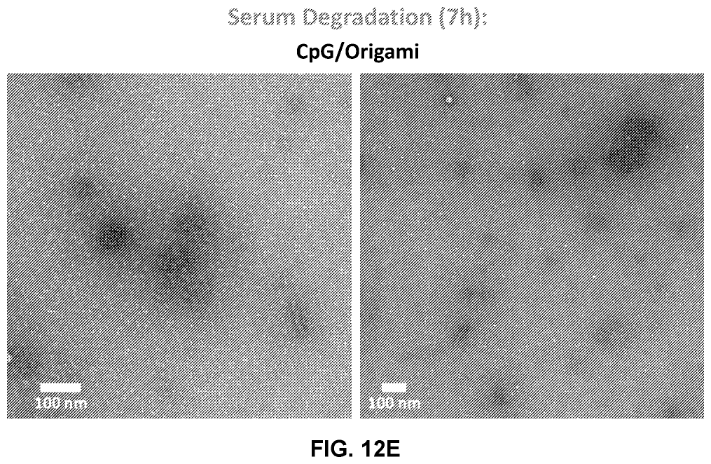

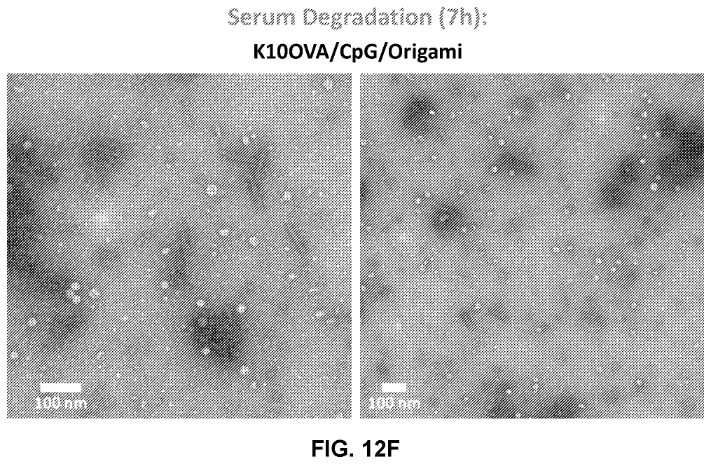

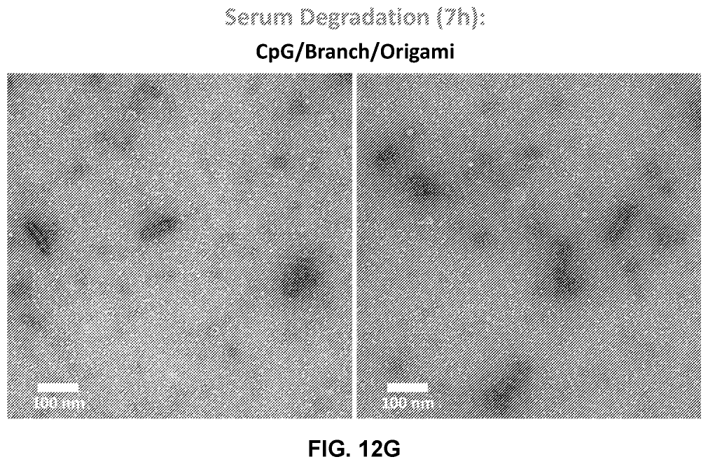

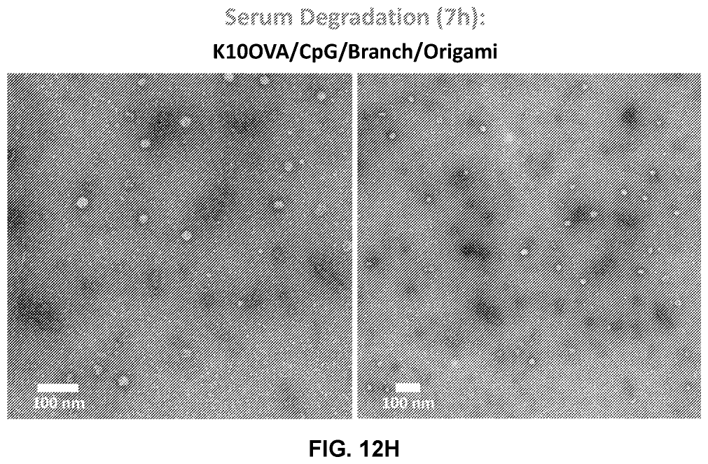

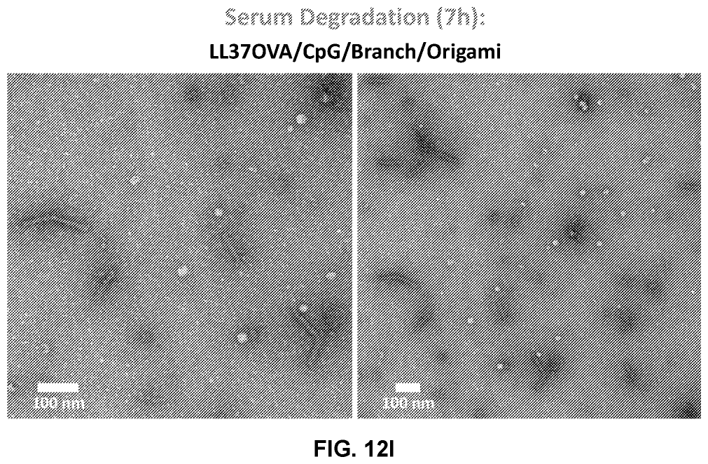

Example 12, illustrated by FIGS. 12A to 12I, demonstrates resistance of LL37OVA complexed DNA branches and DNA branched origami to degradation in 95% fetal bovine serum for various periods of time, and unexpected advantage of LL37 peptide complexation system to protect the attachment of phosphorothioated CpG oligonucleotides on DNA nanostructures as compared to K10 counterpart. The serum in these experiments was not heat-inactivated and was freshly thawed to preserve maximum degradation activity. FIG. 12A shows samples incubated with serum and immediately run on the gel as a reference representing no degradation. On the left is gel electrophoresis fluorescent image showing colocalization of CpG signal on DNA nanostructures in the wells as compared to free CpG signal (green arrows), while the gel on the right shows peptide signal (green square shows K10OVA peptide migrating in the opposite direction from DNA structures). FIG. 12B shows samples incubated with serum for 7 hours. The gel on the left shows Atto647 signal demonstrating that FAM-K10OVA did not help protect or retain CpG on the origami NP, while FAM-LL37 helped retain some CpG on the branched origami NPs (green arrows). Alginate fully protected CpG on branched DNA dendrimers complexed with FAM-LL37 peptides. The gel on the right shows FAM signal, demonstrating peptides remained complexed with DNA structures, and that DNA structures protected K10 or LL37 based peptides (red squares indicate degraded peptide components). Interestingly and unexpectedly, LL37 peptides on their own (i.e., without complexation with DNA structures) were stable within alginate capsules and did not leak or degrade even after 7 hours of incubation with serum. FIG. 12C shows another set of samples incubated with serum for 7 hours. The main difference in this gel as compared to gel in FIG. 12B is that non-FAM LL37OVA was shown to provide full protection of and/or compatibility with phosphorothioated CpG attachment (green arrows, wells 13 and 14) as compared to FAM-LL37 or FAM-K10OVA. FIG. 12D shows gel for the samples incubated with serum for 48 hours. The gel on the left shows Atto647 signal demonstrating that FAM-K10OVA did not help protect or retain CpG on the origami NP, while FAM-LL37 helped retain some CpG on the branched origami NPs (green arrows). Alginate fully protected CpG on branched DNA dendrimers complexed with FAM-LL37 peptides (green arrow), while some CpG loaded branches were pulled out from alginate bead during gel electrophoresis (green square), demonstrating alginate beads sequestered formulations from interaction with serum for at least 48 hours. The gel on the right shows FAM signal demonstrating peptides completely degraded after 48 hours in serum (red squares), except FAM-LL37 peptide which appeared to have a low level of degradation. Unexpectedly, branched DNA dendrimers protected LL37 as compared to LL37 alone. Again, alginate encapsulation fully protected LL37 peptides complexed with CpG-loaded DNA branches. Very unpredictably and unexpectedly, LL37 peptide was protected from degradation and stable (no leakage) within alginate capsule even after 48 hours. FIG. 12E is a TEM image demonstrating that the majority of CpG-loaded DNA origami were degraded after degradation in serum for 7 hours. FIGS. 12F to 12I are TEM images demonstrating different levels of DNA NP degradation after 7 hours in serum, with many intact structures visible for LL37OVA coated CpG-loaded DNA branched origami structures.

### Example 13

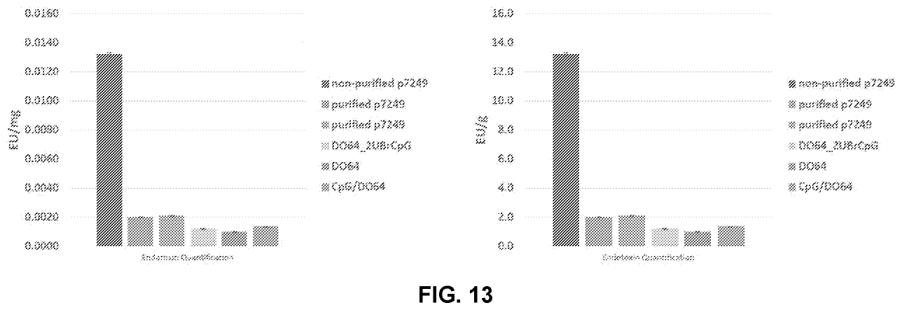

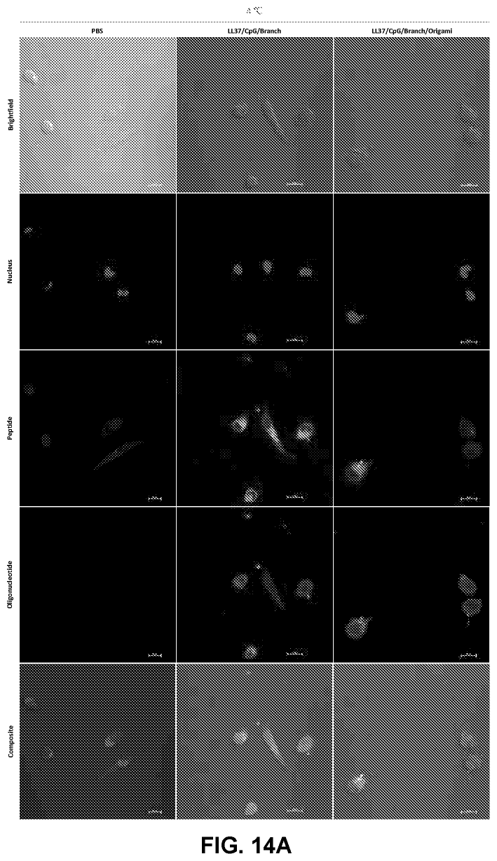

Example 13, illustrated by FIG. 13, demonstrates successful purification of endotoxin down to 2 EU/g or below for p7249 scaffolds and DNA structures used as components of vaccines. To note is that alginate used in this project was low endotoxin alginate, and this version is commonly used in research towards biomedical applications and for vaccine development.

### Example 14

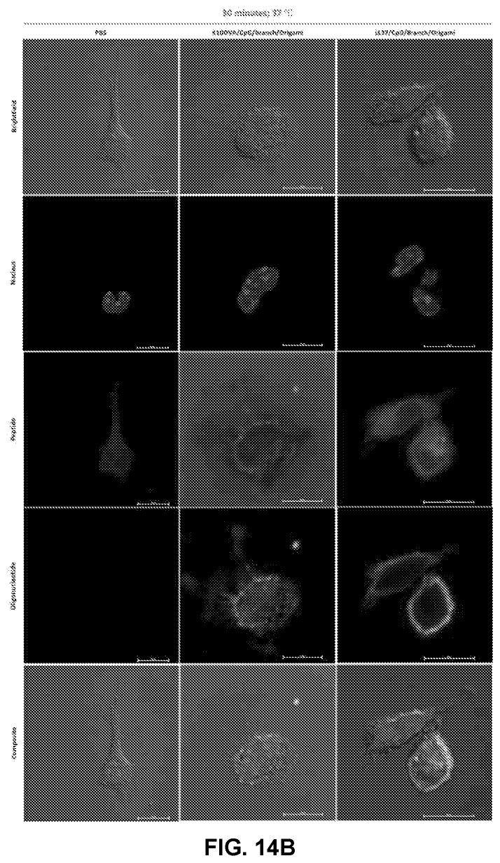

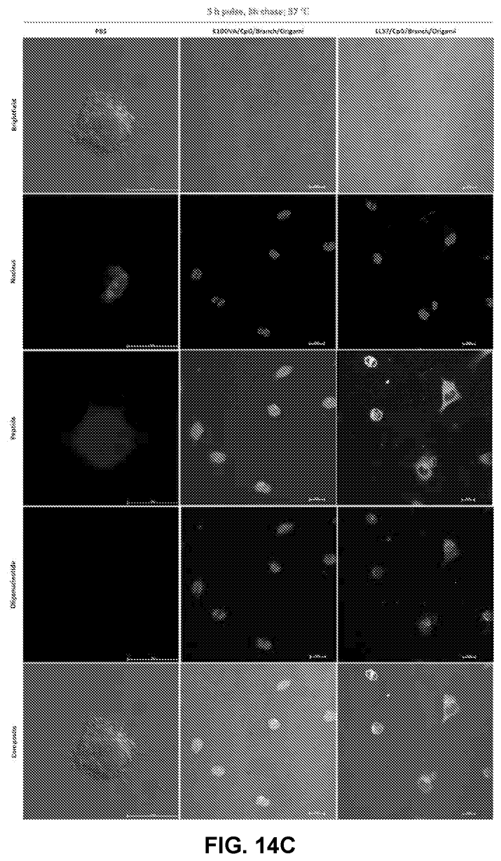

Example 14, illustrated by FIGS. 14A to 14C, shows confocal fluorescence images demonstrating binding and uptake of CpG-loaded DNA branched origami complexed with K10- or LL37-based peptides in peritoneal macrophages isolated from C57/B16 WT mice. Cells were plated on gelatin-coated coverslips as a mimic of extracellular matrix, and upon attachment, formulations were administered in complete medium containing 10% heat inactivated fetal bovine serum. FIG. 14A demonstrate that LL37 complexed DNA structures, either CpG-loaded DNA branches or DNA branched origami, bound only to cells and not the gelatin-coated coverslips as compared to PBS control. Although incubated at 4° C., where active cell uptake is prevented, unexpectedly, a profuse cytosolic signal was still detected for both, the peptide and CpG oligonucleotide components for both, DNA branches and DNA branched origami structures complexed with LL37 peptide. Images also demonstrated colocalization of peptide and oligonucleotide components. FIG. 14B compares LL37- and K10-based peptide attachment system to DNA branches after 30 minutes of incubation at 37° C. While both systems resulted in oligonucleotide signal located on cells only, and some colocalization with peptide signal, unexpectedly, only LL37-based attachment resulted in peptide delivery specifically to cells, while K10-based attachment resulted also in non-specific binding to gelatin-coated coverslips. FIG. 14C compares LL37- and K10-based peptide attachment system to DNA branched origami. Cells were incubated with formulations for hours (pulse), and then media replaced with fresh media lacking formulations for 3 hours (chase) at 37° C. (8 hours total incubation time). While both systems resulted in oligonucleotide signal located on cells only, and formulations were internalized based on their location within the cells, again, only LL37-based attachment resulted in peptide delivery specifically to cells, while K10-based attachment resulted also in non-specific binding of peptides to gelatin-coated coverslips. To note is that some punctuate signal was also seen on the coverslips for LL37 complexed DNA NP formulation, but here the peptide and oligonucleotide signals were colocalized as compared to K10-based peptide signal which was profuse throughout the whole coverslip appearing as free peptide; some level of NP binding to gelatin-coated coverslips is expected.

### Example 15

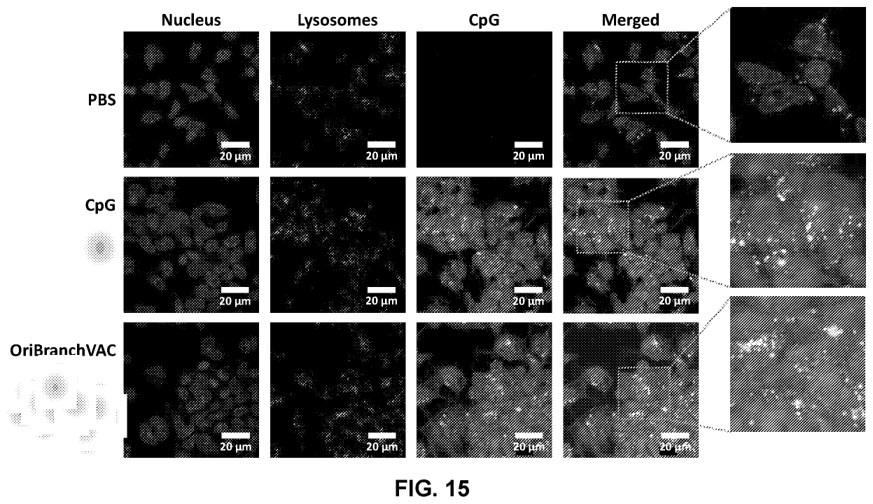

Example 15, illustrated by FIG. 15, shows a confocal fluorescence image demonstrating colocalization of CpG-loaded DNA branched origami with phagolysosomes or lysosomes and cytosolic delivery ability. MUTUDC 1940 dendritic cell line was plated on gelatin-coated coverslips, cells were pulsed with dextran (shown in red) for 45 minutes, media replaced for 45 minutes to allow accumulation of dextran in lysosomes or phagolysosomes, and then treated with PBS, CpG, or CpG-loaded DNA branched origami. Images demonstrate that CpG-loaded DNA branched origami does not hinder delivery of CpG to lysosomes or phagolysosomes, or cytosolic escape of oligonucleotides (e.g., CpG).

### Example 16

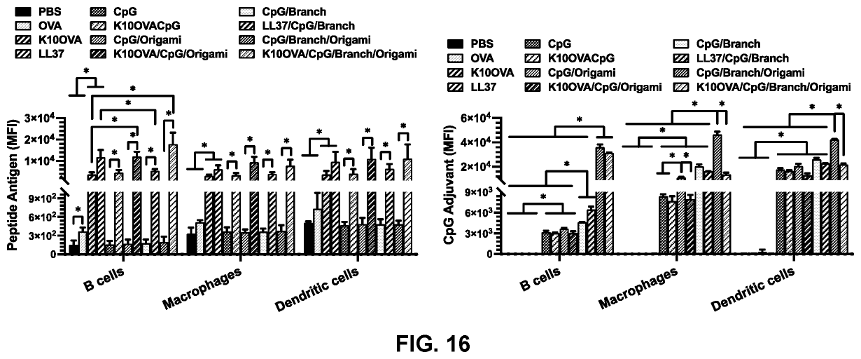

Example 16, illustrated by FIG. 16, shows flow cytometry results demonstrating enhanced uptake of peptide antigens and phosphorothioated CpG oligonucleotide adjuvants for various DNA NP-based delivery systems utilizing or not K10- and LL37-based peptide attachment technology administered to professional antigen presenting cells in splenocyte co-culture (after isolation from C57/BI6 WT mice and red blood cell lysis and removal). Complete media containing 10% heat inactivated fetal bovine serum was used. Please note that the OVA here stands for SIINFEKL peptide, K10OVA stands for KKKKKKKKKKSIINFEKL, and LL37 stands for human LL37, LLGDFFRKSKEKIGKEFKRIVQRIKDFLRNLVPRTES. The graph on the left demonstrates that both regular origami and branched origami increase delivery of peptides to B cells, while the graph on the right demonstrates that unexpectedly DNA branched origami strongly enhances the delivery of CpG as compared to DNA branch or DNA origami counterparts, although all three formulations (i.e., DNA branches, DNA origami, and DNA branched origami) enhance the delivery of CpG as compared to free CpG. Interestingly, K10-based attachment reduces phosphorothioated CpG delivery, particularly in macrophage and dendritic cell populations, while LL37 causes no difference in macrophage and dendritic cell populations, but appears to enhance delivery in B cells. This reduction in delivery by K10-based system may be due to K10 perhaps inducing de-coupling of CpG from DNA NPs as demonstrated in DNase and serum degradation studies, while unexpectedly, LL37 does not have the same effect.

### Example 17

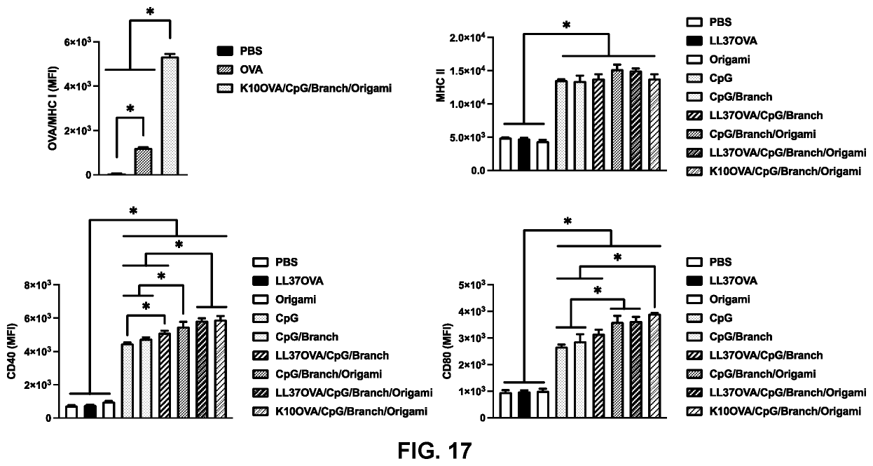

Example 17, illustrated by FIG. 17, shows flow cytometry results demonstrating enhanced peptide antigen presentation via MHCI and activation via upregulation of co-stimulatory molecules in MUTUDC1940 dendritic cell line by DNA branch- and DNA branched origami-based vaccine formulations incubated in complete media containing 10% heat inactivated fetal bovine serum. Please note that the OVA here stands for SIINFEKL peptide, K10OVA stands for KKKKKKKKKKSIINFEKL, and LL37OVA stands for LLGDFFRKSKEKIGKEFKRIVQRIKDFLRNLVPRTESSIINFEKL, where LL37 portion of this chimeric peptide is a human version of the sequence. Top left figure demonstrates enhanced antigen presentation of K10-based OVA peptide complexation with CpG-loaded DNA branched origami as compared to OVA peptide alone. Please note that flow cytometry antibody was used that specifically detects SIINFEKL peptide complexed with MHCI, but not SIINFEKL peptide alone or bound elsewhere on cell or NP surface. Top right figure demonstrates MHCII upregulation by all formulations containing CpG, but not DNA origami alone. Bottom left figure demonstrates that all CpG-containing formulations induce upregulation of CD40 co-stimulatory molecule as compared to PBS control, LL37OVA peptide, or DNA origami alone. Furthermore, it demonstrates that although LL37OVA peptide attachment technology does not stimulate dendritic cells by itself, it enhances dendritic cell stimulation of CpG-loaded DNA branches. It also demonstrates that DNA branching design of DNA origami also enhances dendritic cell stimulation as compared to CpG alone, and this is further enhanced by LL37- or K10-based peptide attachment system. Importantly, this figure also demonstrates advantage of CpG-loaded DNA branched origami design stimulation over CpG-loaded DNA branch design alone. Bottom right figure demonstrates similar trends in enhancements of upregulation of co-stimulatory molecule CD80 as for CD40.

### Example 18

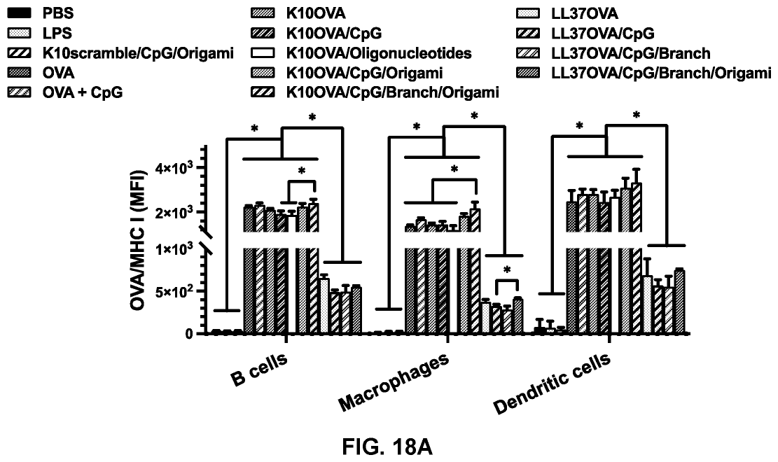

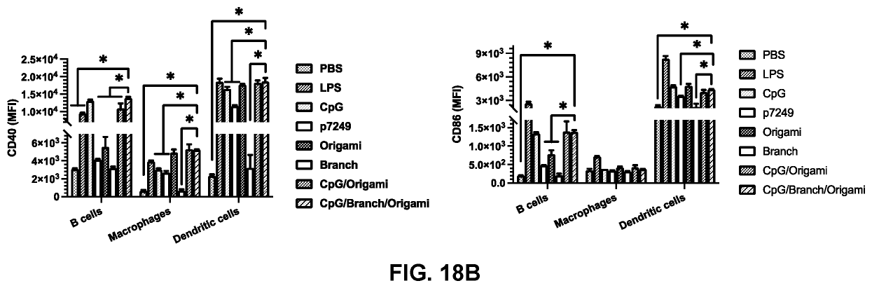

Example 18, illustrated by FIGS. 18A and 18B, shows flow cytometry results demonstrating peptide antigen presentation via MHCI and activation via upregulation of co-stimulatory molecules induced by various DNA NP-based delivery systems utilizing or not K10- and LL37-based peptide attachment technology administered to professional antigen presenting cells in mouse splenocyte co-culture (after isolation from C57/B16 WT mice and red blood cell lysis and removal). Complete media containing 10% heat inactivated fetal bovine serum was used. Please note that the OVA here stands for SIINFEKL peptide, K10OVA stands for KKKKKKKKKKSIINFEKL, and LL37OVA stands for LLGDFFRKSKEKIGKEFKRIVQRIKDFLRNLVPRTESSIINFEKL, where LL37 portion of this chimeric peptide is a human version of the sequence. FIG. 18A demonstrates successful antigen processing of K10- and LL37-based antigen peptides. It also demonstrates the advantage of using DNA branched origami-induced peptide delivery to enhance antigen presentation as compared to DNA branch or DNA origami counterparts. Please note that LL37-based antigen peptide delivery resulted in lower SIINFEKL presentation as compared to the native SIINFEKL, and this may have been either due to inherent peptide design differences or differences in peptide solubility and suboptimal dissolution/reconstitution of peptides due to the inexperience and novelty of LL37-based chimeric polypeptides which behave very differently than LL37 peptides alone or SIINFEKL peptides alone. FIG. 18B demonstrates upregulation of co-stimulatory molecules CD40 and CD86, where CpG-loaded DNA branched origami design is advantageous in stimulating CD40 in macrophages and dendritic cells as compared to CpG-loaded DNA branch design and CpG alone, while DNA branches do not stimulate cells and are non-immunogenic. DNA origami itself does stimulate cells and is relatively immunogenic. Due to complexity of showing statistics in this graph, statistics are shown only comparing CpG-loaded DNA branched origami vs. all other conditions.

### Example 19

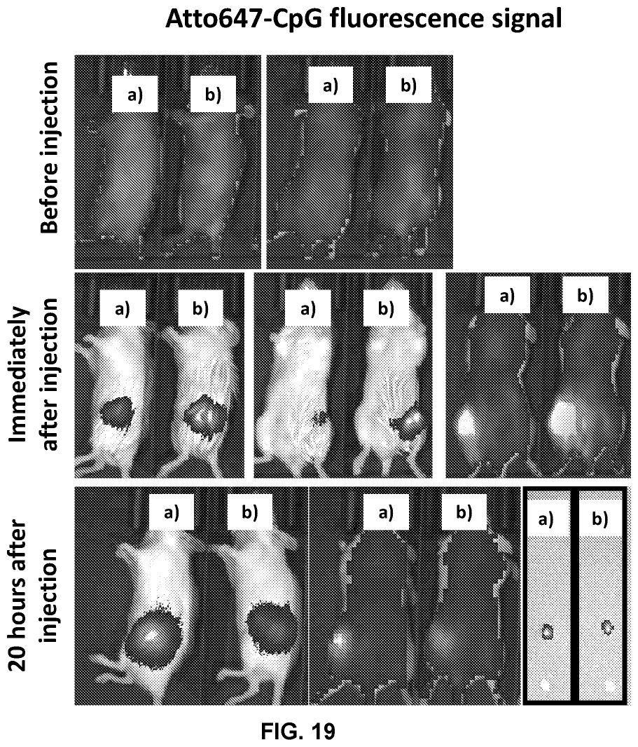

Example 19, illustrated by FIG. 19, shows biodistribution of Atto647-CpG-loaded and LL37OVA coated DNA branches as compared to of Atto647-CpG alone after I.M. injection in albino C57/B16 mice using IVIS imaging. Mouse a and b were imaged in different positions before injections to demonstrate background signal and subsequently injected with either Atto647-CpG-loaded DNA branches complexed with LL37OVA peptide (mouse a), or Atto647-CpG alone (mouse b). Images were taken immediately or hours after injection. It was noticed, similar as with fluorescence gel imaging that Atto647 signal on CpG was attenuated by LL37OVA peptide coating. Hence, the whole-body images demonstrate stronger initial signal (immediately after injection) for CpG alone as compared to the DNA branch-based vaccine, despite injection of the same Atto647-CpG dose. However, 20 hours after injection, the whole-body images demonstrate a very strong signal for the Atto647-CpG-loaded DNA branches complexed with LL37OVA peptide (mouse a), while the signal for the Atto647-CpG alone is very weak. In addition, the signal for the Atto647-CpG-loaded DNA branches complexed with LL37OVA peptide (mouse a) is very localized and appears to be completely in the lymph node, while the signal for Atto647-CpG alone (mouse b) is more diffused. Indeed, after collection of the draining lymph nodes (top) as compared to non-draining lymph nodes (bottom), it was verified that the formulations are specifically delivered to the draining lymph nodes as intended. On the other hand, it was observed that the K10-based peptide delivery system takes at least 3 days to deliver DNA NP-based formulations to draining lymph nodes, perhaps due to very strong non-specific binding of K10 to tissues (data not shown). Please note that OVA indicates SIINFEKL in this example.

### Example 20

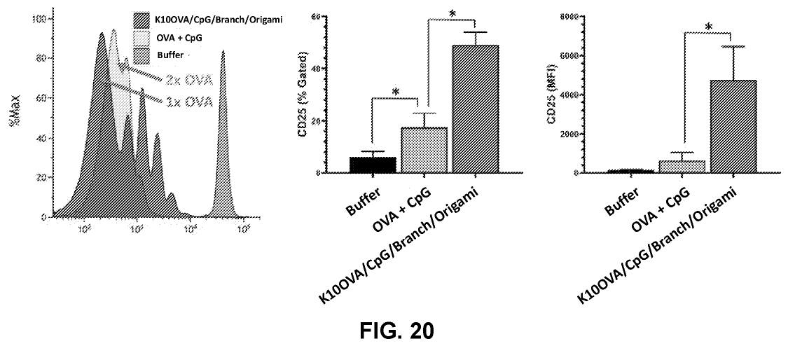

Example 20, illustrated by FIG. 20, shows flow cytometry results demonstrating that K10OVA-complexed and CpG-loaded DNA branched origami enhances SIINFEKL-specific T cell expansion and activation, as compared to mixture of OVA+CpG or PBS buffer control, 3 days after injection of indicated samples via S.C. route in C57/B16 WT mice that previously received adoptive transfer of SIINFEKL specific- and Cell Trace Violet-labeled T cells from OT-1 mice. This is despite OVA being at double the dose in the OVA+CpG mixture as compared to DNA branched origami vaccine (graph on the left). From the kinetics of the T cell expansion, it is evident that DNA branched origami vaccine delivery technology has delayed and prolonged immune stimulation (graph on the left). CD25 activation marker on T cells demonstrates that this stimulation lasts for at least 3 days, and is significantly enhanced as compared to the OVA+CpG mixture or PBS buffer control (graphs in the middle and on the right). This in part may be due to K10-induced nonspecific tissue interactions and delayed drainage to lymph nodes, as mentioned above in the biodistribution study (FIG. 19). Please note that OVA indicates SIINFEKL in this example.

### Example 21

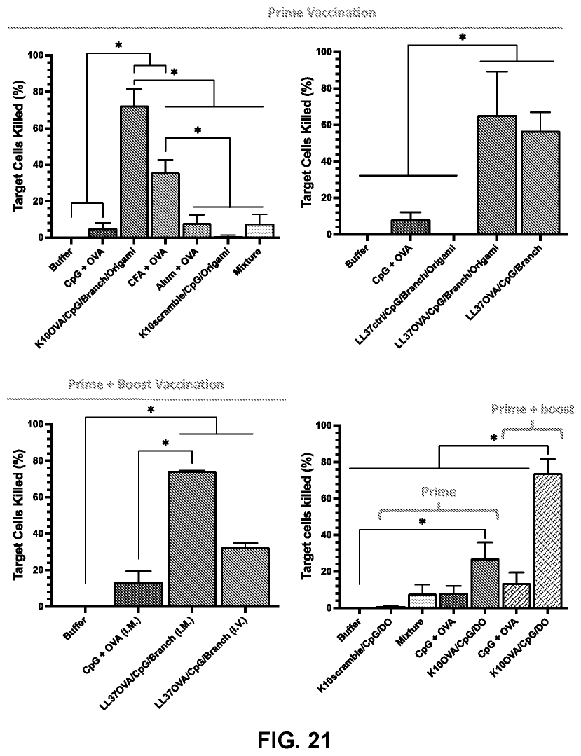

Example 21, illustrated by FIG. 21, shows flow cytometry results demonstrating enhanced antigen-specific target cell killing induced by CpG-loaded DNA branched DNA origami or DNA branches complexed with either K10- or LL37-based SIINFEKL delivery systems via various administration routes. A standard in vivo T cell cytotoxic killing assay was used. Briefly, in these experiments, C57/BI6 WT mice were vaccinated with one vaccine dose (prime vaccination) or two vaccine doses separated 2 weeks apart (prime+boost vaccination) or administered PBS, as indicated in the graphs, followed by I.V. injection of differentially stained target (SIINFEKL-pulsed and Cell Trace Violet low signal intensity-labeled) and non-target (un-pulsed and Cell Trace Violet high signal intensity-labeled) splenocytes (after red blood cell lysis and removal) 7 days post last vaccination. 24 hours later, spleens were isolated and red blood cells removed to measure the two Cell Trace Violet-labeled cell populations (i.e., the high and the low Cell Trace Violet fluorescence signal intensity populations). The percent killing was calculated using standard equations, based on the target and non-target populations in each condition and in reference to control PBS (untreated) mice where no antigen specific killing is present. Percentages of the two populations (high and low fluorescence intensity populations) were also verified each time before I.V. injection. Top left figure demonstrates that, unexpectedly, CpG-loaded and K10SIINFEKL complexed DNA branched origami, upon a single S.C. dose, induces significantly higher antigen-specific target cell killing as compared not only to the mixture of CpG+OVA or mixture of CpG+OVA+DNA origami, but also gold standard subunit vaccine references based on the Complete Freund's Adjuvand (CFA) and alum. Alum is currently used in many human vaccines, while CFA is not used in humans due to reactogenicity. The DNA branched origami formulation with a scramble version of K10OVA peptide does not induce killing as expected. All formulations presented in this example were S.C. and single injections. Top right figure demonstrates that, upon a single dose via I.M. route, LL37-based SIINFEKL attachment to either CpG-loaded DNA branches or DNA branched origami induces significantly higher antigen-specific target cell killing as compared to the mixture of OVA+CpG or LL37 control peptide complexed with CpG-loaded DNA branched origami formulation. Surprisingly and unexpectedly, the bottom left figure demonstrates that LL37-based attachment technology is suitable and capable, when complexed to CpG-loaded DNA branches, to induce significant antigen-specific T cell killing of target cells via I.V. route vaccination. Furthermore, the prime+boost regimen results in improved and more consistent responses as compared to prime only vaccination for I.M. injected LL37OVA-complexed and CpG-loaded DNA branches. The bottom right figure shows prime, as well as prime+boost vaccination for a regular CpG-loaded origami complexed with K10OVA peptides, demonstrating that significant improvement of this formulation over the mixture of CpG+OVA was achieved only after the prime+boost dose. This result indicates enhanced performance of DNA branched origami and LL37-peptide attachment technology. All the formulations contained the dose per each mouse, where applicable, as following: 0.01 nmol DNA origami, 0.64 nmol CpG, and 7.25 nmol antigenic peptide. CFA was prepared by mixing the ratio of 7.25 nmol SIINFEKL in 50 μL PBS with 50 μL CFA; larger quantity was prepared and properly emulsified to allow drawing of 100 μL in each syringe; it was verified that the emulsion was stable at least for several months by setting aside an aliquot and observing over time. Alum-based vaccine was prepared by mixing 7.25 nmol SIINFEKL in 50 μL PBS with 50 μL of 2% aluminum hydroxide (Alhydrogel).

### Example 22

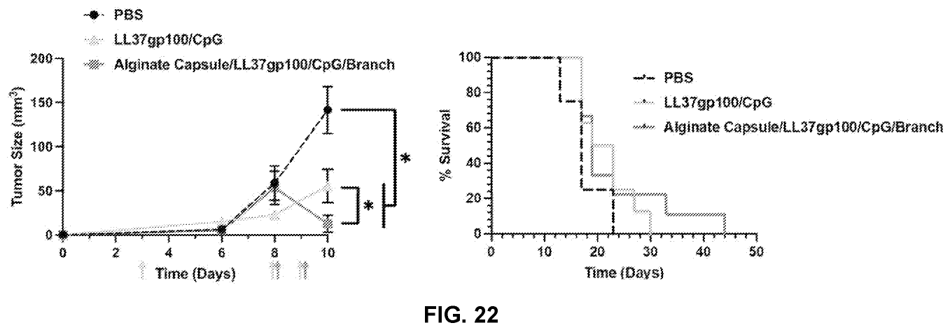

Example 22, illustrated by FIG. 22, shows therapeutic anti-tumor efficacy of LL37-gp 100 complexed with CpG and enhanced therapeutic efficacy of alginate capsules containing LL37gp100-complexed and CpG-loaded DNA branches against B16-F10 melanoma tumor model in C57/BI6 WT mice. Mice were injected S.C. with 2.5×10⁵ B16-F10 cells in 50 μL of PBS on day 0, weighed and monitored for tumor growth. On day 3, one group (shown in green, LL37gp100/CpG group) of mice was vaccinated via I.M. route with LL37gp100 complexed with CpG; the dose of CpG was 1.28 nmol/mouse and 20 nmol LL37gp100 peptide. On days 8 and 9, intratumoral injections were performed as indicated: LL37gp100/CpG group received LL37gp100/CpG, while Alginate Capsule/LL37gp100/CpG/Branch group received Alginate Capsule/LL37gp100/CpG/Branch, where the dose for each injection was 0.64 nmol CpG and 10 nmol LL37gp100 per each mouse. Please note that the two subsequent intratumoral injections were performed, where the dose was split between the two days, due to the difficulty of intratumoral injections using peptide volumes as reconstituted in this study. Figure on the left demonstrates, very unexpectedly and very unpredictably, surprising and rapid anti-tumor efficacy of Alginate Capsule/LL37gp100/CpG/Branch formulation upon only 2 subsequent doses, which was significantly better than the efficacy of LL37gp100/CpG complex which was administered as 3 total doses (4 dose equivalent to the alginate formulation) or PBS/untreated mice. Both, LL37gp100/CpG complex and Alginate Capsule/LL37gp100/CpG/Branch demonstrated significant reduction in tumor growth. Figure on the right demonstrates improved survival for both the LL37gp100/CpG complex and Alginate Capsule/LL37gp100/CpG/Branch formulations as compared to PBS/untreated mice. These results are particularly unexpected and can not be explained by the dosages of each therapeutic component separately, as for example, in Amaria, et al. “2013-0422: INDUCTION OF ANTITUMOR RESPONSE IN MELANOMA PATIENTS USING THE ANTIMICROBIAL PEPTIDE LL37.”, a single intratumoral injection containing 20 μmol of LL37 peptide and 20 μmol of CpG adjuvant or not were used to treat tumors in B16 melanoma model. Here, our alginate formulation contained 3 orders of magnitudes smaller cumulative dosages of LL37 chimeric peptides or CpG, totaling to 20 nmol and 1.28 nmol, respectively. This indicates that our alginate/LL37 chimera/CpG/DNA branch formulation has a unprecedented efficacy at low dosage, which is expected to greatly reduce the side effects of this immunotherapy approach.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A nanoparticle comprising i) a base nanostructure formed from a plurality of nucleic acid scaffold strands and a plurality of nucleic acid staple strands assembled into a geometry, ii) a branched oligonucleotide dendrimer,
wherein the base nanostructure is a non-branching and non-dendrimer nucleic acid nanostructure,
wherein the base nanostructure comprises one or more first single stranded nucleic acid oligonucleotide attachment arms configured to directly bind to a first complementary nucleic acid oligonucleotide strands, and
wherein the branched oligonucleotide dendrimer that has self-assembled by nucleic acid oligonucleotide complementarity and non-complementarity that comprises i) a plurality of second single stranded nucleic acid oligonucleotide attachment arms configured to bind to second complementary nucleic acid oligonucleotide strand, and ii) at least one of the first complementary nucleic acid oligonucleotide strands.

2. The nanoparticle of claim 1, further comprising a peptide attached to the base nanostructure and/or the branched oligonucleotide dendrimer by electrostatic interaction.

3. The nanoparticle of claim 2, wherein the peptide comprises i) a peptide antigen sequence, cell penetrating peptide sequence, or ligand-targeted peptide sequence, and ii) a charged peptide sequence configured to attach to the nanoparticle by electrostatic interactions.

4. The nanoparticle of claim 3, wherein the charged peptide sequence comprises at least 5, 6, 7, 8, 9, or 10 contiguous positively charged amino acids.

5. The nanostructure of any one of claims 2 to 4, wherein peptide comprises the amino acid sequence LLGDFFRKSKEKIGKEFKRIVQRIKDFLRNLVPRTES (SEQ ID NO: 1).

6. The nanoparticle of any one of claims 2 to 5, comprising at least 1,000 peptides attached to the base nanostructure and/or the branched oligonucleotide dendrimer.

7. The nanoparticle of any one of claims 2 to 5, comprising at least 50, 60, 70, 80, 90, 100 nM peptide.

8. The nanoparticle of claim 1, consisting of nucleic acids and ions.

9. The nanoparticle of any one of claims 1 to 8, further comprising a nucleic acid-based adjuvants.

10. The nanoparticle of claim 9, wherein the nucleic acid-based adjuvant is a CpG adjuvant.

11. The nanoparticle of any one of claims 1 to 10, wherein the base nanostructure is completely or partially assembled using DNA or RNA origami to direct and organize the origin of nucleic acid-made branches on or within the base nanostructure using exclusively nucleic acids material and the 3D nature of the base nanostructure and branches themselves, while nucleic acid-based branches further control the topography and density of the structure, including based on the length of branching units and the branching density or frequency, including the topography, density, and spacing of attachment sites for various functions and various molecules of interest.

12. The nanoparticle of any one of claims 1 to 11, wherein each branched oligonucleotide dendrimer comprises at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 second single stranded nucleic acid oligonucleotide attachment arms.

13. The nanoparticle of any one of claims to 1 to 10 encapsulated in alginate capsules.

14. A method of treating a tumor in a subject, comprising administering the nanoparticle of any one of claims 1 to 13 into the subject.

15. The method of claim 14, wherein the nanoparticle is administered intravenously or intratumorally.

16. A branched nucleic acid-made dendrimer nanostructure electrostatically complexed with peptides containing amino acids with amine groups, guanidine groups, or positive charges, wherein the branched nucleic acid dendrimer nanostructure provides an electrostatic attachment modality with nucleic acid binding peptides through branching arms that collapse to complex with the nucleic acid binding peptides, wherein at least two or more distinct locations distanced by more than 20 bases apart on the nucleic acid nanostructure are electrostatically contacting and binding the peptides simultaneously.

17. A method for stimulating anti-tumoral effect in a tumor of a subject, comprising injecting into the tumor alginate capsules containing a peptide having the amino acid sequence LLGDFFRKSKEKIGKEFKRIVQRIKDFLRNLVPRTES (SEQ ID NO: 1) complexed with a nucleic acid CpG adjuvant or nucleic acid nanostructure containing an adjuvant.

18. The method of claim 17, wherein the peptide further comprises an antigen sequence.

19. A fusion protein comprising a peptide having the amino acid sequence LLGDFFRKSKEKIGKEFKRIVQRIKDFLRNLVPRTES (SEQ ID NO:1) fused to a peptide antigen sequence, cell penetrating peptide sequence, or ligand-targeted peptide sequence.

20. A method of protecting branched nucleic acid dendrimer nanostructure via electrostatic complexation with peptides.

21. The method of claim 20, wherein the peptides comprise the amino acid sequence (LLGDFFRKSKEKIGKEFKRIVQRIKDFLRNLVPRTES (SEQ ID NO:1).

22. A method of mediating cytosolic delivery of a nucleic acid nanostructure, comprising electrostatically complexing the nucleic acid nanostructures with a peptide having the amino acid sequence LLGDFFRKSKEKIGKEFKRIVQRIKDFLRNLVPRTES (SEQ ID NO:1).

23. A method of enhancing cytosolic delivery of nucleic acid nanostructures, comprising decorating the nucleic acid nanostructures with a branched nucleic acid structure layer.

24. The method of claim 22, further comprising complexing the nucleic acid nanostructures with a peptide having the amino acid sequence (LLGDFFRKSKEKIGKEFKRIVQRIKDFLRNLVPRTES (SEQ ID NO:1).

25. The nanoparticle of claim 1, wherein the peptide (e.g., peptide antigen) comprises cell penetrating peptides and/or antimicrobial peptides at the N- and/or C-terminus for tuning the peptide electrostatic attachment to nucleic acid nanostructures, increased cell uptake and cytosolic delivery, and further may contain at least one peptide cleavage site (e.g., cathepsin cleavage site, furin cleavage site, etc.), and/or at least one immunoproteasome processing site for peptide processing containing the amino acid sequence (LLGDFFRKSKEKIGKEFKRIVQRIKDFLRNLVPRTES (SEQ ID NO:1).

26. The nanoparticle of claim 1, wherein the at least 5, 6, 7, 8, 9, or 10 continuous positively charged amino acids are at the N-terminus of the peptide antigen.

27. The nanoparticle of claim 1, wherein the at least 5, 6, 7, 8, 9, or 10 continuous positively charged amino acids are at the C-terminus of the peptide antigen.

28. The nanoparticle of claim 1, wherein the positively charged amino acids are lysine or arginine amino acids.

29. The nanoparticle of claim 1, wherein the peptide antigen comprises at least contiguous lysine amino acids.

30. The nanoparticle of claim 1, wherein the peptide antigen comprises at least contiguous arginine amino acids.

31. The nanoparticle of claim 1, comprising at least 30, 35, 40, 45, 50, 60, 70, 80, 90, 100 peptides per nm².

32. The nanoparticle of claim 1, wherein each of the plurality of scaffold strands are 300 to 15,000 nucleotides in length.

33. The formulation pertaining to any one of claims 1 to 32, wherein the peptide antigen comprises a viral antigen.

34. The formulation pertaining to any one of claims 1 to 32, wherein the peptide antigen comprises a tumor specific neoantigen and/or tumor associated antigen.

35. The formulation pertaining to any one of claims 1 to 34, wherein the nucleic acid nanostructure comprises one or more first single stranded nucleic acid oligonucleotide attachment arms configured to bind to a first complementary nucleic acid oligonucleotide strands.

36. The formulation pertaining to any one of claims 1 to 35, further comprising a plurality of nucleic acid adjuvant molecules conjugated to first complementary nucleic acid oligonucleotide strands or second complementary nucleic acid oligonucleotide strands.

37. The formulation of claim 36, wherein the plurality of nucleic acid adjuvant molecules are CpG molecules.

38. A nanoparticle of any one of claims 1 and 2, wherein the base nucleic acid nanostructure comprises a single or multiple cavities, wherein the one or more first single stranded oligonucleotide attachment arms containing branched nucleic acid dendrimers are positioned inside and/or outside of the cavity, or within the base nucleic acid nanostructure.

39. A nanoparticle of any one of claims 1 and 2, further comprising one or more targeting ligands conjugated to the first complementary nucleic acid oligonucleotide strands or second complementary nucleic acid oligonucleotide strands.

40. A method for vaccinating a subject, comprising administering to the subject the vaccine device of any one of claims 1 to 39.

41. A non-DNA origami nucleic acid nanostructure formed from a nucleic acid material and optionally therapeutic, targeting, sensing, imaging, detection, and building materials (e.g., phospholipids), comprising a peptide of interest, which is extended via peptide bond (i.e., amide bond) synthesis to contain additional peptide sequence/s that allow attachment of the peptide sequences of interest to the nucleic acid polymers of the nanoparticle by electrostatic or other interactions.

42. The nanoparticle of claim 41, wherein the peptide sequence of interest can be polypeptide or protein.

43. A nucleic acid dendrimer nanostructure that is made by self-assembly of unique predefined sequences at predefined molar ratios in one step (i.e., one pot synthesis) in the presence of only 1× phosphate buffered saline and require no purification procedures after the self-assembly process.

44. A nucleic acid dendrimer nanostructure that is made by self-assembly of unique predefined sequences that do not require oligonucleotide purification such as HPLC or PAGE before self-assembly process and are assembled at predefined molar ratios in one step (i.e., one pot synthesis) in the presence of only 1× phosphate buffered saline and require no purification procedures after the self-assembly process.

45. A nucleic acid nanovaccine delivery platform that can directly incorporate FDA approved oligonucleotide adjuvants (e.g., CpG) by direct attachment to the nucleic acid structure, without the need for modification of oligonucleotide adjuvant, while preserving adjuvant function and potency.

46. The nanoparticle of claim 1, wherein the peptide comprises 1 to 5 contiguous positively charged amino acids.

47. The nanoparticle of claim 1, wherein the peptide comprises 1 to 3 contiguous positively charged amino acids.

48. A nucleic acid structure, comprising a nucleic acid origami nanostructure formed from a plurality of scaffold strands and a plurality of staple strands assembled into a geometry, wherein the nucleic acid nanostructure comprises one or more first single stranded oligonucleotide attachment arms configured to bind to a first complementary oligonucleotide strands, further comprising a branched oligonucleotide dendrimer comprising a plurality of second single stranded oligonucleotide attachment arms and at least one first complementary oligonucleotide strand, wherein the second single stranded oligonucleotide attachment arms are configured to bind to second complementary oligonucleotide strands.


SEQUENCE LISTING (5 sequences)

SEQ ID NO: 1 - protein, 37 aa, synthetic construct
LLGDFFRKSKEKIGKEFKRIVQRIKDFLRNLVPRTES

SEQ ID NO: 2 - protein, 45 aa, synthetic construct
LLGDFFRKSKEKIGKEFKRIVQRIKDFLRNLVPRTESSIINFEKL

SEQ ID NO: 3 - protein, 8 aa, synthetic construct
SIINFEKL

SEQ ID NO: 4 - protein, 17 aa, synthetic construct
KLALRVRRALISLEQLE

SEQ ID NO: 5 - protein, 65 aa, synthetic construct
LLGDFFRKSKEKIGKEFKRIVQRIKDFLRNLVPRTESKLALRVRRALISLEQLESIINFEKLTEW